(19) United States
(12) Patent Application Publication
(10) Pub. No.: US 2025/0333754 A1
(43) Pub. Date: Oct. 30, 2025

(54) HI Genes and Normal A Cytotype Combination

(71) Applicant: SYNGENTA CROP PROTECTION AG, Basel (CH)

(72) Inventors: Timothy Kelliher, Research Triangle Park, NC (US); Brent Delzer, Janesville, WI (US); David Stewart Skibbe, Research Triangle Park, NC (US); Jason Nichols, Research Triangle Park, NC (US)

(73) Assignee: Syngenta Crop Protection AG, Basel (CH)

(21) Appl. No.: 18/282,693

(22) PCT Filed: Mar. 29, 2022
(86) PCT No.: PCT/US22/22271

§ 371 Date: Sep. 18, 2023

Related U.S. Application Data
(60) Provisional application No. 63/169,316, filed on Apr. 1, 2021.

(51) Int. Cl. C12N 15/82; C12N 9/20; C12N 9/22

(52) U.S. Cl. CPC C12N 15/8243; C12N 9/20; C12N 9/22; C12N 2310/20; C12Y 301/01003

(57) ABSTRACT

Provided herein are highly transformable maize plants, referred to as HI-NA plants, and methods of their production and use. A HI-NA plant, as disclosed herein, is homozygous for a loss-of-function mutant allele in the patatin-like phospholipase A2α (MATL) gene and at least heterozygous for one or more QTL and/or gene alleles that are responsible for increased haploid induction and/or transformation frequency in plants. A HI-NA plant, as disclosed herein, may also have a cytotype A background, which may render it highly transformable. Also provided are methods of producing I-NA plants and methods of using a HI-NA plant for editing plant genomic DNA.

### FIELD

This disclosure relates to the field of plant biotechnology. In particular, it relates to plant transformation and plant breeding as well as gene editing, including in plants recalcitrant to accepting foreign transgenes.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 82222_ST25.txt, created on Mar. 25, 2022, and having a size of 231 KB and is filed concurrently with the specification. The sequence listing contained in this ASCII formatted document is part of the specification and is herein incorporated by reference in its entirety.

### BACKGROUND

Plant transformation, that is, the stable integration of foreign DNA (“transgenes”) into a plant genome, has been used for decades to add new and useful traits to crops. While some maize lines are relatively easy to transform (i.e., accepting of transgenic DNA), most lines are not. For example, most elite inbred lines, which are produced by self-pollination over several generations to obtain a pure or nearly pure homozygous genome and which are used as parent lines to create commercially valuable hybrids, often cannot be transformed with foreign DNA. Thus, in order to move a transgenic trait into an inbred line, the transgenic trait must first be transformed into a transformable maize line. That transformed maize line is rarely suitable for use as a parent line in breeding platforms. Therefore, the transformed maize line is crossed into an inbred line to create a progeny plant which will comprise, in a heterozygous manner, the genomes of both the inbred parent and the transformed parent. Then, that progeny plant comprising the transgene must be backcrossed into the inbred line for approximately six or seven generations in order to eliminate, as much as possible, the genome contributed by the transformed parent while retaining the transgenic trait. This trait introgression process generally takes between three to seven years.

Maize is known to have at least five different cytotypes (classified based on mitochondrial genome): normal A (“NA”), normal B (“NB”), cytoplasmic-male-sterile C (“CMS-C” or “C”), cytoplasmic-male-sterile S (“CMS-S” or “S”), and cytoplasmic-male-sterile T (“CMS-T” or “T”). Other cytotypes may still be discovered. Mitochondria and chloroplasts present in these various cytotypes, by way of their genome, may thus have an outsized effect, comparatively speaking, on a plant cell's phenotype. These effects are only now being determined. For example, it was recently discovered that there is a relationship between transformability and cytotype. Maize lines known to be transformable have the NA cytotype, whereas maize lines known to not be transformable (recalcitrant) have the NB cytotype.

Another important tool in plant breeding is haploid induction (“HI”), which is a class of plant phenomena characterized by loss of one parent's set of chromosomes (the chromosomes from the haploid inducer parent) from the embryo at some time during or after fertilization, often during early embryo development. Haploid induction has been observed in numerous plant species, such as sorghum, barley, wheat, maize, Arabidopsis, and many other species. In maize, haploid seed or embryos can be produced by making crosses between a haploid inducer male (i.e., “haploid inducer pollen”) and virtually any ear that one chooses. In the case of maternal HI systems, e.g., matrilineal-based systems, haploids are produced when the haploid inducer pollen DNA is not fully transmitted and/or maintained through the first cell divisions of the embryos. The resulting kernels have haploid embryos that contain only the maternal DNA plus normal (fertilized) triploid endosperm. In the case of paternal HI systems, e.g., CENH3-based or ig1-based systems, haploids are produced after the egg is fertilized by the sperm cell and the maternal chromosomes are lost upon cell division. The resulting kernels have haploid embryos that contain only the paternal DNA plus normal (fertilized) triploid endosperm. Regardless of the HI system used, the resulting phenotype is not fully penetrant, with some ovules containing haploid embryos and others containing diploid embryos, aneuploidy embryos, chimeric embryos, or aborted embryos. After haploid induction, haploid embryos or seeds are typically segregated from diploid and aneuploidy siblings using a phenotypic or genetic marker screen and grown or cultured into haploid plants. These plants are then converted either naturally or via chemical manipulation (e.g., using an anti-microtubule agent such as colchicine) into doubled haploid (“DH”) plants which then produce inbred seed.

The production of DH plants enables plant breeders to obtain inbred lines without multi-generational inbreeding, thus decreasing the time required to produce homozygous plants. DH plants provide an invaluable tool to plant breeders, particularly for generating inbred lines, quantitative trait locus (QTL) mapping, cytoplasmic conversion, trait introgression, and F2 screening for high throughput trait improvement. A great deal of time is spared as homozygous lines are essentially generated in one generation, negating the need for multi-generational single-seed descent (conventional inbreeding). In particular. because DH plants are entirely homozygous, they are very amenable to quantitative genetics studies. The production of haploid seed is critical for the doubled haploid breeding process.

### BRIEF SUMMARY

Plant transformation is challenging, particularly in maize. Few plant lines are naturally transformable; the vast majority are not. Furthermore, haploid inducer lines are challenging to breed with, as they have bizarre reproductive characteristics (e.g., self-deletion of DNA during reproduction). Provided herein are highly transformable maize plants, referred to as “HI-NA plants,” and methods of their production and use. A HI-NA plant, as disclosed herein, is homozygous for a loss-of-function mutant allele in the patatin-like phospholipase A2α gene (which is also referred to in various publications as MATRILINEAL [MATL], NOT LIKE DAD [NLD], and PHOSPHOLIPASEAJ [PLA1] and is indicated by the maize B73 v4 gene ID GRMZM2G471240) and is at least heterozygous for one or more alleles of QTLs and/or genes that are responsible for increased haploid induction in plants. For example, the HI-NA plant can be homozygous for a loss-of-function matl mutant allele and at least heterozygous for a HI allele at the qhir8 QTL. Also, the HI-NA plant has a cytotype Normal A (“NA”) background, which renders it highly transformable. The HI-NA plants provided herein have remarkable haploid induction capability (having a haploid induction rate of at least 12%, at least 15%, or at least 18%) and as well as superior transformability (a transformation rate of at least 2%, at least 5%, at least 8%, at least 10%, at least 12%, or at least 15%.). The HI-NA lines can be produced from plants from a variety of heterotic groups (defined below).

These highly transformable HI-NA plants can be transformed with gene editing machinery to edit the genomic DNA of plant lines of interest to improve plant traits. Such methods are described, for example, in U.S. Pat. Nos. 10,285,348 and 10,519,456, each of which is incorporated by reference herein in its entirety. By providing easily-transformable HI-NA plants that are both strong haploid inducers and highly tranformable, the present disclosure provides useful tools for efficiently and cost effectively editing crop genomes to produce plant lines with desired traits.

In one aspect, provided herein is a maize plant homozygous for a loss-of-function mutation in the patatin-like phospholipase A2α gene (MATL) and at least heterozygous for a HI allele at at least one quantitative trait locus (QTL) associated with increased haploid induction (HI-QTL), wherein the maize plant has a normal A (“NA”) cytotype. In some embodiments, the maize plant is homozygous for the HI allele at the at least one HI-QTL. In some embodiments, the maize plant is at least heterozygous for a TF allele at at least one QTL associated with increased transformation frequency (TF-QTL). In some embodiments, the maize plant is capable of expressing a DNA modification enzyme and optionally at least one guide nucleic acid.

In another aspect, provided herein is a maize plant that is at least heterozygous for a TF allele at at least one quantitative trait locus (QTL) associated with increased transformation frequency (TF-QTL). In some embodiments, the maize plant is homozygous for a TF allele at at least one QTL associated with increased transformation frequency (TF-QTL).

In another aspect, provided herein is a method of producing a transformable haploid inducer maize plant, comprising: a) providing pollen from a first maize plant, wherein the first maize plant is a haploid inducer plant line that is homozygous for a loss-of-function mutation in the patatin-like phospholipase A2α gene (MATL) gene, at least heterozygous for a HI allele at a second locus, and transformation recalcitrant; b) providing a second maize plant, wherein the second maize plant comprises normal A (“NA”) cytoplasm, and, optionally, wherein the second maize plant is at least heterozygous for a TF allele at a quantitative trait locus (QTL) associated with increased transformation frequency (TF-QTL); c) pollinating the second maize plant with the pollen from the first maize plant and obtaining at least one diploid progeny plant therefrom; d) selfing the at least one diploid progeny plant and/or backcrossing the at least one diploid progeny plant to either the first maize plant or the second maize plant for at least one generation; and e) selecting progeny from the crossing of step d, wherein the selected progeny comprises the NA cytotype, is homozygous for the loss-of-function mutation in the MATL gene, is at least heterozygous for the HI allele at the second locus, and, optionally, is at least heterozygous for the TF allele at the TF-QTL.

In another aspect, provided herein is a method of producing a transformable haploid inducer maize plant, comprising: a) providing pollen from a first maize plant, wherein the first maize plant is a haploid inducer plant line that is homozygous for a loss-of-function mutation in the patatin-like phospholipase A2α gene (MATL) gene, at least heterozygous for a HI allele at a second locus, and transformation recalcitrant; b) providing a second maize plant, wherein the second maize plant is at least heterozygous for a TF allele at a quantitative trait locus (QTL) associated with increased transformation frequency (TF-QTL); c) pollinating the second maize plant with the pollen from the first maize plant and obtaining at least one diploid progeny plant therefrom; d) selfing the at least one diploid progeny plant and/or backcrossing the at least one diploid progeny plant to either the first maize plant or the second maize plant for at least one generation; and e) selecting progeny from the crossing of step d, wherein the selected progeny is homozygous for the loss-of-function mutation in the MATL gene, is at least heterozygous for the HI allele at the second locus, and is at least heterozygous for the TF allele at the TF-QTL.

In another aspect, provided herein is a method of producing a transformable haploid inducer maize plant, comprising: a) providing pollen from a first maize plant, wherein the first maize plant is homozygous for a wild-type allele of the patatin-like phospholipase A2α gene (MATL) gene and homozygous for a wild-type allele of the DUF679 domain membrane protein 7 (DMP) gene; b) providing a second maize plant, wherein the second maize plant comprises normal A (“NA”) cytoplasm, and, optionally, wherein the second maize plant is at least heterozygous for a TF allele at a quantitative trait locus (QTL) associated with increased transformation frequency (TF-QTL); c) pollinating the second maize plant with the pollen from the first maize plant and obtaining at least one diploid progeny plant therefrom; d) selfing the at least one diploid progeny plant and/or backcrossing the at least one diploid progeny plant to either the first maize plant or the second maize plant for at least one generation; e) selecting progeny from the crossing of step d, wherein the selected progeny comprises the NA cytotype and, optionally, is at least heterozygous for the TF allele at the TF-QTL; and f) editing at least one progeny plant to cause a loss-of-function mutation in the wild-type MATL gene and/or the DMP gene, thereby obtaining a transformable haploid inducer maize plant.

In another aspect, provided herein is a method of editing plant genomic DNA, comprising: a) providing a target plant, wherein the target plant comprises the plant genomic DNA that is to be edited; b) pollinating the target plant with pollen from a maize plant described herein, wherein the maize plant is capable of expressing a DNA modification enzyme and, optionally, at least one guide nucleic acid; and c) selecting at least one haploid progeny produced by step c, wherein the haploid progeny comprises the genome of the target plant and does not comprise the genome of the maize plant, and the genome of the haploid progeny has been modified by the DNA modification enzyme and optional guide nucleic acid delivered by the maize plant.

In some embodiments, the HI-QTL of any of the above aspects is qhir8 on chromosome 9 (HI-QTL qhir8). In some embodiments, the HI allele at the HI-QTL qhir8 of any of the above aspects comprises a loss-of function mutation in the DUF679 domain membrane protein 7 (DMP) gene. In some embodiments, the TF-QTL of any of the above aspects is qCYTO-A_TF3.1 on chromosome 3 (TF-QTL qCYTO-A_TF3.1).

### BRIEF DESCRIPTION OF THE DRAWINGS

FIG. 1 shows exemplary steps of a process of generating the HI-NA plants according to aspects of this disclosure.

FIG. 2 shows a diagram of the genetic elements in construct 26258.

FIG. 3 shows a diagram of the genetic elements in construct 24288.

### BRIEF DESCRIPTION OF THE SEQUENCES

| SEQ ID NO | Name |
|---|---|
| 1 | SM7246 F primer |
| 2 | SM7246 R primer |
| 3 | SM7246 FAM |
| 4 | SM7246 TET |
| 5 | SM7246 WT |
| 6 | SM7246 variant |
| 7 | SM7252 F primer |
| 8 | SM7252 R primer |
| 9 | SM7252 FAM |
| 10 | SM7252 TET |
| 11 | SM7252 WT |
| 12 | SM7252 variant |
| 13 | Assay 2826 TREM26 F primer |
| 14 | Assay 2826 PM0033 F primer |
| 15 | Assay 2826 TREM26 R primer |
| 16 | Assay 2826 PM0033 R primer |
| 17 | Assay 2826 FAM |
| 18 | Assay 2826 TET |
| 19 | Assay 2827 TREM27 F primer |
| 20 | Assay 2827 TREM27 R primer |
| 21 | Assay 2827 FAM |
| 22 | SM4849 F primer |
| 23 | SM4849 R primer |
| 24 | SM4849 FAM |
| 25 | SM4849 TET |
| 26 | SM4849 WT |
| 27 | SM4849 variant |
| 28 | SM8047 F primer |
| 29 | SM8047 R primer |
| 30 | SM8047 FAM |
| 31 | SM8047 TET |
| 32 | SM8047 WT |
| 33 | SM8047 variant |
| 34 | SM8133 F primer |
| 35 | SM8133 R primer |
| 36 | SM8133 FAM |
| 37 | SM8133 TET |
| 38 | SM8133 WT |
| 39 | SM8133 variant |
| 40 | SM8029 F primer |
| 41 | SM8029 R primer |
| 42 | SM8029 FAM |
| 43 | SM8029 TET |
| 44 | SM8029 WT |
| 45 | SM8029 variant |
| 46 | SM4257 F primer |
| 47 | SM4257 R primer |
| 48 | SM4257 FAM |
| 49 | SM4257 TET |
| 50 | SM4257 WT |
| 51 | SM4257 variant |
| 52 | SM0956BQ F primer |
| 53 | SM0956BQ R primer |
| 54 | SM0956BQ FAM |
| 55 | SM0956BQ TET |
| 56 | SM0956BQ WT |
| 57 | SM0956BQ variant |
| 58 | SM0954BQ F primer |
| 59 | SM0954BQ R primer |
| 60 | SM0954BQ FAM |
| 61 | SM0954BQ TET |
| 62 | SM0954BQ WT |
| 63 | SM0954BQ variant |
| 64 | SM0954HQ F primer |
| 65 | SM0954HQ R primer |
| 66 | SM0954HQ FAM |
| 67 | SM0954HQ TET |
| 68 | SM0954HQ WT |
| 69 | SM0954HQ variant |
| 70 | SM6568 F primer |
| 71 | SM6568 R primer |
| 72 | SM6568 FAM |
| 73 | SM6568 TET |
| 74 | SM6568 WT |
| 75 | SM6568 variant |
| 76 | SM0953BQ F primer |
| 77 | SM0953BQ R primer |
| 78 | SM0953BQ FAM |
| 79 | SM0953BQ TET |
| 80 | SM0953BQ WT |
| 81 | SM0953BQ variant |
| 82 | SM6604 F primer |
| 83 | SM6604 R primer |
| 84 | SM6604 FAM |
| 85 | SM6604 TET |
| 86 | SM6604 WT |
| 87 | SM6604 variant |
| 88 | SM8040 F primer |
| 89 | SM8040 R primer |
| 90 | SM8040 FAM |
| 91 | SM8040 TET |
| 92 | SM8040 WT |
| 93 | SM8040 variant |
| 94 | SM8091 F primer |
| 95 | SM8091 R primer |
| 96 | SM8091 FAM |
| 97 | SM8091 TET |
| 98 | SM8091 WT |
| 99 | SM8091 variant |
| 100 | SM2918 F primer |
| 101 | SM2918 R primer |
| 102 | SM2918 FAM |
| 103 | SM2918 TET |
| 104 | SM2918 normal |
| 105 | SM2918 CMS |
| 106 | SM4813 F primer |
| 107 | SM4813 R primer |
| 108 | SM4813 FAM |
| 109 | SM4813 TET |
| 110 | SM4813 normal |
| 111 | SM4813 CMS |
| 112 | SM2914 F primer |
| 113 | SM2914 R primer |
| 114 | SM2914 FAM |
| 115 | SM2914 TET |
| 116 | SM2914 normal A |
| 117 | SM2914 normal B |
| 118 | SM4812 F primer |
| 119 | SM4812 R primer |
| 120 | SM4812 FAM |
| 121 | SM4812 TET |
| 122 | SM4812 normal A |
| 123 | SM4812 normal B |
| 124 | MATL WT cDNA |
| 125 | Example matl variant cDNA |
| 126 | DMP |
| 127 | Waxy1 probe |
| 128 | Waxy1 gRNA |
| 129 | SM3158 F primer |
| 130 | SM3158 R primer |
| 131 | SM3158 FAM |
| 132 | SM3158 TET |
| 133 | SM3158 RWKS |
| 134 | SM3158 SYN-INBC34 |
| 135 | SM4787 F primer |
| 136 | SM4787 R primer |
| 137 | SM4787 FAM |
| 138 | SM4787 TET |
| 139 | SM4787 RWKS |
| 140 | SM4787 SYN-INBC34 |
| 141 | SM3814 F primer |
| 142 | SM3814 R primer |
| 143 | SM3814 FAM |
| 144 | SM3814 TET |
| 145 | SM3814 RWKS |
| 146 | SM3814 SYN-INBC34 |
| 147 | SM3362 F primer |
| 148 | SM3362 R primer |
| 149 | SM3362 FAM |
| 150 | SM3362 TET |
| 151 | SM3362 RWKS |
| 152 | SM3362 SYN-INBC34 |
| 153 | SM0634AQ F primer |
| 154 | SM0634AQ R primer |
| 155 | SM0634AQ FAM |
| 156 | SM0634AQ TET |
| 157 | SM0634AQ RWKS |
| 158 | SM0634AQ SYN-INBC34 |
| 159 | SM4586 F primer |
| 160 | SM4586 R primer |
| 161 | SM4586 FAM |
| 162 | SM4586 TET |
| 163 | SM4586 RWKS |
| 164 | SM4586 SYN-INBC34 |
| 165 | gRNA 1 (DMP) |
| 166 | gRNA 2 (DMP) |
| 167 | gRNA 3 (DMP) |
| 168 | gRNA 4 (MATL) |
| 169 | gRNA 5 (MATL) |
| 170 | gRNA 6 (MATL) |
| 171 | vector 26258 |
| 172 | Opaque2 gRNA target sequence |
| 173 | Waxy1 gRNA target sequence |
| 174 | Yellow Endosperm1 gRNA target sequence |
| 175 | E3 ubiquitin ligase2 gRNA target sequence |
| 176 | putative ubiquitin-protein ligase gRNA target sequence |
| 177 | vector 24288 |
| 178 | cSbWUS-01 |
| 179 | cBnBBM1-02 |
| 180 | vector 25072 |
| 181 | cBdWOX5/7-v1 |
| 182 | maize Ubi1 promoter |
| 183 | FE12949 forward primer |
| 184 | FE12950 reverse primer |
| 185 | FE12951 probe |
| 186 | FE12703 forward primer |
| 187 | FE12704 reverse primer |
| 188 | FE12705 probe |
| 189 | FE12958 forward primer |
| 190 | FE12959 reverse primer |
| 191 | FE12960 probe |
| 192 | FE12952 forward primer |
| 193 | FE12953 reverse primer |
| 194 | FE12954 probe |
| 195 | FE12952 forward primer |
| 196 | FE12956 reverse primer |
| 195 | FE12957 probe |

### DETAILED DESCRIPTION

### I. Terminology

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject.

As used in herein, the singular forms “a” “an” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field, for example ±20%, ±10%, or ±5%, are within the intended meaning of the recited value.

As used herein, the term “comprising” or “comprise” is open-ended. When used in connection with a subject nucleic acid (or amino acid sequence), it refers to a nucleic acid sequence (or an amino acid sequence) that includes the subject sequence as a part or as its entire sequence.

The term “plurality” refers to more than one entity. Thus, a “plurality of individuals” refers to at least two individuals. In some embodiments, the term plurality refers to more than half of the whole. For example, in some embodiments a “plurality of a population” refers to more than half the members of that population.

A “plant” is any plant at any stage of development, particularly a seed plant. In particular, in the context of this disclosure, a plant refers to a maize plant.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any group of plant cells organized into structural and/or functional units. The use of this term in conjunction with, or in the absence of, any specific type of plant tissue as listed above or otherwise embraced by this definition is not intended to be exclusive of any other type of plant tissue.

The term “plant part” indicates a part of a plant, including single cells and cell tissues such as plant cells that are intact in plants, cell clumps and tissue cultures from which plants can be regenerated. Examples of plant parts include, but are not limited to, single cells and tissues from pollen, ovules, zygotes, leaves, embryos, roots, root tips, anthers, flowers, flower parts, fruits, stems, shoots, cuttings, and seeds; as well as pollen, ovules, egg cells, zygotes, leaves, embryos, roots, root tips, anthers, flowers, flower parts, fruits, stems, shoots, cuttings, scions, rootstocks, seeds, protoplasts, calli, and the like.

The terms “variety” or “cultivar” mean a group of similar plants that by structural or genetic features and/or performance can be distinguished from other varieties within the same species.

The term “population” means a genetically heterogeneous collection of plants sharing a common genetic derivation.

The term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

The term “offspring” plant refers to any plant resulting as progeny from a vegetative or sexual reproduction from one or more parent plants or descendants thereof. For instance, an offspring plant may be obtained by cloning or selfing of a parent plant or by crossing two parent plants and includes selfings as well as the F1 or F2 or still further generations. An F1 is a first-generation offspring produced from parents at least one of which is used for the first time as donor of a trait, while offsprings of second generation (F2) or subsequent generations (F3, F4, etc.) are specimens produced from selfings of F1's, F2's etc. An F1 may thus be a hybrid resulting from a cross between two true breeding parents (true-breeding is homozygous for a trait), while an F2 may be an offspring resulting from self-pollination of said F1 hybrids.

The phrases “sexually crossed” and “sexual reproduction” in the context of the present disclosure refer to the fusion of gametes to produce progeny (e.g., by fertilization, such as to produce seed by pollination in plants). In some embodiments, a “sexual cross” or “cross-fertilization”is fertilization of one individual by another (e.g., cross-pollination in plants). In some embodiments the term “selfing” refers to the production of seed by self-fertilization or self-pollination; i.e., pollen and ovule are from the same plant.

“Selective breeding” is understood within the scope of the present disclosure to refer to a program of breeding that uses plants that possess or display desirable traits as parents.

The terms “hybrid”, “hybrid plant”, and “hybrid progeny” in the context of plant breeding refer to a plant that is the offspring of genetically dissimilar parents produced by crossing plants of different lines or breeds or species, including but not limited to the cross between two inbred lines (e.g., a genetically heterozygous or mostly heterozygous individual). The phrase “single cross FI hybrid” refers to an FI hybrid produced from a cross between two inbred lines.

The phrase “inbred line” refers to a genetically homozygous or nearly homozygous population. An inbred line, for example, can be derived through several cycles of brother/sister breedings or of selfing. In some embodiments, inbred lines breed true for one or more phenotypic traits of interest. An “inbred”, “inbred individual”, or “inbred progeny” is an individual sampled from an inbred line. The term “inbred” means a substantially homozygous individual or line. An inbred line may also be referred to as a “parent line” when used in a breeding program.

The term “backcrossing” is understood within the scope of the present disclosure to refer to a process in which a hybrid progeny is repeatedly crossed back to one of the parents.

The terms “introgression”, “introgressed”, and “introgressing” refer to both a natural and artificial process whereby genomic regions of one species, variety or cultivar are moved into the genome of another species, variety or cultivar, by crossing those species. The process may optionally be completed by backcrossing to the recurrent parent.

A plant referred to herein as “haploid” has a reduced number of chromosomes (n) in the haploid plant, and its chromosome set is equal to that of the gamete. In a haploid organism, only half of the normal number of chromosomes are present. Thus haploids of diploid (2n) organisms (e.g., maize) exhibit monoploidy (In); haploids of tetraploid (4n) organisms (e.g., ryegrasses) exhibit diploidy (2n); haploids of hexaploid (6n) organisms (e.g., wheat) exhibit triploidy (3n); etx. As used herein, a plant referred to as “doubled haploid” is developed by doubling the haploid set of chromosomes. A plant or seed that is obtained from a doubled haploid plant that is selfed to any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes; that is, a plant will be considered doubled haploid if it contains viable gametes, even if it is chimeric in vegetative tissues.

“Recombination” is the exchange of DNA strands to produce new nucleotide sequence arrangements. The term may refer to the process of homologous recombination that occurs in double-strand DNA break repair, where a polynucleotide is used as a template to repair an homologous polynucleotide. The term may also refer to exchange of information between two homologous chromosomes during meiosis. The frequency of double recombination is the product of the frequencies of the single recombinants. For instance, a recombinant in a 10 cM area can be found with a frequency of 10%, and double recombinants are found with a frequency of 10%×10%=1% (1 centimorgan is defined as 1% recombinant progeny in a testcross).

“Tester plant” is understood within the scope of the present disclosure to refer to a plant used to characterize genetically a trait in a plant to be tested. Typically, the plant to be tested is crossed with a “tester” plant and the segregation ratio of the trait in the progeny of the cross is scored.

The term “tester” refers to a line or individual with a standard genotype, known characteristics, and established performance. A “tester parent” is an individual from a tester line that is used as a parent in a sexual cross. Typically, the tester parent is unrelated to and genetically different from the individual to which it is crossed. A tester is typically used to generate FI progeny when crossed to individuals or inbred lines for phenotypic evaluation.

The terms “heterotic group” and “heterotic pool” are used interchangeably and refer to a group of genotypes or inbred lines that demonstrate similar heterotic response when crossed with genotypes or inbred lines from other genetically distinct germplasm groups. There is a closer degree of genetic relationship of lines contained within a heterotic group versus the more distant degree of genetic relationship of lines compared between heterotic groups. In general, the hybrid of two inbred lines crossed together within the same heterotic group shows much less heterosis than the hybrid of an inbred line from one heterotic group crossed to an inbred line from a different heterotic group. A particular heterotic group can include multiple lines having diverse genetics. Exemplary heterotic groups and proprietary germplasm lines within each individual heterotic group are described in Table 7. In the present disclosure, the totality of genotypes of an entire heterotic group may also be referred to as the germplasm of the heterotic group. Broadly, the primary designations for heterotic pools are: Stiff Stalk (“SS,” also called Iowa Stiff Stalk Synthetic, or “BSSS”), Non-Stiff Stalk (“NSS”), Tropical, and Non-Stiff Stalk Iodent (“IDT”). See J. Hweerwaarden, et al., Historical genomics of North American maize, PROC. NAT'L ACAD. SCI. U.S.A. 109(31): 12420-25 (2012). These are not exclusive, however, and other designations are known, e.g., Lancaster Sure Crop (“LSC”). See, e.g., C. Livini, et al., Genetic diversity of maize inbred lines with and among heterotic groups revealed by RFLPs, THEOR. APPL. GENET. 84: 17-25 (1992).

The term “heterosis” refers to hybrid vigor, i.e., the improved or increased function of any biological quality (e.g., size, growth rate, fertility, yield, etc.) in a hybrid offspring relative to its parents. For example, the offspring of a cross between inbred plant lines from different heterotic groups is likely to display more heterosis than its parent lines, as described above. The first-generation offspring of such a cross generally show, in greater measure, the desired characteristics of both parents. This heterosis may decrease in subsequent generations if the first-generation hybrids are mated together.

The term “seed set” refers to a measure of the portion of a maize ear that produces embryos (i.e., kernels or seeds). Seed set may be expressed qualitatively (e.g., low, good, or high) or quantitatively. In a quantitative measurement, the measurement may be given as either a percentage or a number of seeds per ear. The term generally refers to the percentage or number of normal kernels (i.e. non-aborted, endosperm-viable kernels). For normal maize lines (i.e. not haploid inducer lines), a seed set above 80% (or above 300 kernels per ear) is considered a good seed set. For haploid inducer lines, seed set tends to be lower, so a seed set above 50% (e.g., above 60%, above 70%, or above 80%) or above 180 kernels per ear (e.g., above 200, above 220, above 260, or above 280) is generally considered a high seed set.

The terms “nucleic acid” and “polynucleotide” are used interchangeably and as used herein refer to both sense and anti-sense strands of RNA, cDNA, genomic DNA, mitochondrial DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA and/or RNA. In addition, a polynucleotide disclosed herein, e.g., a circular DNA template, a nucleic acid concatemer disclosed herein, may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. Nucleotide sequences are “complementary” when they specifically hybridize in solution (e.g., according to Watson-Crick base pairing rules). The term also includes codon-optimized nucleic acids that encode the same polypeptide sequence. It is also understood that nucleic acids can be unpurified, purified, or attached, for example, to a synthetic material such as a bead or column matrix.

The term “corresponding to” in the context of nucleic acid sequences means that when the nucleic acid sequences of certain sequences are aligned with each other, the nucleic acids that “correspond to” certain enumerated positions in the present invention are those that align with these positions in a reference sequence, but that are not necessarily in these exact numerical positions relative to a particular nucleic acid sequence of the invention. Optimal alignment of sequences for comparison can be conducted by computerized implementations of known algorithms. or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif, United States of America. See also Smith & Waterman, 1981; Needleman & Wunsch, 1970; Pearson & Lipman, 1988; Ausubel et al., 1988; and Sambrook & Russell, 2001.

The term “gene” refers to a hereditary unit including a sequence of DNA that occupies a specific location on a chromosome and that contains the genetic instruction for a particular characteristic or train in an organism.

The term “quantitative trait locus” or “QTL” refers to a region of DNA that is associated with a particular phenotypic trait, i.e. a phenotype that can be measured numerically and varies in degree, and which can be attributed to polygenic effects, i.e., the product of two or more genes, and their environment. Typically, QTLs underlie continuous traits (those traits which vary continuously, e.g. haploid induction rate) as opposed to qualitative (i.e. discrete) traits.

The term “allele(s)” means any of one or more alternative forms of a gene, all of which alleles relate to at least one trait or characteristic. In a diploid cell, the two alleles of a given gene occupy corresponding loci on a pair of homologous chromosomes. In some instances (e.g., for QTLs) it is more accurate to refer to “haplotype” (i.e., an allele of a chromosomal segment) instead of “allele”, however, in those instances, the term “allele” should be understood to comprise the term “haplotype”. If two individuals (e.g., two plants) possess the same allele at a particular locus, the alleles are termed “identical by descent” if the alleles were inherited from one common ancestor (i.e., the alleles are copies of the same parental allele). The alternative is that the alleles are “identical by state” (i.e., the alleles appear to be the same but are derived from two different copies of the allele). Identity by descent information is useful for linkage studies; both identity by descent and identity by state information can be used in association studies, although identity by descent information can be particularly useful.

The term “haplotype” can refer to the set of alleles an individual inherited from one parent. A diploid individual thus has two haplotypes. The term “haplotype” can be used in a more limited sense to refer to physically linked and/or unlinked genetic markers (e.g., sequence polymorphisms) associated with a phenotypic trait. The phrase “haplotype block” (sometimes also referred to in the literature simply as a haplotype) refers to a group of two or more genetic markers that are physically linked on a single chromosome (or a portion thereof). Typically, each block has a few common haplotypes, and a subset of the genetic markers (i.e., a “haplotype tag”) can be chosen that uniquely identifies each of these haplotypes.

The term “genotype” and variants thereof refers to the genetic composition of an organism, including, for example, whether a diploid organism is heterozygous (i.e., has two different alleles for a given gene or QTL) or homozygous (i.e., has the same allele for a given gene or QTL) for one or more genes or loci (e.g., a SNP, a haplotype, a gene mutation, an insertion, or a deletion). As used herein, the term “at least heterozygous” for a particular allele indicates that at least one copy of the allele is present. For example, a maize plant that is at least heterozygous for a HI allele of a gene has either one or two copies (i.e., is either heterozygous or homozygous) of the HI allele.

“Phenotype” is understood within the scope of the present disclosure to refer to a distinguishable characteristic(s) of a genetically controlled trait. The phrase “phenotypic trait” refers to the appearance or other detectable characteristic of an individual, resulting from the interaction of its genome with the environment.

The phrase “qualitative trait” refers to a phenotypic trait that is controlled by one or a few genes that exhibit major phenotypic effects that can be described as a category having two or more character values. Because of this, qualitative traits are typically simply inherited. Examples in plants include, but are not limited to, flower color, cob color, and disease resistance such as, for example, Northern corn leaf blight resistance.

The term “polymorphism” and variants thereof refers to the occurrence of two or more genetically determined alternative sequences or alleles in a population. A “polymorphic site” refers to the locus at which divergence occurs. Preferred polymorphic sites have at least two alleles, each occurring at a particular frequency in a population. A polymorphic locus may be as small as one base pair. One of the alleles of a polymorphism is arbitrarily designated as the reference allele, and other alleles are designated as alternative alleles, “variant alleles,” or “variances.” The allele occurring most frequently in a selected population can sometimes be referred to as the “wild-type” allele. Diploid organisms may be homozygous or heterozygous for the variant alleles. The variant allele may or may not produce an observable physical or biochemical characteristic (phenotype) in an individual carrying the variant allele. For example, a variant allele may alter the enzymatic activity of a protein encoded by a gene of interest or in the alternative the variant allele may have no effect on the enzymatic activity of an encoded protein.

As used herein the terms “marker,” “polymorphic marker,” or “genetic marker” refer to a gene or DNA sequence with a known chromosomal locus that indicates the presence or absence of an allele. A marker may be within or linked to the gene it is used to genotype. A marker can be derived from genomic nucleotide sequences or from encoded products thereof (e.g., an mRNA transcript, a noncoding RNA transcript, or a protein). The term also refers to nucleotide sequences complementary to or flanking the marker sequences, such as nucleotide sequences used as probes and/or primers capable of amplifying the marker sequence. The term can also refer to an absence of nucleotide sequences complementary to or flanking a polymorphism. Markers may include, but are not limited to, single nucleotide polymorphisms (SNPs), single nucleotide variants (SNVs), small insertions or deletions (indels), restriction fragment length polymorphisms (RFLPs), variable number of tandem repeats (VNTR's), hypervariable regions, minisatellites, dinucleotide repeats, trinucleotide repeats, tetranucleotide repeats, simple sequence repeats, and insertion elements such as transposons.

The term “loss-of-function mutation” is a change in the DNA sequence of a gene (i.e., a “mutation”) that results in the mutated gene product lacking the molecular function of the wild-type gene. There are four main genetic variations that can lead to loss-of-function mutations: 1) a mutation resulting in a premature stop codon producing a truncated protein sequence; 2) a mutation occurring at a canonical splice site that affects splicing (resulting in inclusion of an intron or exclusion of an exon in the mRNA transcript); 3) an insertion or deletion variant with non-integral multiples of three located in the gene coding region, causing frameshifts by disrupting the full-length transcript; and 4) mutations that result in the loss of an initiation codon (transcription start codon, e.g. ATG), which prevent gene transcription if there is no alternative start codon near the mutation. In addition, mutations in the promoter or untranslated regions (UTRs) of a gene can reduce or eliminate gene expression, leading to a loss-of-function.

The term “marker-based selection” is understood within the scope of the present disclosure to refer to the use of genetic markers to detect one or more nucleic acids from the plant, where the nucleic acid is associated with a desired trait to identify plants that carry genes for desirable (or undesirable) traits, so that those plants can be used (or avoided) for any purpose, e.g., in a transformation program or in a selective breeding program. As used herein, a marker indicative of Normal A cytoplasm would discriminate between non-CMS plants having Normal B cytoplasm and those not having the Normal B cytoplasm, i.e., having the Normal A cytoplasm. A marker may be a mutation within a locus of a genome (e.g., a single nucleotide polymorphism (“SNP”) or a mutation within one allele.

The terms “marker probe” and “probe,” as used herein, refer to a nucleotide sequence or nucleic acid molecule that can be used to detect the presence or absence of a sequence (e.g., a marker disclosed herein) within a larger sequence. In some embodiments, a nucleic acid probe is complementary to all or a portion of the marker or marker locus and can detect the presence or absence of the marker through, e.g., nucleic acid hybridization. The length of the marker probe may vary. In some embodiments, a marker probe has a length in a range of 8-200 nucleotides, e.g, between 10 and 100 nucleotides, or between 15 and 60 nucleotides. In some embodiments, about 8, 10, 15, 20, 30, 40, 50, 60, 70, 80, 90, 100 or more contiguous nucleotides of the probe are complementary to the marker and can be used for nucleic acid hybridization.

As used herein, the term “primer” refers to an oligonucleotide that is capable of annealing to a nucleic acid target (in some embodiments, annealing specifically to a nucleic acid target) allowing a DNA polymerase and/or reverse transcriptase to attach thereto, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, one or more pluralities of primers are employed to anlplify plant nucleic acids (e.g., using the polymerase chain reaction; PCR).

The term “associated with” as used herein refers to a recognizable and/or assayable relationship between two entities. For example, the phrase “associated with haploid induction (HI)” refers to a trait, locus, gene, allele, marker, phenotype, etc., or the expression product thereof, the presence or absence of which can influence or indicate an extent and/or degree to which a plant or its progeny exhibits HI. As such, a marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence (or absence) of the marker is an indicator of whether the allele is present (or absent) in a plant, germplasm, or population comprising the marker. For example, “a marker associated with HI” refers to a marker whose presence or absence can be used to predict whether and/or to what extent a plant will display HI.

As used herein, the term “plant material” refers to seeds, embryos, or other regenerative tissue coming from a single ear of maize or a set of ears, or plants grown therefrom.

As used herein, the term “plant line” refers to a single plant material or a genetically identical set of materials.

The term “germplasm” refers to the totality of the genotypes of a population or other group of individuals (e.g., a species or plant line). The phrase “adapted germplasm” refers to plant materials of proven genetic superiority; e.g., for a given environment or geo-graphical area, while the phrases “non-adapted germplasm”, “raw germplasm”, and “exotic germplasm” refer to plant materials of unknown or unproven genetic value; e.g., for a given environment or geographical area; as such, the phrase “non-adapted germplasm” refers in some embodiments to plant materials that are not part of an established breeding population and that do not have a known relationship to a member of the established breeding population.

The term “cytotype” refers to the classification of the cytoplasm, including the genetic contribution of the mitochondria and chloroplasts, associated with a plant line. Presently known cytotypes include normal A (“NA”) and normal B (“NB”) cytoplasm, but also include the cytoplasmic male sterile cytotypes: cytoplasmic-male-sterile C (“C” or “CMS-C”) cytoplasm, cytoplasmic-male-sterile S (“S” or “CMS-S”) cytoplasm, and cytoplasmic-male-sterile T (“T” or “CMS-T”) cytoplasm. The terms cytotype and cytoplasm are used interchangeably.

“Transformable,” “transformability,” and the like, refers to a plant, a line of plants, or a plant cell (such as callus tissue or a protoplast) that is more readily accepting of foreign DNA and can stably integrate the foreign DNA into its genome.

“Transformation frequency,” “TF”, “Transformation efficiency,” and “transformation rate” mean a measure of the number of successfully transformed plants divided by the number of total plants (e.g., embryos) that were attempted to be transformed. This measure may be expressed quantitatively, e.g., as a percentage, a raw number, or qualitatively, e.g., “low” or “high.”

The term “TF allele” refers to an allele of a gene or locus the presence of which in a plant (e.g., a maize plant) is associated with increased TF as compared to the alternative alleles for the same gene or locus. In some cases, a TF allele is an allele of a gene, QTL, or locus in a QTL.

The term “TF-QTL” refers to a QTL that is associated with increased transformation frequency (TF). The presence of a TF allele at a TF-QTL results in increased TF as compared to when a non-TF allele is present at the TF-QTL.

As used herein, “recalcitrant” refers to a plant line that is not transformable or essentially not transformable. In other words, its transformation efficiency is 0% or essentially 0%. The term recalcitrant is synonymous with “nontransformable,” and these terms are used interchangeably.

The term “haploid induction rate” or “HIR,” refers to the number of surviving haploid kernels divided by the total number of kernels after an ear is pollinated with haploid inducer pollen.

The term “HI allele” refers to an allele of a gene or locus the presence of which in a plant (e.g., a maize plant) is associated with increased HI as compared to the alternative alleles for the same gene or locus. In some cases, a HI allele is an allele of a gene, a QTL, or a locus in a QTL.

The term “HI-QTL” refers to a QTL that is associated with haploid induction (HI). The presence of a HI allele at a HI-QTL results in increased haploid induction rate (HIR) as compared to when a non-HI allele is present at the HI-QTL. An exemplary HI-QTL is qhir8, located on chromosome 9 between position 3,444,422 and position 11,360,090 in the B73v5 reference genome.

### II. HI-NA Maize Plants

In one aspect, provided herein are maize plants that possess at least two characteristics: 1) the ability to efficiently induce haploid induction; and 2) a high level of transformability. In some embodiments, the maize plants are homozygous for a loss-of-function mutation in a patatin-like phospholipase A2α (MATL) gene and at least heterozygous for a HI allele at at least one HI-QTL. In some embodiments, the HI-QTL can be qhir8 (located on chromosome 9 between position 3,444,422 and position 11,360,090 in the B73v5 reference genome). The maize plants provided herein also have a normal A (“NA”) cytotype, which contributes, in some embodiments, to increased transformability. In some embodiments, the maize plants are at least heterozygous for a TF allele at at least one gene or QTL associated with increased transformability. In some embodiments, the maize plants also exhibit high pollen load and/or tassel weight.

### A. Haploid Induction

Commonly, during haploid induction breeding, both parent lines used in the induction cross are diploid, so their gametes (i.e. egg cells and sperm cells) are haploid. Haploid induction is frequently a medium to low penetrance trait of the inducer line, so the resulting progeny, depending on the species or situation, may be either diploid (if no genome elimination takes place) or haploid (if genome elimination occurs). Therefore, as used herein, “haploids” possess half the number of chromosomes of either parent; thus haploids of diploid organisms (e.g., maize) exhibit monoploidy; haploids of tetraploid organisms (e.g., ryegrasses) exhibit diploidy; haploids of hexaploid organisms (e.g., wheat) exhibit triploidy, and so on.

In some embodiments, haploid induction is achieved by crossing the haploid inducer male line to another line, which results in induction of loss of the set of chromosomes from the haploid inducer line and production of haploid embryos (i.e., efficient haploid induction). Haploid induction efficiency can be represented as haploid induction rate (“HIR”), which is the percentage of total progeny embryos that are haploid from a cross between a haploid inducer line and another line. Exemplary methods for determining HIR are described in Section II.B and also in the Examples of this disclosure. As described herein, variant HI alleles at several genomic loci can promote efficient haploid induction (e.g., HIR of at least 5%, at least 10%, at least 12%, or at least 15%). In some embodiments, the HI allele is an allele of the patatin-like phospholipase A2α gene (PLPA2α, maize B73 gene ID GRMZM2G471240 on chromosome 1 [this gene ID is from the B73_v4 genome] also known as Zm00001d029412 [B73_v5], also known as MATRILINEAL [MATL], NOT LIKE DAD1 [NLD1], and PHOSPHOLIPASE A1 [PLA1]). Also as described herein, HI alleles at various HI-QTLs can also promote haploid induction. In some embodiments, the HI allele may be at the qhir8 HI-QTL on chromosome 9.

In some embodiments, the maize plants disclosed herein comprise a HI allele at the MATL gene. In some embodiments, the HI allele is a loss-of-function mutation in MATL (generally referred to as matl). In some embodiments, the variant allele comprises a four basepair insertion frameshift mutation in the MATL coding sequence. In some embodiments, the four basepair insertion corresponds to the four nucleotides at positions 1146-1149 of SEQ ID NO:125. In some embodiments, the variant allele comprises a different mutation (i.e., other than the four basepair insertion mutation) or different mutations resulting in a loss-of-function in the protein product encoded by MATL. Any assay that is able to identify a loss-of-function mutation in MATL may be used to identify the plants described herein. In some embodiments, the assay may comprise one of the genotyping methods described in Section II.E below. In some embodiments, the assay for identifying a loss-of-function mutation in MATL may be developed based on the wild-type cDNA sequence of the gene (SEQ ID NO:124).

In some embodiments, the assay to identify a loss-of-function mutation in MATL comprises genotyping an individual at one or more of the markers SM7246, SM7252, Assay 2826, and Assay 2827. In some embodiments, the genotypes at these markers may be detected using a TaqMan© real-time PCR assay (e.g., according to the methods detailed in Section II.E or in Example 1 herein). Table 1 lists expected genotypes and sequence contexts at each of these markers, according to some embodiments, along with example primers and probes which can be used in TaqMan real-time PCR genotyping assays. The TaqMan assays described in Table 1 for markers SM7246 and SM7252 each comprise two probes with different fluorophores that can distinguish between the listed genotypes. The TaqMan assays described in Table 1 for the Assay 2826 and Assay 2827 each involve amplification and fluorescent probe-based detection of a portion of the MATL genomic locus and a control for comparison. In some embodiments, the MATL-specific probe in Assay 2826 detects the wild-type MATL sequence (i.e., the mutant sequence is not detected). In some embodiments, the matl-specific probe in Assay 2827 detects the loss-of-function mutant matl sequence with a 4 bp insertion (i.e., the wild-type sequence is not detected).

TABLE 1
Exemplary markers used to genotype a loss-of-function mutation in MATL.

| Marker | SM7246 | SM7252 | Assay 2826 | Assay 2827 |
|---|---|---|---|---|
| Location (B73v5 | Chr 1 | Chr 1 | Chr 1 | Chr 1 |
| reference genome) | 69430544 | 69430644 | 69430644 | 69430644 |
| Wild-type MATL | C/C | D/D | D/D | D/D (not detected) |
| genotype and | SEQ ID NO: 5 | SEQ ID NO: 11 | | |
| sequence context | | | | |
| Mutant matl | G/G | I/I | I/I (not detected) | I/I |
| genotype and | SEQ ID NO: 6 | SEQ ID NO: 12 | | |
| sequence context | | | | |
| F primer | SEQ ID NO: 1 | SEQ ID NO: 7 | SEQ ID NO: 13 (MATL) | SEQ ID NO: 19 (matl) |
| | | | SEQ ID NO: 14 (control) | SEQ ID NO: 14 (control) |
| R primer | SEQ ID NO: 2 | SEQ ID NO: 8 | SEQ ID NO: 15 (MATL) | SEQ ID NO: 20 (matl) |
| | | | SEQ ID NO: 16 (control) | SEQ ID NO: 16 (control) |
| Probes | SEQ ID NOS: | SEQ ID NOS: | SEQ ID NO: 17 (MATL) | SEQ ID NO: 21 (matl) |
| | 3-4 | 9-10 | SEQ ID NO: 18 (control) | SEQ ID NO: 18 (control) |

C/C: homozygous for cytosine at marker; G/G: homozygous for guanine at marker; I/I: homozygous for 4 bp insertion mutant allele at marker; D/D: homozygous for WT allele without 4 bp insertion at marker.

In some embodiments, the assay for identifying a loss-of-function mutation in MATL may be a phenotypic assay. For example, levels of the protein encoded by the mutated MATL sequence may be detected by any of a variety of methods known to those of skill in the art (e.g., Western blot, immunofluorescence, mass spectrometry, etc.). In some embodiments, functional assays may be used to determine if the protein encoded by mutated MATL sequence is able to perform its usual function. For example, plants comprising putative MATL mutations may be crossed to tester plants to assess traits relevant to normal function of the protein encoded by MATL (e.g., seed set or haploid induction rate, as detailed in the Examples below).

In some embodiments, the maize plants disclosed herein comprise a HI allele at at least one quantitative trait locus (QTL) allele associated with increased haploid induction (HI-QTL). In some embodiments, the maize plants are at least heterozygous (e.g., heterozygous or homozygous) for a HI allele at at least one HI-QTL. In some embodiments, the maize plants are homozygous for a HI allele at at least one HI-QTL. In some embodiments, maize plants that are homozygous for a HI allele at a HI-QTL display more efficient haploid induction relative to maize plants that are heterozygous for the HI allele at the HI-QTL. In some embodiments, the maize plants comprise a HI allele at the qhir8 HI-QTL on chromosome 9. Any assay that is able to identify or genotype a QTL may be used to identify the plants comprising the HI allele at the qhir8 HI-QTL as described herein. In some embodiments, the assay for identifying the HI allele at the qhir8 HI-QTL may comprise one of the genotyping methods described in Section II.E below. In some embodiments, the assay for identifying the HI allele at the qhir8 HI-QTL may be developed based on any of the known qhir8 HI-QTL markers. In some embodiments, the assay for identifying the HI allele at the qhir8 HI-QTL may be developed based on any difference between the wild-type sequence of the locus and the sequence of the variant allele of the locus associated with increased haploid induction.

In some embodiments, the assay to identify the HI allele at the qhir8 HI-QTL comprises genotyping an individual at one or more of the markers SM4849, SM8047, SM8133, SM8029, SM4257, and SM0956BQ. In some embodiments, the genotypes at these markers may be detected using a TaqMan real-time PCR assay (e.g., according to the methods detailed in Section E or in Example 1 herein). Table 2 lists expected genotypes and sequence contexts at each of these markers, according to some embodiments, along with example primers and probes which can be used in TaqMan real-time PCR genotyping assays.

TABLE 2
Exemplary markers used to genotype the qhir8 HI-QTL.

| Marker | SM4849 | SM8047 | SM8133 | SM8029 | SM4257 | SM0956BQ |
|---|---|---|---|---|---|---|
| Location (B73v5 | Chr 9 | Chr 9 | Chr 9 | Chr 9 | Chr 9 | Chr 9 |
| reference genome) | 3444422 | 3880187 | 3920009 | 4580317 | 7248352 | 11360090 |
| Wild-type genotype | G/G | A/A | G/G | G/G | C/C | A/A |
| and sequence context | SEQ ID | SEQ ID | SEQ ID | SEQ ID | SEQ ID | SEQ ID |
| | NO: 26 | NO: 32 | NO: 38 | NO: 44 | NO: 50 | NO: 56 |
| qhir8 HI allele | A/A | T/T | C/C | A/A | G/G | G/G |
| genotype and | SEQ ID | SEQ ID | SEQ ID | SEQ ID | SEQ ID | SEQ ID |
| sequence context | NO: 27 | NO: 33 | NO: 39 | NO: 45 | NO: 51 | NO: 57 |
| F primer | SEQ ID | SEQ ID | SEQ ID | SEQ ID | SEQ ID | SEQ ID |
| | NO: 22 | NO: 28 | NO: 34 | NO: 40 | NO: 46 | NO: 52 |
| R primer | SEQ ID | SEQ ID | SEQ ID | SEQ ID | SEQ ID | SEQ ID |
| | NO: 23 | NO: 29 | NO: 35 | NO: 41 | NO: 47 | NO: 53 |
| Probes | SEQ ID | SEQ ID | SEQ ID | SEQ ID | SEQ ID | SEQ ID |
| | NOS: 24-25 | NOS: 30-31 | NOS: 36-37 | NOS: 42-43 | NOS: 48-49 | NOS: 54-55 |

A/A: homozygous for adenine at marker; T/T: homozygous for thymine at marker; C/C: homozygous for cytosine at marker; G/G: homozygous for guanine at marker.

In some embodiments, the HI allele at the qhir8 HI-QTL comprises a variant allele at the DUF679 domain membrane protein 7 (DMP) gene (Zm00001d044822 in B73v5 reference genome>that is located within the qhir8 HI-QTL. See, e.g., Zhong, et al., 2019, “Mutation of ZmDMP enhances haploid induction in maize,” Nature Plants 5:575-580. In some embodiments, the maize plants are at least heterozygous (e.g., heterozygous or homozygous) for the HI variant allele at the DMP gene. In some embodiments, the variant allele is a loss-of-function mutation in DMP. Any assay that is able to identify a loss-of-function mutation in DMP may be used to identify the plants described herein. In some embodiments, the assay may comprise one of the genotyping methods described in Section E below. In some embodiments, the assay for identifying a loss-of-function mutation in DMP may be developed based on the wild-type sequence of the gene (SEQ ID NO:126).

In some embodiments, the assay for identifying a loss-of-function mutation in DMP may be a phenotypic assay. For example, levels of the protein encoded by DMP may be detected by any of a variety of methods known to those of skill in the art (e.g., western blot, immunofluorescence, mass spectrometry, etc.). In some embodiments, functional assays may be used to determine if the protein encoded by DMP is able to perform its usual function. For example, plants comprising putative DMP mutations may be crossed to tester plants to assess traits relevant to normal function of the protein encoded by DMP (e.g., seed set or haploid induction rate, as detailed in the Examples below).

In some embodiments, the maize plants described herein comprise at least one selectable marker to facilitate screening and selection of offspring of interest (e.g, offspring kernels that have become haploid). As used herein, the term selectable marker encompasses screening or reporter markers (e.g., color indicators that can be used to visually screen for offspring of interest) and selection markers (e.g., antibiotic resistance genes that can be used for antibiotic-mediated enrichment of offspring of interest). In some embodiments, the plants comprise a selectable marker gene. The selectable marker gene may be, for example, a mutation of an endogenous gene or a transgene. In some embodiments, the selectable marker gene encodes a detectable protein product. In some embodiments, the plants are heterozygous for a selectable marker. In some embodiments, the plants are homozygous for a selectable marker. In some embodiments, the selectable marker gene encodes a pigment or other detectable product that will only be present in diploid embryos, facilitating selection of haploid embryos, as detailed below and in the Examples. In some embodiments, the selectable marker may include any one of GUS, PMI, PAT, GFP, RFP, CFP, B1, CI, NPTII, HPT, ACC3, AADA, high oil content (see, e.g., Melchinger et al. 2013. Sci. Reports 3:2129 and Chaikam et al. 2019. Theor. and Appl. Genet. 132:3227-3243), R-navajo (R-nj), R1-scutellum (R1-SCM2), and/or an anthocyanin pigment. Other selectable marker genes are known to a person skilled in the art (see, e.g., Ziemienowicz. 2001. Acta Physiologiae Plantarum 23:363-374). In some embodiments, the selectable marker comprises an antibiotic resistance gene.

In some embodiments, the selectable marker comprises the R-navajo (“R-nj”) or R1-scutellum (“R1-SCM2”) variant alleles at the R1 locus on chromosome 10 (around position ˜139 Mb to ˜140 Mb in the B73v5 reference genome). These alleles confer a dominant anthocyanin trait that will express a purple or red color in both the embryo and endosperm of the seed. The R-nj allele is associated with very strong anthocyanin expression in the aleurone layer (outermost layer of the endosperm) and weaker expression in the embryo. The R1-SCM2 allele is associated with strong anthocyanin expression in the scutellum of the embryo and weaker expression in the aleurone layer. Diploid embryos resulting from a cross between a haploid inducer line comprising these dominantly expressed alleles and another line will display a purple or red color. Embryos which have lost the parental chromosome set of the haploid inducer line will not display the color.

Any assay that is able to distinguish between the wild-type R1 locus and the variant alleles R-nj and/or R1-SCM2 may be used to identify the plants described herein. In some embodiments, the assay may comprise one of the genotyping methods described in Section E below. In some embodiments, the assay may be developed based on the wild-type sequence of the locus and/or the sequence of the variant alleles R-nj and/or R1-SCM2.

In some embodiments, the assay to identify a plant comprising the variant R1-SCM2 allele at the R1 locus comprises genotyping an individual at one or more of the markers SM0954, SM0954HQ, SM6568, SM0953BQ, and SM6604. In some embodiments, the genotypes at these markers may be detected using a TaqMan real-time PCR assay (e.g., according to the methods detailed in Section E or in Example 1 herein). Table 3 lists expected genotypes and sequence contexts at each of these markers, according to some embodiments, along with example primers and probes which can be used in TaqMan real-time PCR genotyping assays.

TABLE 3
Exemplary markers used to genotype the R1 locus.

| Marker | SM0954BQ | SM0954HQ | SM6568 | SM0953BQ | SM6604 |
|---|---|---|---|---|---|
| Location (B73v5 | Chr 10 | Chr 10 | Chr 10 | Chr 10 | Chr 10 |
| reference genome) | 139300360 | 139300732 | 139789170 | 140146379 | 139789786 |
| Wild-type | A/A | A/A | A/A | G/G | A/A |
| genotype and | SEQ ID NO: 62 | SEQ ID NO: 68 | SEQ ID NO: 74 | SEQ ID NO: 80 | SEQ ID NO: 86 |
| sequence context | | | | | |
| R1-SCM2 | C/C | G/G | T/T | A/A | G/G |
| genotype and | SEQ ID NO: 63 | SEQ ID NO: 69 | SEQ ID NO: 75 | SEQ ID NO: 81 | SEQ ID NO: 87 |
| sequence context | | | | | |
| F primer | SEQ ID NO: 58 | SEQ ID NO: 64 | SEQ ID NO: 70 | SEQ ID NO: 76 | SEQ ID NO: 82 |
| R primer | SEQ ID NO: 59 | SEQ ID NO: 65 | SEQ ID NO: 71 | SEQ ID NO: 77 | SEQ ID NO: 83 |
| Probes | SEQ ID NOS: | SEQ ID NOS: | SEQ ID NOS: | SEQ ID NOS: | SEQ ID NOS: |
| | 60-61 | 66-67 | 72-73 | 78-79 | 84-85 |

A/A: homozygous for adenine at marker; T/T: homozygous for thymine at marker; C/C: homozygous for cytosine at marker; G/G: homozygous for guanine at marker.

In some embodiments, the maize plants described herein comprise a wild-type allele at a color inhibitor locus on chromosome 9 located between position 8 Mb and 10 Mb in the B73v5 reference genome. In some embodiments, the plants are at least heterozygous (e.g., heterozygous or homozygous) for a wild-type allele at the color inhibitor locus. A variant allele at this color inhibitor locus can reduce purple and/or red pigmentation in the embryo of a maize line comprising the R1-SCM2 allele at the R1 locus. The reduced pigmentation can make it more difficult to distinguish purple or red embryos (i.e., diploid embryos) from white or cream-colored embryos (i.e., haploid embryos). In some embodiments, selecting for maize plants that do not have this variant allele at the color inhibitor locus can ensure that the diploid offspring of said plants will display a strong purple or red color, making them easy to distinguish from white or cream-colored haploid embryos. In some embodiments, this selection is achieved by selecting for plants with a wild-type allele at the color inhibitor locus. Any assay that is able to distinguish between the wild-type color inhibitor locus and the variant allele may be used to identify the plants described herein. In some embodiments, the assay may comprise one of the genotyping methods described in Section E below. In some embodiments, the assay may be developed based on the wild-type sequence of the locus and/or the sequence of the variant color inhibitor allele.

In some embodiments, the assay to identify a wild-type allele at the chromosome 9 color inhibitor locus comprises genotyping an individual at one or both of the markers SM8040 and SM8091. In some embodiments, the genotypes at these markers may be detected using a TaqMan real-time PCR assay (e.g., according to the methods detailed in Section E or in Example 1 herein). Table 4 lists expected genotypes and sequence contexts at each of these markers, according to some embodiments, along with example primers and probes which can be used in TaqMan real-time PCR genotyping assays.

TABLE 4
Exemplary markers used to genotype the
chromosome 9 color inhibitor locus.

| Marker | SM8040 | SM8091 |
|---|---|---|
| Location (B73v5 | Chr 9 | Chr 9 |
| reference genome) | 8120045 | 10040065 |
| Wild-type | G/G | C/C |
| genotype and | SEQ ID NO: 92 | SEQ ID NO: 98 |
| sequence context | | |
| Color inhibitor | A/A | A/A |
| allele genotype and | SEQ ID NO: 93 | SEQ ID NO: 99 |
| sequence context | | |
| F primer | SEQ ID NO: 88 | SEQ ID NO: 94 |
| R primer | SEQ ID NO: 89 | SEQ ID NO: 95 |
| Probes | SEQ ID NOS: 90-91 | SEQ ID NOS: 96-97 |

A/A: homozygous for adenine at marker; C/C: homozygous for cytosine at marker; G/G: homozygous for guanine at marker.

### B. Methods of Determining the Haploid Induction Rate (HIR)

The haploid induction rate can be determined by harvesting test-crossed ears after pollination (e.g., at about 15 to 20 days after pollination). Embryos from the kernels can be isolated and incubated in appropriate media (referred to as embryo rescue media) suitable for maintaining the embryos' viability. In one embodiment, the rescue media used for HIR determination comprise 4.43 grams of Murashige and Skoog basal media with vitamins, 30 grams of sucrose, and 70 mg of salicylic acid. The embryos in the rescue media can be placed under a condition to allow the expression of the color indicator gene (e.g., R1-SCM2). In exemplary embodiments, the embryos are placed under 100-400 micromol light for 16-24 hours at 22-31° C. until some of the embryos turn purple due to the expression of the R1-SCM2 gene. See protocol, e.g., as described in WO2015/104358. The purple (diploid) and cream-colored (haploid) embryos can be counted from each ear. The frequency of haploids, known as the haploid induction rate, can be determined based on the number of haploids over the total embryos.

### C. Plant Transformability

The maize plants provided herein have a high level of transformability. Transformability can be measured in a variety of ways known to those of skill in the art. For example, as described in Example 1 below, embryos can be tested for transformability by transforming a test vector and detecting the percentage of embryos which are successfully transformed (i.e., transformation rate). Transformation, as used herein, can refer to any method of introducing foreign DNA into a maize genome (e.g., Agrobacterium-mediated transformation, particle bombardment, etc.). Example methods of maize transformation are described in Section IV below. In some embodiments, the maize plants provided herein display a transformation rate of at least 2%, at least 5%, at least 8%, at least 10%, at least 12%, or at least 15%. In some embodiments, maize plants provided herein with a high level of transformability have a normal A cytotype. In some embodiments, maize plants provided herein with a high level of transformability are at least heterozygous (i.e., heterozygous or homozygous) for a TF allele at at least one TF-QTL (e.g., the qCYTO-A_TF3.1 TF-QTL on chromosome 3). In some embodiments, the maize plants are homozygous for a TF allele at at least one TF-QTL (e.g., the qCYTO-A_TF3.1 TF-QTL). In some embodiments, maize plants that are homozygous for a TF allele at a TF-QTL have a higher level of transformability than maize plants that are heterozygous for the TF allele at the TF-QTL. In some embodiments, maize plants provided herein with a high level of transformability comprise both a normal A cytotype and a TF allele at at least one TF-QTL (e.g., the qCYTO-A_TF3.1 TF-QTL on chromosome 3). Maize plants comprising a TF allele at a TF-QTL may be identified using any known genotyping strategy, including those described herein.

In some embodiments, the plants and methods described herein comprise plants with a normal A (NA) cytotype. The cytotype of a plant can be determined via a variety of known methods. Any assay that is able to distinguish an NA cytotype from other known cytotypes, including normal B (NB) and cytoplasmic-male-sterile (CMS) cytotypes, may be used. In some embodiments, the assay may comprise one of the genotyping methods described in Section II.E below. In some embodiments, the assay for distinguishing an NA cytotype can be developed based on the NA and NB mitochondrial genomes disclosed by Allen, et al., 2007, “Comparisons among two fertile and three male-sterile mitochondrial genomes of maize,” Genetics 177: 1173-1192.

In some embodiments, the assay to distinguish an NA cytotype from other cytotypes comprises genotyping an individual at one or more of the markers SM2918, SM4813, SM2914, and SM4812. In some embodiments, the genotypes at these markers may be detected using a TaqMan real-time PCR assay (e.g., according to the methods detailed in Section II.E or in Example 1 herein). In some embodiments, one or both of the markers SM2918 and SM4813 are used to distinguish a normal cytotype (i.e., NA or NB) from a CMS cytotype. In some embodiments, one or both of the markers SM2914 and SM4812 are used to distinguish an NA cytotype from an NB cytotype. Table 5 lists expected genotypes and sequence contexts at each of these markers, according to some embodiments, along with example primers and probes which can be used in TaqMan real-time PCR genotyping assays.

TABLE 5
Exemplary markers used to distinguish normal A cytotype
from normal B cytotype and CMS cytotype individuals.

| Marker | SM2918 | SM4813 | SM2914 | SM4812 |
|---|---|---|---|---|
| Location | Mitochondrial genome | | | |
| NA cytotype | C/C | C/C | C/C | I/I |
| genotype and | SEQ ID NO: 104 | SEQ ID NO: 110 | SEQ ID NO: 116 | SEQ ID NO: 122 |
| sequence context | | | | |
| NB cytotype | C/C | C/C | A/A | D/D |
| genotype and | SEQ ID NO: 104 | SEQ ID NO: 110 | SEQ ID NO: 117 | SEQ ID NO: 123 |
| sequence context | | | | |
| CMS cytotype | A/A | A/A | Not applicable | Not applicable |
| genotype and | SEQ ID NO: 105 | SEQ ID NO: 111 | | |
| sequence context | | | | |
| F primer | SEQ ID NO: 100 | SEQ ID NO: 106 | SEQ ID NO: 112 | SEQ ID NO: 118 |
| R primer | SEQ ID NO: 101 | SEQ ID NO: 107 | SEQ ID NO: 113 | SEQ ID NO: 119 |
| Probes | SEQ ID NOS: | SEQ ID NOS: | SEQ ID NOS: | SEQ ID NOS: |
| | 102-103 | 108-109 | 114-115 | 120-121 |

C/C: homozygous for cytosine at marker; A/A: homozygous for adenine at marker; I/I: homozygous for 6 bp insertion allele at marker; D/D: homozygous for 6 bp deletion allele at marker.

In some embodiments, the maize plants disclosed herein comprise a TF allele at at least one quantitative trait locus (QTL) associated with increased transformability (TF-QTL). In some embodiments, the maize plants comprise a TF allele at the qCYTO-A_TF3.1 TF-QTL, located on chromosome 3 between position 14,742,407 and 70,562,070 in the B73v5 reference genome. Any assay that is able to identify or genotype a QTL may be used to identify the plants comprising the TF allele at the qCYTO-A_TF3.1 TF-QTL as described herein. In some embodiments, the TF allele at the qCYTO-A_TF3.1 TF-QTL matches that of the maize SYN-INBC34 line. In some embodiments, plants comprising a different allele at the TF-QTL (e.g., that of the maize RWKS/Z21S//RWKS line) are less amenable to transformation. In some embodiments, the assay for identifying the TF allele at the qCYTO-A_TF3.1 TF-QTL may comprise one of the genotyping methods described in Section II.E below. In some embodiments, the assay for identifying the TF allele at the qCYTO-A_TF3.1 TF-QTL may include genotyping a maize plant at any of the markers described herein (e.g., those described in Example 1 below and listed in Table 6, Table 22, and/or Table 23). In some embodiments, the assay for identifying the TF allele at the qCYTO-A_TF3.1 TF-QTL may be developed based on any difference between the sequence of the RWKS allele at the locus (i.e., a TF-QTL allele not associated with increased transformability) and the sequence of the SYN-INBC34 allele at the locus (i.e., the TF allele at the TF-QTL).

In some embodiments, the assay to identify the TF allele at the qCYTO-A_TF3.1 TF-QTL comprises genotyping an individual at one or more of the markers SM3158, SM4787, SM3814, SM3362, SM0634AQ, and SM4586. In some embodiments, the genotypes at these markers may be detected using a TaqMan real-time PCR assay (e.g., according to the methods detailed in Section II.E or in Example 1 herein). Table 6 lists expected genotypes and sequence contexts at each of these markers, according to some embodiments, along with example primers and probes which can be used in TaqMan real-time PCR genotyping assays.

TABLE 6
Exemplary markers used to genotype the qCYTO-A_TF3.1 TF-QTL.

| Marker | SM3158 | SM4787 | SM3814 | SM3362 | SM0634AQ | SM4586 |
|---|---|---|---|---|---|---|
| Location (B73v5 | Chr 3 | Chr 3 | Chr 3 | Chr 3 | Chr 3 | Chr 3 |
| reference genome) | 14742407 | 19210942 | 28710708 | 32750671 | 58719312 | 70562070 |
| RWKS genotype | A/A | A/A | A/A | A/A | C/C | A/A |
| and sequence | SEQ ID | SEQ ID | SEQ ID | SEQ ID | SEQ ID | SEQ ID |
| context | NO: 133 | NO: 139 | NO: 145 | NO: 151 | NO: 157 | NO: 163 |
| SYN-INBC34 | G/G | G/G | C/C | G/G | G/G | G/G |
| genotype and | SEQ ID | SEQ ID | SEQ ID | SEQ ID | SEQ ID | SEQ ID |
| sequence context | NO: 134 | NO: 140 | NO: 146 | NO: 152 | NO: 158 | NO: 164 |
| F primer | SEQ ID | SEQ ID | SEQ ID | SEQ ID | SEQ ID | SEQ ID |
| | NO: 129 | NO: 135 | NO: 141 | NO: 147 | NO: 153 | NO: 159 |
| R primer | SEQ ID | SEQ ID | SEQ ID | SEQ ID | SEQ ID | SEQ ID |
| | NO: 130 | NO: 136 | NO: 142 | NO: 148 | NO: 154 | NO: 160 |
| Probes | SEQ ID NOS: | SEQ ID NOS: | SEQ ID NOS: | SEQ ID NOS: | SEQ ID NOS: | SEQ ID NOS: |
| | 131-132 | 137-138 | 143-144 | 149-150 | 155-156 | 161-162 |

A/A: homozygous for adenine at marker; C/C: homozygous for cytosine at marker; G/G: homozygous for guanine at marker.

### D. Methods of Determining the Transformation Frequency (TF)

Transformation frequency (TF) can be determined using methods well known in the art. For example, a construct comprising one or more genes of interest can be introduced into a plant, a line of plants, or a plant cell using methods described in Section IV, below. The number of plants expressing the transgene over the number of plants or plant parts (e.g., embryos) one attempted to transform is calculated, which equals the TF. In some embodiments, the one or more transgenes of interest comprise an indicator transgene, the expression of which results in a phenotype that can be readily observed in plants. Thus, observation of the phenotype in the plant indicates a successful transformation.

### E. Methods of Genotyping

A variety of means can be used to genotype an individual (e.g., a plant) at a polymorphic site of interest such as a gene (e.g., MATL, DMP), a QTL (e.g., qhir8, qCYTO-A_TF3.1 chromosome 3 QTL), or a mitochondrial genome locus. In some embodiments, a genotyping assay is used to determine whether a sample (e.g., a nucleic acid sample) contains a specific variant allele (e.g., mutation or QTL marker) or haplotype. For example, enzymatic amplification of nucleic acid from an individual can be conveniently used to obtain nucleic acid for subsequent analysis. The presence or absence of a specific variant allele (e.g., mutation or QTL marker) or haplotype in one or more loci of interest can also be determined directly from the individual's nucleic acid without enzymatic amplification. In certain embodiments, an individual is genotyped at one, two, three, four, five, or more polymorphic sites such as a single nucleotide polymorphism (SNP) in one or more loci of interest. In some embodiments, an individual is genotyped at one, two, three, four, five, or more polymorphic sites in one or more loci of interest in the mitochondrial genome (e.g., to distinguish NA cytotype individuals from individuals of other cytotypes).

Genotyping of nucleic acid from an individual, whether amplified or not, can be performed using any of various techniques. Useful techniques include, without limitation, assays such as polymerase chain reaction (PCR) based analysis assays, sequence analysis assays, electrophoretic analysis assays, restriction length polymorphism analysis assays, hybridization analysis assays, allele-specific hybridization, oligonucleotide ligation allele-specific elongation/ligation, allele-specific amplification, single-base extension, molecular inversion probe, invasive cleavage, selective termination, restriction length polymorphism, sequencing, single strand conformation polymorphism (SSCP), single strand chain polymorphism, mismatch-cleaving, and denaturing gradient gel electrophoresis, all of which can be used alone or in combination.

Material containing nucleic acid is routinely obtained from individuals. Such material is any biological matter from which nucleic acid can be prepared. As a non-limiting example, material can be plant parts (e.g., leaves, stems, roots, flowers or flower parts, fruits, pollen, egg cells, zygotes, seeds, cuttings, cell or tissue cultures, or any other part or product of a plant) or any plant tissue or other plant part that comprises nucleic acid. In one embodiment, a method of the present disclosure is practiced with a leaf punch from a seedling, which can be obtained readily by non-invasive means and used to prepare genomic and/or mitochondrial DNA. In another embodiment, genotyping involves amplification of an individual's nucleic acid using the polymerase chain reaction (PCR).

Any of a variety of different primers can be used to amplify an individual's nucleic acid by PCR in order to determine the presence or absence of a variant allele (e.g., mutation or QTL marker) in a plant or method of the present disclosure. As understood by one skilled in the art, primers for PCR analysis can be designed based on the sequence flanking the polymorphic site(s) of interest in the gene of interest. As a non-limiting example, a sequence primer can contain from about 15 to about 30 nucleotides of a sequence upstream or downstream of the polymorphic site of interest in the gene or locus of interest. Such primers generally are designed to have sufficient guanine and cytosine content to attain a high melting temperature which allows for a stable annealing step in the amplification reaction. Several computer programs, such as Primer Select, are available to aid in the design of PCR primers.

An allelic discrimination assay (e.g., a TaqMan® assay available from Applied Biosystems) can be useful for genotyping an individual at a polymorphic site to thereby determine the presence or absence of a particular variant allele (e.g., mutation or QTL marker) or haplotype in the gene or locus of interest. In a TaqMan® allelic discrimination assay, a specific fluorescent dye-labeled probe for each allele is constructed. The probes contain different fluorescent reporter dyes such as FAM and TET to differentiate amplification of each allele. In addition, each probe has a quencher dye at one end which quenches fluorescence by fluorescence resonance energy transfer. During PCR, each probe anneals specifically to complementary sequences in the nucleic acid from the individual. The 5′ nuclease activity of Taq polymerase is used to cleave only probe that hybridizes to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Those skilled in the art understand that improved specificity in allelic discrimination assays can be achieved by conjugating a DNA minor groove binder (MGB) group to a DNA probe as described, e.g., in Kutyavin et al., Nuc. Acids Research 28:655-661 (2000). Minor groove binders include, but are not limited to, compounds such as dihydrocyclopyrroloindole tripeptide (DPI3).

Sequence analysis can also be useful for genotyping an individual according to the methods described herein to determine the presence or absence of a particular variant allele (e.g., mutation or QTL marker) or haplotype in the gene or locus of interest. As is known by those skilled in the art, a variant allele of interest can be detected by sequence analysis using the appropriate primers, which are designed based on the sequence flanking the polymorphic site of interest in the gene or locus of interest. For example, a variant allele in a gene or locus of interest can be detected by sequence analysis using primers designed by one of skill in the art. Additional or alternative sequence primers can contain from about 15 to about 30 nucleotides of a sequence that corresponds to a sequence about 40 to about 400 base pairs upstream or downstream of the polymorphic site of interest in the gene or locus of interest. Such primers are generally designed to have sufficient guanine and cytosine content to attain a high melting temperature which allows for a stable annealing step in the sequencing reaction. As used herein, the term “sequence analysis” includes any manual or automated process by which the order of nucleotides in a nucleic acid is determined, and encompasses, without limitation, chemical and enzymatic methods.

Electrophoretic analysis also can be useful in genotyping an individual according to the methods of the present disclosure to determine the presence or absence of a particular variant allele (e.g., mutation or QTL marker) or haplotype in the gene or locus of interest. “Electrophoretic analysis” as used herein in reference to one or more nucleic acids such as amplified fragments includes a process whereby charged molecules are moved through a stationary medium under the influence of an electric field. Methods of electrophoretic analysis, and variations thereof, are well known in the art, as described, for example, in Ausubel et al., Current Protocols in Molecular Biology Chapter 2 (Supplement 45) John Wiley & Sons, Inc. New York (1999).

Restriction fragment length polymorphism (RFLP) analysis can also be useful for genotyping an individual according to the methods of the present disclosure to determine the presence or absence of a particular variant allele (e.g., mutation or QTL marker) or haplotype in the gene or locus of interest (see, Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). RFLP analysis may be performed on PCR amplification products.

In addition, allele-specific oligonucleotide hybridization can be useful for genotyping an individual in the plants or methods described herein to determine the presence or absence of a particular variant allele (e.g., mutation or QTL marker) or haplotype in the gene or locus of interest. Allele-specific oligonucleotide hybridization is based on the use of a labeled oligonucleotide probe having a sequence perfectly complementary, for example, to the sequence encompassing the variant allele. Under appropriate conditions, the variant allele-specific probe hybridizes to a nucleic acid containing the variant allele but does not hybridize to the one or more other alleles, which have one or more nucleotide mismatches as compared to the probe. If desired, a second allele-specific oligonucleotide probe that matches an alternate (e.g., wild-type) allele can also be used. Similarly, the technique of allele-specific oligonucleotide amplification can be used to selectively amplify, for example, a variant allele by using an allele-specific oligonucleotide primer that is perfectly complementary to the nucleotide sequence of the variant allele but which has one or more mismatches as compared to other alleles (Mullis et al., supra). One skilled in the art understands that the one or more nucleotide mismatches that distinguish between the variant allele and other alleles are often located in the center of an allele-specific oligonucleotide primer to be used in the allele-specific oligonucleotide hybridization. In contrast, an allele-specific oligonucleotide primer to be used in PCR amplification generally contains the one or more nucleotide mismatches that distinguish between the variant and other alleles at the 3′ end of the primer.

A heteroduplex mobility assay (HMA) is another well-known assay that can be used for genotyping in the plants or methods of the present disclosure to determine the presence or absence of a particular variant allele (e.g., mutation or QTL marker) or haplotype in the gene or locus of interest. HMA is useful for detecting the presence of a variant allele since a DNA duplex carrying a mismatch has reduced mobility in a polyacrylamide gel compared to the mobility of a perfectly base-paired duplex (see, Delwart et al., Science, 262:1257-1261 (1993); White et al., Genomics, 12:301-306 (1992)).

The technique of single strand conformational polymorphism (SSCP) can also be useful for genotyping in the plants or methods described herein to determine the presence or absence of a particular variant allele (e.g., mutation or QTL marker) or haplotype in the gene or locus of interest (see, Hayashi, Methods Applic., 1:34-38 (1991)). This technique is used to detect variant alleles based on differences in the secondary structure of single-stranded DNA that produce an altered electrophoretic mobility upon non-denaturing gel electrophoresis. Variant alleles are detected by comparison of the electrophoretic pattern of the test fragment to corresponding standard fragments containing known alleles.

Denaturing gradient gel electrophoresis (DGGE) can also be useful in the plants or methods of the present disclosure to determine the presence or absence of a particular variant allele (e.g., mutation or QTL marker) or haplotype in the gene or locus of interest. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (see, Sheffield et al., “Identifying DNA Polymorphisms by Denaturing Gradient Gel Electrophoresis” in Innis et al., supra, 1990).

Other molecular methods useful for genotyping an individual are known in the art and useful in the plants or methods of the present disclosure. Such well-known genotyping approaches include, without limitation, automated sequencing and RNase mismatch techniques (see, Winter et al., Proc. Natl. Acad. Sci., 82:7575-7579 (1985)). Furthermore, one skilled in the art understands that, where the presence or absence of multiple variant alleles is to be determined, individual variant alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple variant alleles can be detected in individual reactions or in a single reaction (a “multiplex” assay).

### F. DNA Modification Enzymes

In some embodiments, the HI-NA maize plants of the present disclosure are capable of expressing a DNA modification enzyme. In some embodiments, such plants are optionally also able to express at least one guide nucleic acid (e.g., guide RNA). In some embodiments, the DNA modification is a site-directed nuclease selected from the group consisting of Cas9 nuclease, Cas12a nuclease, meganucleases (MNs), zinc-finger nucleases, (ZFNs), transcription-activator like effector nucleases (TALENs), dCas9-Fokl, dCas12a-FokI, chimeric Cas9-cytidine deaminase, chimeric Cas9-adenine deaminase, chimeric FENI-FokI, MegaTALs, a nickase Cas9 (nCas9), chimeric dCas9 non-Fokl nuclease, dCas12a non-Fokl nuclease, chimeric Cas12a-cytidine deaminase, and Cas12a-adenine deaminase. Methods for obtaining such plants are described in more detail in Section III below.

### G. Plant Heterotic Groups

Advantageously, the maize plants of the present disclosure, including the HI plants and NA plants used in the breedings to produce the HI-NA plants discussed herein, may derive from any known heterotic group. Aside from trait introgression, a goal of plant breeding is to make genetic improvements in varietal lines and also parental lines of hybrids. An effective hybrid breeding program makes genetic improvements to parent lines in both the hybrid's female parent heterotic group and the hybrid's male parent heterotic group. Therefore, it is advantageous to make genetic improvements in all heterotic groups used in a breeding program. Table 7 shows the common heterotic groups to which various germplasms belong. The HI-NA plants can also be used to cross with a maize plant from any heterotic group to edit its genome and improve its traits. In some embodiments, the maize plants of the present disclosure belong to any of the heterotic groups in Table 7. In some embodiments, the maize plant comprises a Stiff Stalk germplasm, a Non-Stiff Stalk germplasm, a Non-Stiff Stalk Iodent germplasm, a tropical germplasm, or a subtropical germplasm. In other embodiments, the maize plant comprises a germplasm classified into any other heterotic group known to one of skill in the art (see, e.g., L. Reid, et al., 2011, “Genetic diversity analysis of 119 Canadian maize inbred lines based on pedigree and simple sequence repeat markers,” Can. J. Plant Sci. 91: 651-661 and M. Mikel and J. Dudley, 2006, “Evolution of North American Dent Corn from Public to Proprietary Germplasm,” Crop Sci. 46: 1193-1205, each of which is incorporated herein by reference in its entirety). The maize plants of the present disclosure may also be derived from any publically known or proprietary line. In some embodiments, the maize plant is derived from any of lines Stock 6, RWK, RWS, UH400, AX5707RS, and/or NP2222. In other embodiments, the maize plant is derived from any other line of interest.

TABLE 7
Heterotic groups and example germplasm lines.

| Heterotic group | Germplasm lines |
|---|---|
| Non-Stiff Stalk | SYN-INBE56, SYN-INBB23 |
| Stiff Stalk | NP2222, SYN-INBF67, SYN-INBC34 |
| Non-Stiff Stalk Iodent | SYN-INBD45, SYN-INBG78, SYN- |
| | INBH89, SYN-INBI90 |
| Non-Stiff Stalk Mo17-like | SYN-INBJ13 |
| Tropical | SYN-INBK14 |

### Iii. Production of HI-NA Plants

In another aspect, provided herein are methods of producing transformable haploid inducer maize plants (HI-NA plants). In some embodiments, production of HI-NA plants involves crossing a HI plant line (possessing a combination of HI alleles at any of the genes or HI-QTLs as disclosed above) as a pollen donor with a NA plant line as the recipient. In some embodiments, the recipient plant line also comprises a TF allele at one or more TF-QTLs as described above. In some embodiments, the recipient plant line comprises a TF allele at the qCYTO-A_TF3.1 TF-QTL on chromosome 3. In some embodiments, the pollen donor plant line and/or the recipient plant line exhibit high pollen load and/or tassel weight. In some embodiments, wild-type alleles are modified to form the corresponding HI alleles at one or more genes and/or HI-QTLs. In some embodiments, the gene editing machinery for editing all of the HI alleles and HI-QTLs are delivered in the same target plant, e.g., through a co-transformation process. In some embodiments, editing of HI-QTLs/HI alleles occurs sequentially. For example, the gene editing machinery for editing the wild type allele to produce the first HI allele (e.g., at the qhir8 QTL) may be delivered first, and plants comprising the first HI allele are selected. Subsequently, the gene-editing machinery targeting the second HI allele (e.g., at the MATL gene) is introduced to the same plant or subsequent generations of the same plant that already comprises the first HI allele, and so on. In some embodiments, two or more HI-QTL/HI alleles are simultaneously edited, followed by editing additional HI-QTL/HI alleles. Various alternatives of the aforementioned transformation schemes are also contemplated and encompassed in this disclosure. Exemplary embodiments of the production of HI plants are disclosed in U.S. Pat. No. 10,285,348, the entire disclosure of which is herein incorporated by reference.

### A. Breeding Strategies

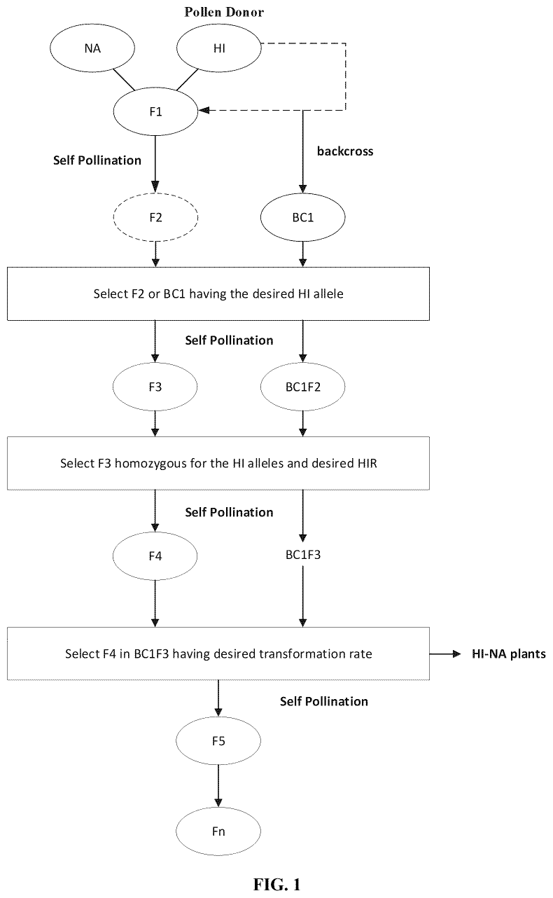

Various methods can be used to produce the HI-NA plants in this disclosure. One exemplary embodiment of the methods is illustrated in FIG. 1. In some embodiments, the HI plants can serve as a pollen donor parent (male parent) and be crossed with the NA plants as the female parent to generate F1 plants. In some embodiments, the pollen donor parent is homozygous for a loss-of-function mutation in the MATL gene and is at least heterozygous (e.g., heterozygous or homozygous) for a HI allele at a second locus. In some embodiments, the HI allele comprises a HI allele at the qhir8 HI-QTL. In some embodiments, the HI allele comprises a loss-of-function mutation in the DMP gene within the qhir8 HI-QTL. In some embodiments, the pollen donor parent is transformation recalcitrant. In some embodiments, the female parent maize plant is at least heterozygous (e.g, heterozygous or homozygous) for a TF allele at a third locus (e.g., at a TF-QTL). In some embodiments, the female parent is at least heterozygous for a TF allele at the qCYTO-A_TF3.1 TF-QTL on chromosome 3. In some embodiments, pollen from the pollen donor parent is used to pollinate the female parent maize plant.

In some embodiments, the F1 progeny plants from the crosses described above will all have NA cytoplasm due to maternal cytoplasmic inheritance. In some embodiments, the F1 progeny plants will be at least heterozygous for a TF allele. In some embodiments, the pollen donor HI plant carries an allele for a selectable marker, as described above, to allow differentiation between haploid and diploid progeny embryos. In some embodiments, the pollen donor HI plant is homozygous for the selectable marker. In some embodiments, the selectable marker may include any one of GUS, PMI, PAT, GFP, RFP, CFP, B1, CI, NPTII, HPT, ACC3, AADA, high oil content, R-navajo (R-nj), R1-scutellum (R1-SCM2), and/or an anthocyanin pigment. In some embodiments, the selectable marker is the R1-SCM2 allele at the R1 locus on chromosome 10. In some embodiments, the pollen donor HI plant comprising the R1-SCM2 allele is also at least heterozygous for a wild-type allele at the color inhibitor locus on chromosome 9, as described above. Diploid embryos that are heterozygous or homozygous for the R1-SCM2 allele will exhibit a purple color, while haploid embryos that do not have the R1-SCM2 allele will exhibit a cream color. Since the color indicator gene is from the same parent as the HI alleles, the color of an embryo may indicate whether it carries the HI alleles. In some embodiments, an embryo that is purple in color is diploid.

In some embodiments, diploid F1 plants are selected and self-pollinated to produce embryos for the F2 generation. In other embodiments, the diploid F1 plants are backcrossed with the NA parent plant line to produce the BC1 generation. In some embodiments, the diploid F1 plants are identified using the selectable marker. In some embodiments, those that have the selectable marker product (e.g., those that have a purple color in the case of the R1-SCM2 allele) also carry the HI alleles. The F2 plants and the BC1 plants may be genotyped to confirm the presence of the HI alleles using methods as described above. In some embodiments, F2 and/or BC1 plants that are either homozygous or heterozygous for the HI alleles as described above are selected for further breeding. In some embodiments, the F2 and/or BC1 progeny plants are selected for NA cytotype plants that are homozygous for the loss-of-function mutation in the MATL gene and at least heterozygous for the additional HI allele (e.g., at the qhir8 HI-QTL). In some embodiments, the F2 and/or BC1 progeny plants are selected for plants that are at least heterozygous for a TF allele (e.g., at the qCYTO-A_TF3.1 TF-QTL on chromosome 3).

In some embodiments, the selected F2 plants are self-pollinated to produce F3 plants. In some embodiments, the selected BC1 plants are self-pollinated to generate BC1F2 plants. The F3 plants and/or the BC1F2 plants may be genotyped for the presence of the HI alleles, the TF alleles, and/or the the selectable marker. In some embodiments, these plants are also tested for HIR using methods disclosed herein. Suitable F3 plants and/or BC1F2 plants may be selected for further breeding if they are at least heterozygous for the desired HI allele combination and also have an HIR at least 5%, at least 6%, at least 7%, or at least 10%.

The selected F3 plants and/or BC1F2 plants may be self-pollinated to generate the F4 plants and/or BC1F3 plants in a similar manner. In some embodiments, HIR and transformation frequency (TF) of these plants are determined. In some embodiments, the plants exhibiting a sufficiently high HIR, for example, at least 10%, at least 12%, or at least 15%, and also a high TF, for example, at least 2%, at least 5%, at least 7%, at least 9%, at least 10%, at least 15%, at least 40%, at least 50%, or at least 60% are selected as the HI-NA plants.

In some embodiments, the F4 plants and/or the BC1F3 plants that exhibit sufficiently high HIR are further bred through self-pollination to produce further generations of plants, e.g., F5, F6, F7, BC1F4, BC1F5, BC1F6. These plants may be tested to confirm that they have the desired HIR and TF rate.

Additional embodiments of the breeding strategies described above are also contemplated herein. For example, the BC1 plants described above may be backcrossed to the NA parent line to generate BC2 plants. In some embodiments, each generation is repeatedly backcrossed to a parent line (e.g., to generate BC3 plants, BC4 plants, BC5 plants, etc.). In some embodiments, self-pollination crosses are performed after one or more backcross generations (e.g., to generate BC2F2 plants, BC2F3 plants, BC3F2 plants, BC2F3 plants, etc.). In some embodiments, one or more of the genotyping for HI alleles, genotyping for TF alleles, phenotyping for HIR and/or TF, etc., may be performed at each generation.

In some embodiments, the male parent and/or the female parent of the methods described above belong to any of the heterotic groups in Table 7. In some embodiments, the male parent belongs to a different heterotic group than the female parent. In some embodiments, the male parent and/or the female parent comprises a Stiff Stalk germplasm, a Non-Stiff Stalk germplasm, a Non-Stiff Stalk Iodent germplasm, a tropical germplasm, or a subtropical germplasm. In other embodiments, the male parent and/or the female parent comprises a germplasm classified into any other heterotic group known to one of skill in the art (see, e.g., L. Reid, et al., 2011, “Genetic diversity analysis of 119 Canadian maize inbred lines based on pedigree and simple sequence repeat markers,” Can. J. Plant Sci. 91: 651-661 and M. Mikel and J. Dudley, 2006, “Evolution of North American Dent Corn from Public to Proprietary Germplasm,” Crop Sci. 46: 1193-1205, each of which is incorporated herein by reference in its entirety). The male parent and/or the female parent may also be derived from any publically known or proprietary line. In some embodiments, the male parent and/or the female parent is derived from any of lines Stock 6, RWK, RWS, UH400, AX5707RS, and/or NP2222.

### B. Breeding and Mutational Targeting

In another aspect, methods to produce the transformable haploid inducer maize plants described herein comprise a combination of trait introgression via breeding and direct mutational targeting of genes. In some embodiments, wild-type alleles are modified to form HI alleles. In some embodiments, mutational targeting of the MATL and/or DMP genes is used to produce HI alleles (e.g., loss-of-function mutations matl and/or dmp). In some embodiments, mutational targeting is achieved via gene editing. In some embodiments, the gene editing machinery for editing all of the HI alleles are delivered in the same target plant, e.g., by guide RNA multiplexing or through a co-transformation process. In some embodiments, editing of HI alleles occurs sequentially. For example, the gene editing machinery for editing the wild type allele to produce the first HI allele (e.g., a loss-of-function mutation in DMP) may be delivered first, and plants stably expressing the first HI allele are selected. Subsequently, the gene-editing machinery targeting the second HI allele (e.g., a loss-of-function mutation in MATL) is introduced to the same plant or subsequent generations of the same plant that already expresses the first HI allele, and so on. In some embodiments, two or more HI alleles are simultaneously edited, followed by editing additional HI alleles. Various alternatives of the aforementioned transformation schemes are also contemplated and encompassed in this disclosure. Exemplary embodiments of the production of HI plants are disclosed in U.S. Pat. No. 10,285,348, the entire disclosure of which is herein incorporated by reference.

In some embodiments, a maize plant comprising wild-type alleles of the MATL and DMP genes is used as a pollen donor (i.e., the male parent plant) in a cross with another maize plant (i.e., the female parent plant). In some embodiments, the female parent plant comprises an NA cytoplasm. In some embodiments, the female parent plant is at least heterozygous (e.g., heterozygous or homozygous) for a TF allele at a TF-QTL (e.g., at the qCYTO-A_TF3.1 TF-QTL on chromosome 3). In some embodiments, the pollen donor plant carries a selectable marker gene allele, as described above, to allow differentiation between haploid and diploid progeny embryos. In some embodiments, the pollen donor plant is at least heterozygous for the selectable marker gene allele. In some embodiments, the selectable marker gene allele may include any one of GUS, PMI, PAT, GFP, RFP, CFP, B1, CI, NPTII, HPT, ACC3, AADA, high oil content, R-navajo (R-nj), R1-scutellum (R1-SCM2), and/or an anthocyanin pigment. In some embodiments, the selectable marker gene allele is the R1-SCM2 allele at the R1 locus on chromosome 10. In some embodiments, the pollen donor plant comprising the R1-SCM2 allele is also at least heterozygous for a wild-type allele at the color inhibitor locus on chromosome 9, as described above.

In some embodiments, F1 plants are self-pollinated to produce embryos for the F2 generation. In other embodiments, F1 plants are backcrossed with the female or male parent plant line to produce the BC1 generation. The F2 plants and/or the BC1 plants may be selected for the presence of an NA cytoplasm, the TF allele at the TF-QTL, the selectable marker gene allele, and/or the wild-type allele at the color inhibitor locus on chromosome 9 using genotyping methods as described above. The selected F2 and/or BC1 plants may be self-pollinated and/or backcrossed for one or several more generations, as described above. In some embodiments, the selected F2 and/or BC1 plants, or progeny therefrom, are edited, as described above and in Section V below, to cause a loss-of-function mutation in the MATL gene and/or the DMP gene to obtain a transformable haploid inducer plant. In some embodiments, the transformable haploid inducer plant is self-pollinated and/or backcrossed for one or several generations. The transformable haploid inducer plant, or progeny therefrom, may be genotyped and/or phenotyped as described above to select a plant having high transformability and high haploid induction.

In some embodiments, the male parent and/or the female parent of the methods described above belong to any of the heterotic groups in Table 7. In some embodiments, the male parent belongs to a different heterotic group than the female parent. In some embodiments, the male parent and/or the female parent comprises a Stiff Stalk germplasm, a Non-Stiff Stalk germplasm, a Non-Stiff Stalk Iodent germplasm, a tropical germplasm, or a subtropical germplasm. In other embodiments, the male parent and/or the female parent comprises a germplasm classified into any other heterotic group known to one of skill in the art (see, e.g., L. Reid, et al., 2011, “Genetic diversity analysis of 119 Canadian maize inbred lines based on pedigree and simple sequence repeat markers,” Can. J. Plant Sci. 91: 651-661 and M. Mikel and J. Dudley, 2006, “Evolution of North American Dent Corn from Public to Proprietary Germplasm,” Crop Sci. 46: 1193-1205, each of which is incorporated herein by reference in its entirety). The male parent and/or the female parent may also be derived from any publically known or proprietary line. In some embodiments, the male parent and/or the female parent is derived from any of lines Stock 6, RWK, RWS, UH400, AX5707RS, and/or NP2222.

### Iv. Transformation of HI-NA Maize Plants

In another aspect, provided herein are methods for transforming a HI-NA plant described above. In some embodiments, a gene of interest (e.g., a gene encoding a DNA modification enzyme as disclosed above and one or more guide RNAs,) can be introduced into the HI-NA maize plants described above. Suitable methods for the transformation of plants are protoplast transformation by polyethylene glycol-induced DNA uptake, the biolistic process using the gene gun-the “particle bombardment” method, cell-penetrating peptide (CPP)-mediated transformation, glycol mediated transformation, electroporation, microinjection and gene transfer, described above, mediated by Agrobacterium. The processes mentioned are described, for example, in B. Jenes et al., Techniques for Gene Transfer, in: Transgenic Plants, Vol. 1, Engineering and Utilization, edited by S. D. Kung and R. Wu, Academic Press (1993), 128-143 and in Potrykus, Annu. Rev. Plant Physiol. Plant Molec. Biol. 42 (1991), 205-225).

In one embodiment, the gene of interest is cloned in a vector which is suitable to be transformed in Agrobacterium tumefaciens. Agrobacteria transformed using such a vector can then be used in a known manner for the transformation of plants, in particular of crop plants, by, for example, bathing wounded leaves or pieces of leaf in an Agrobacteria solution and subsequently culturing in suitable media.

In some embodiments, one or more genes known to have the capability to increase transformability are co-transformed with the gene of interest into the HI-NA plant. These genes are referred to as morphogenic factors or booster genes in this application. Classes of morphogeneic factors include BABY BOOM (BBM), BBM-like, EMBRYOMAKER (EMK), AINTEGUMENTA (ANT), AINTEGUMENTA-LIKE (AIL), PLETHORA (PLT), WUSCHEL (WUS) or WUS homeobox (Wox), GRF (Growth Regulating Factor), SHOOT MERISTEMLESS (STM), AGAMOUS-Like (AGL), MYB115, MYB118, Somatic embryogenesis receptor-like kinase (SERK), SOMATIC EMBRYO RELATED FACTOR (SERF) and AT-HOOK MOTIF CONTAINING NUCLEAR LOCALIZED (AHL). Non-limiting examples of the booster genes include OVULE DEVELOPMENT PEPTIDE (ODP), BABY BOOM (BBM), WUSCHEL2 (WUS2), WUSCHEL-RELATED HOMEOBOX 5 (WOX5), GROWTH-REGULATING FACTOR 5 (GRF5), or a chimeric protein combining GROWTH-REGULATING FACTOR 4 (GRF4) and its cofactor GRF-INTERACTING FACTOR 1 (GIF1). Methods of performing the co-transformation of the booster gene with a gene of interest to boost transformation efficiency are known, for example as disclosed in Lowe et al. (2016) “Morphogenic Regulators Baby boom and Wuschel Improve Monocot Transformation”, Plant Cell 28, 1998-2015; Hoerster, et al. (2020) “Use of non-integrating ZmWus2 vectors to enhance maize transformation”, In Vitro Cellular & Developmental Biology; Mookkan et al., (2017) “Selectable marker independent transformation of recalcitrant maize inbred B73 and sorghum P898012 mediated by morphogenic regulators BABYBOOM and WUSCHEL2”, Plant Cell Reports 36:1477-1491; Kong et al. (2020) “Overexpression of Transcription Factor Growth Regulating Factor5 Improves Transformation of Monocot and Dicot Species”, Front. in Plant Sci, 10.3389/fpls.2020.572319), or by utilizing GRF4-GIF1 as described in Debernardi, J. M. et al. (2020), “A GRF-GIF chimeric protein improves the regeneration efficiency of transgenic plants”, Nature Biotechnology 38: 1274-1279. The entire contents of the aforementioned references are herein incorporated by reference. See also U.S. Pat. Nos. 7,151,170; 7,579,529; 7,256,322; 7,700,829; WO 2018/224001; WO 2018/098420; and PCT/US2020/45573; all of which the contents thereof are incorporated herein by reference in their entireties.

In some embodiments, a booster gene used in the co-transformation resides in a different vector from the gene of interest. In some embodiments, the booster gene is in the same vector as the gene of interest. Examples 2 and 7 below show illustrative exemplary embodiments of the co-transformation of one or more booster genes with the gene of interest into the HI-NA plants disclosed herein.

### V. Gene Editing of Target Plants

Various embodiments of the methods described herein use gene editing. In some embodiments, gene editing is used to mutagenize the genome of a plant to produce plants having one or more HI alleles (e.g., a HI allele of a gene and/or a HI allele at a HI-QTL) and/or one or more TF alleles (e.g., at a TF-QTL).

In some embodiments, provided herein are HI-NA plants transformed with and expressing gene-editing machinery as described above, which, when crossed with a target plant, result in gene editing in the target plant.

In general, gene editing may involve transient, inducible, or constitutive expression of the gene editing components or systems. Gene editing may involve genomic integration or episomal presence of the gene editing components or systems.

Gene editing generally refers to the use of a site-directed nuclease (including but not limited to CRISPR/Cas, zinc fingers, meganucleases, and the like) to cut a nucleotide sequence at a desired location. This may be to cause an insertion/deletion (“indel”) mutation, (i.e., “SDN1”), a base edit (i.e., “SDN2”), or allele insertion or replacement (i.e., “SDN3”). SDN2 or SDN3 gene editing may comprise the provision of one or more recombination templates (e.g., in a vector) comprising a gene sequence of interest that can be used for homology directed repair (HDR) within the plant (i.e. to be introduced into the plant genome). In some embodiments, the gene of interest may be a HI allele (e.g., matl or dmp) to be introduced into a plant genome to generate a HI plant or HI-NA plant. In some embodiments, the gene or allele of interest is one that is able to confer to the plant an improved trait, e.g., improved yield. The recombination template can be introduced into the plant to be edited either through transformation or through breeding with a donor plant comprising the recombination template. Breaks in the plant genome may be introduced within, upstream, and/or downstream of a target sequence. In some embodiments, a double strand DNA break is made within or near the target sequence locus. In some embodiments, breaks are made upstream and downstream of the target sequence locus, which may lead to its excision from the genome. In some embodiments, one or more single strand DNA breaks (nicks) are made within, upstream, and/or downstream of the target sequence (e.g., using a nickase Cas9 variant). Any of these DNA breaks, as well as those introduced via other methods known to one of skill in the art, may induce HDR. Through HDR, the target sequence is replaced by the sequence of the provided recombination template. In certain embodiments, a gene sequence of interest as described herein (e.g., the matl or dmp allele sequences) may be provided on/as a template. By designing the system such that one or more single strand or double strand breaks are introduced within, upstream, and/or downstream of the corresponding region in the genome of a plant not comprising the gene sequence of interest, this region can be replaced with the template comprising the gene sequence of interest. In this way, introduction of the gene sequence of interest in a plant need not involve multiple backcrossing, in particular in a plant of specific genetic background. Similarly, a mutated gene sequence of interest (e.g., matl or dmp) may be provided as a template.

In some embodiments, mutations in the genes of interest described herein (e.g., matl or dmp) may be generated without the use of a recombination template via targeted introduction of DNA double strand breaks. Such breaks may be repaired through the process of non-homologous end joining (NHEJ), which can result in the generation of small insertions or deletions (indels) at the repair site. Such indels may lead to frameshift mutations causing premature stop codons or other types of loss-of-function mutations in the targeted genes.

In some embodiments, gene editing may involve transient, inducible, or constitutive expression of the gene editing components or systems in the target plant. Gene editing may also involve genomic integration or episomal presence of the gene editing components or systems in the target plant.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261; 6,607,882; 6,746,838; 6,794,136; 6,824,978; 6,866,997; 6,933,113; and 6,979,539.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a“spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cas12a).

The Cas protein as referred to herein, such as without limitation Cas9, Cas12a (formerly referred to as Cpf1), Cas12b (formerly referred to as C2c1), Cas13a (formerly referred to as C2c2), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is Cas12a, optionally from Acidaminococcus sp., such as Acidaminococcus sp. BV3L6 Cpf1 (AsCas12a) or Lachnospiraceae bacterium Cas12a, such as Lachnospiraceae bacterium MA2020 or Lachnospiraceae bacterium MD2006 (LBCas12a). See U.S. Pat. No. 10,669,540, incorporated herein by reference in its entirety. Alternatively, the Cas12a protein may be from Moraxella bovoculi AAX08_00205 [Mb2Cas12a]or Moraxella bovoculi AAX11_00205 [Mb3Cas12a]. See WO 2017/189308, incorporated herein by reference in its entirety. In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2cl. In certain embodiments, the (modified) Cas protein is C2c3. In certain embodiments, the (modified) Cas protein is Cas13b. Other Cas enzymes are available to a person skilled in the art.

The gene-editing machinery (e.g., the DNA modifying enzyme) introduced into the plants (e.g., the HI-NA plants) can be controlled by any promoter that can drive recombinant gene expression in maize. In some embodiments, the promoter is a constitutive promoter. In some embodiments, the promoter is a tissue-specific promoter, e.g., a pollen-specific promoter or a sperm cell specific promoter, a zygote specific promoter, or a promoter that is highly expressed in sperm, eggs and zygotes (e.g., prOsActin1). Suitable promoters are disclosed in U.S. Pat. No. 10,519,456, the entire content of which is herein incorporated by reference. Exemplary promoters are shown in Table 8 below.

TABLE 8
List of suitable promoters to drive the
expression of the editing machinery in plants.
Promoters List: promoters one can use in a transgene
to drive high sperm cell expression of editing
machinery to boost the efficiency of simultaneous
editing and doubled-haploid induction (“SEDHI”).

| Gene Name | Gene ID | Maize Ortholog | Rice Ortholog |
|---|---|---|---|
| DUO1 | At3G60460 | GRMZM2G105137, | LOC_Os04g46384 |
| | | GRMZM2G046443 | |
| MGH3 | At1G19890 | NA | NA |
| GEX1 | At5G55490 | GRMZM2G388045 | LOC_Os09g27040, |
| | | | LOC_Os07g47194 |
| GEX2 | At5G49150 | GRMZM2G036832 | LOC_Os09g25650 |
| GEX3 | At5G16020 | GRMZM2G458159 | LOC_Os01g42060 |
| HAP2/GSC1 | At4G11720 | GRMZM2G412911 | LOC_Os05g18730 |
| CycB1 | At4G37490 | NA | NA |
| DAZ1 | At2G17180 | GRMZM2G132057 | NA |
| DAZ2 | At4G35280 | NA | LOC_Os02g19180 |
| DAZ3 | At4G35700 | NA | NA |
| PCR11 | At1G68610 | NA | NA |
| DAN1 | At3G04620 | NA | NA |
| TIP1 | AT3G47440 | NA | LOC_Os04g46490 |
| MKKK20 | AT3G50310 | NA | NA |
| DAF1 | At3G62230 | NA | NA |
| DAW1 | At4G35560 | GRMZM2G176647 | NA |
| DAU2/DMP9 | At5G39650 | NA | NA |
| prOsActin1 | | | Os03g0718150 |

In another aspect, provided herein is a method of editing plant genomic DNA. In some embodiments, the method comprises use of a HI-NA maize plant expressing a DNA modification enzyme and at least one optional guide nucleic acid as described above to pollinate a target plant comprising genomic DNA to be edited. In some embodiments, at least one haploid progeny from the cross is selected. In some embodiments, the haploid progeny comprises the genome of the target plant and does not comprise the genome of the HI-NA maize plant. In some embodiments, the haploid progeny does not express the DNA modification enzyme. In some embodiments, the genome of the haploid progeny has been modified by the DNA modification enzyme and optional guide nucleic acid delivered by the HI-NA maize plant. This process, known as HI-Edit, is described in U.S. Pat. Nos. 10,519,456 10,285,348. The edited haploid plants can then be identified and treated with a doubling agent, thereby creating an edited doubled haploid progeny. Non-limiting examples of the chromosome doubling agents include colchicine, pronamide, dthipyr, triflualin, or another known anti-microtubule agent. The diploid plants are then grown to maturity and self-pollinated to generate edited diploid seed, which will be used for additional breeding and seed production processes. Optionally, all diploid generation lines can be evaluated to confirm the existence of homozygous target-site edits and the lack of the gene editing machinery.

### EXAMPLES

### Example 1. Breeding Transformable Haploid Inducers

### Maize Lines and Genotyping Markers

A transformable haploid inducer line can be bred by crossing together a haploid inducer line and a transformable line. It is preferable to use a transformable line that has a “normal A” cytotype (i.e., has a C/C genotype for markers SM2918, and/or SM4813, and SM2914, and/or an I/I genotype for marker SM4812, as described above) as the female parent and the haploid inducer line pollen-donor as the male parent, as this ensures that the normal A cytotype is transmitted to all progeny. This cytotype will confer an advantage in transformability. It is also preferable to start from a high-performing (>15% haploid induction rate) inducer and a highly transformable variety (transformation frequency >15%). It may also be helpful to select for high pollen load or tassel weight.

In this Example, a haploid inducer BC1 material “RWKS/Z21S//RWKS” (BC1 means backcross1 generation=75% RWKS and 25% Z21S) was crossed to two transformable varieties. The haploid inducer has a 15-18% haploid induction rate (very good) and a 0% transformation rate. This inducer was used as a male pollen donor and crossed onto the ears from two commercially important transformable maize lines: i) SYN-INBB23 (a non-stiff stalk line), which has a 5% transformation frequency, high pollen load and 0% haploid induction rate; and ii) SYN-INBC34 (a stiff stalk line), which has a 50% transformation frequency, high pollen load and 0% haploid induction rate. These crosses were made at RTP, North Carolina research station in the spring of 2018. The SYN-INBB23 and SYN-INBC34 lines were both identified as “Normal A” cytotype, whereas the RWKS/Z21S//RWKS line has a “Normal B” cytotype. In the assays shown in Table 9, SM2918 and SM4813 distinguish CMS cytoplasm (A/A) from normal A or B cytoplasm (C/C); all three of the lines used to start the breeding process have normal cytoplasm (C/C for markers SM2918 and SM4813). SM2914 and SM4812 distinguish Normal B cytoplasm (A/A and D/D respectively) from Normal A cytoplasm (C/C and I/I respectively). Normal A cytoplasm is associated in maize with an ability to be transformed and regenerate transgenic plants (see, for example, WO 2020/205334 by Skibbe et al.), whereas Normal B cytoplasm is more recalcitrant to transformation.

TABLE 9
Assays used to detect the cytoplasm type of maize lines

| | | | | | Cytotype |
|---|---|---|---|---|---|
| Marker | SM2918 | SM4813 | SM2914 | SM4812 | call |
| Chromosome | Mitochondrial genome | | | | |
| PanDa Assay UID | 484085 | 999991175 | 507573 | 1136316 | |
| RWKS/Z21/RWKS | C/C | C/C | A/A | D/D | Normal B |
| SYN-INBB23 | C/C | C/C | C/C | I/I | Normal A |
| SYN-INBC34 | C/C | C/C | C/C | I/I | Normal A |

“I” in Assay SM4812: insertion allele; “D” in Assay SM4812: deletion allele.

RWKS/Z21S//RWKS is identified as the haploid inducer material. Importantly, this haploid inducer has the native matrilineal mutation (matl; 4 bp insertion). The mutation in MATRILINEAL (a.k.a. NOT LIKE DAD and PLA1; maize B73 gene ID GRMZM2G471240 on Chromosome 1) has been described in, e.g., U.S. Pat. No. 10,448,588 by Kelliher et al. and Kelliher, et al., Nature, 2017. The mutation in MATRILINEAL confers a maternal haploid induction rate (HIR) of 1% to 7% alone (i.e. without the qhir8 HI allele, see below). HIR refers to the proportion of offspring of the cross that are haploid; the other 93 to 99% are diploid. This rate can be affected by environmental conditions as well as genetic backgrounds of both the inducer line and the female line that is crossed to. The haploid induction rate results shown below are typical for haploid inducer (e.g. RWKS/Z21S//RWKS) and non-inducer (e.g. SYN-INBB23 or SYN-INBC34) lines. RWKS/Z21S//RWKS has a higher induction rate because it has HI alleles at other QTLs (e.g., qhir8, see below) or genes which confer a higher HIR in combination with matl. The 4 bp insertion matl allele can be detected using site-specific SNP markers (SM7246 and SM7252) or allele-specific TaqMan markers (Assays 2826 & 2827), as described below and in Table 10. Assay 2826 detects the wild-type allele; assay 2827 detects the 4 bp insertion, a mutant allele that triggers haploid induction. Note, the “I” genotype in Assay SM7252 refers to the four base pair insertion allele in the matrilineal gene in the haploid inducer line (which causes a knockout of the gene) and the “D” genotype refers to the allele where there is no insertion. The “D” allele is the wild-type version of the gene where there is a functional protein product.

TABLE 10
Assays and markers used to detect the inducer and
non-inducer alleles of the MATRILINEAL gene.

| | | | | | Avg. |
|---|---|---|---|---|---|
| | | | | | Haploid |
| | | | | | Induction |
| Marker | SM7246 | SM7252 | Assay 2826 | Assay 2827 | Rate (HIR) |
| PanDa or TaqMan | 4057030 | 4057033 | TQ1838 | TQ1839 | |
| Assay ID | | | | | |
| Location (B73v5) | 69430544 | 69430644 | 69430644 | 69430644 | |
| RWKS/Z21S/RWKS | G/G | I/I | 0 copy | 2 copy | 12-20% |
| SYN-INBB23 | C/C | D/D | 2 copy | 0 copy | 0.1% |
| SYN-INBC34 | C/C | D/D | 2 copy | 0 copy | 0.1% |

In addition, RWKS/Z21S//RWKS has a haploid induction enhancer allele (HI allele) at a locus referred to in prior work as qhir8, which is located on Chromosome 9. This allele enhances the haploid induction rate. Combined with the matl mutant allele, the haploid induction rate is boosted by this QTL allele to about 10 to 20%, depending on a range of other factors such as other QTLs/genes, environmental conditions, female germplasm genetic group, and potentially other factors. The markers in Table 11 can be used to distinguish lines that have the qhir8 HI allele from those that do not. The position of qhir8 has been mapped between markers SM4849 and SM0956BQ. Fine mapping and genome editing has indicated the gene responsible for the qhir8 HI allele is GRMZM2G465053 (B73 v4) or Zm00001d044822 (B73v5), known as DUF679 domain membrane protein 7 (DMP for short) and it is located in the B73v5 genome between locations 3,919,235 and 3,919,852. The assay SM8133 is located in the DMP gene.

TABLE 11
Assays used to genotype the qhir8 region.

| | Marker | | | | | | |
|---|---|---|---|---|---|---|---|
| | SM4849 | SM8047 | SM8133 | SM8029 | SM4257 | SM0956BQ | Avg. |
| | PanDa Assay UID | | | | | | Haploid |
| | 1136395 | 4519582 | 4519723 | 4519600 | 1004889 | 482610 | Induction |
| | Location (B73v5) | | | | | | Rate |
| | 3444422 | 3880187 | 3920009 | 4580317 | 7248352 | 11360090 | (HIR) |
| RWKS/Z21/RWKS | A/A | T/T | C/C | A/A | G/G | G/G | 12-20% |
| SYN- | G/G | | | | G/G | A/A | 0.1% |
| INBB23 | | | | | | | |
| SYN- | G/G | A/A | G/G | G/G | C/C | A/A | 0.1% |
| INBC34 | | | | | | | |

Blank spaces indicate that the genotype is unknown or cannot be determined using those assays.

During a matl-based haploid induction cross, the vast majority of the resulting embryos are diploids (usually between 65 and 99%) and around 1 to 35% are haploids (averaging perhaps 15-20% for “good” haploid inducers). It is important for doubled haploid breeding pipelines to have a genetically-controlled visual trait to identify those embryos that lack the inducer chromosome set or DNA (haploids) and sort them from those that have the inducer chromosome set or DNA (diploids). In maize, most doubled haploid breeding pipelines utilize inducer lines that carry an allele of the R1 gene that confers a dominant anthocyanin trait which will express a purple or red color in the both the embryo and endosperm of the seed. There are at least two options: R-navajo (R-nj), an allele associated with very strong expression in the aleurone layer (outermost later of the endosperm) and weaker expression in the embryo, and R1-scutellum (R1-SCM2), an allele associated with strong expression in the immature embryo and weaker expression in the endosperm aleurone. Both alleles are associated with the R1 locus, which is on chromosome 10, around position ˜139 Mb to ˜140 Mb in version 5 of the maize B73 inbred genome (B73v5). The inducer used for breeding in this Example, RWKS/Z21S//RWKS, has R1-SCM2. The SYN-INBB23 and SYN-INBC34 plant lines, like most maize elite germplasm, do not have this allele or the R-nj allele at the R1 locus (these lines are wild-type for R1 and do not have any color induction in the seed or kernel). Critically, the R-nj and R1-SCM2 color-inducing alleles are dominant to wild-type. Maize germplasm can be assayed using the following 3 linked markers (shown in Table 12) to distinguish the R1-SCM2 (RWKS/Z21S//RWKS) allele from the wild-type SYN-INBB23 and SYN-INBC34 alleles. For SM0953BQ, SM6568, and SM0954BQ, the R1-SCM2 genotypes are A/A, T/T, and C/C, whereas the wild-type genotypes are G/G, A/A, and A/A, respectively.

Additionally, there is a color inhibitor locus in the SYN-INBC34 germplasm on Chromosome 9, between position 8 Mb and 10 Mb. A color inhibitor allele at this locus prevents the accumulation of embryo pigment triggered by R1-SCM2. While the mechanism is not clear, if this allele is present in the context of R1-SCM2, the embryo color might not be as strong as normal. During breeding of new inducers with SYN-INBB23, the color-inducing R1-SCM2 allele from the RWKS/Z21S//RWKS parent was selected for; during breeding of new inducers with SYN-INBC34, the same allele was selected for in addition to the wild-type allele (i.e., not the color inhibitor allele) for the color inhibitor on Chromosome 9 (using the assays shown in Table 12), so the resulting inducers would have a strong color potential.

TABLE 12
Assays used to genotype the color marker and color inhibitor alleles.

| | Marker | | | | | | | |
|---|---|---|---|---|---|---|---|---|
| | SM0954BQ | SM0954HQ | SM6568 | SM0953BQ | SM6604 | SM8040 | SM8091 | |
| | Chrom. | | | | | | | |
| | 10 | | | | | 9 | | |
| | Trait/QTL/Gene | | | | | | | |
| | R1-SCM2 dominant color inducer | | | | | Color inhibitor | | |
| | Location (B73v5) | | | | | | | |
| | 139300360 | 139300732 | 139789170 | 140146379 | 139789786 | 8120045 | 10040065 | |
| | PanDa Assay UID | | | | | | | Embryo |
| | 537327 | 545372 | 3775499 | 535601 | 3775416 | 4519585 | 4519705 | Color |
| RWKS/Z21//RWKS | C/C | G/G | T/T | A/A | G/G | G/G | C/C | Purple |
| SYN-INBB23 | C/C | A/A | T/T | A/A | A/A | | | Cream |
| SYN-INBC34 | A/A | | A/A | G/G | | A/A | A/A | Cream |

Blank spaces indicate that the genotype is unknown or cannot be determined using those assays.

### Breeding HI-NA Lines

The first step of breeding a new transformable haploid inducer is critical: a transformable line (e.g. SYN-INBB23 or SYN-INBC34) is used as the female in a cross with a haploid inducer line (e.g. RWKS/Z21S//RWKS). Such a cross will automatically confer Normal A-cytoplasm to the F1 offspring due to maternal cytoplasmic inheritance—that is, the female egg cell donates its mitochondria (and thus the mitochondrial genome) to its progeny, whereas the male germ cell (sperm cell, found in pollen grains) does not transfer mitochondria to progeny. About 20 F1 plants were grown up and self-pollinated, and a few other F1 plants were backcrossed to RWKS/Z21S/RWKS to generate about 7000 F2 or “BC1” (backcross generation 1) progeny in total. These progeny seed automatically inherit the Normal A cytoplasm as well. The SYN-INBB23 and SYN-INBC34 seed were sorted for the R1-SCM2 trait by selecting seeds with a purple color. Yellow seeds were discarded. Yellow seeds made up about ¼ of the total seed (¾ were purple), owing to Mendelian segregation of the dominant R1-SCM2 allele. As described below, seedlings were germinated and leaf punches were taken from the ˜5200 purple-seeded BC1 or F2 plants and genotyped for the haploid inducer markers described above.

The SYN-INBB23×RWKS/Z21S/RWKS F2 and BC1 generation was grown in the RTP, North Carolina greenhouse in the winter of 2018/2019. All germinated plants (5219 in total) were sampled—four leaf punches were obtained from seedling leaf. DNA was extracted and TaqMan was run on the assays as outlined above. Specifically, real-time PCR was set up in multiplexed TaqMan reactions to simultaneously amplify the target gene and an endogenous control gene. For each sample, the assay was setup by combining the extracted genomic DNA sample with TaqMan PCR master mix (containing Jumpstart Taq ReadyMix (Sigma) supplemented with primers and probes). Real-time PCR was carried out in Real-time PCR machines, using the following parameters: 95° C. for 5 minutes, 40 cycles of 95° C. for 5 seconds and 60° C. for 30 seconds. Post-run data analysis was performed according to the manufacturer's instructions. For additional TaqMan procedural details, see, e.g., U.S. Patent Application Publication No. 2011/0300544 (filed Dec. 7, 2009), incorporated herein by reference in its entirety.

After scoring the genotyping calls, out of 5000+ plants, 117 that genotyped as having favorable haploid inducer genotypic combinations were selected (see Table 13 for summary of plants that were selected and not selected, along with their genotypes), transplanted to large pots and self-pollinated to make seed for the next generation, though a few were haploids and could not be self-pollinated due to sterility. Many individuals were found to be fixed homozygous for all haploid inducer loci, though due to segregation distortion against matl and the qhir8 HI allele, additional plants that were heterozygous for R1-SCM2 or the qhir8 HI allele were kept in order to maintain greater genetic diversity among the F3/BC1F2 families. Progeny coming from those plants are segregating so they needed to be genotyped as F3/BC1F2 generation before HIR phenotyping (testcrossing), as described below. The color inhibitor, which was not known to affect color induction in the SYN-INBB23 background, was consistent fixed for the haploid inducer allele (i.e., the wild-type allele) in this set of 117 plants to ensure color induction. Selected F3 or BC1F2 ears were sent to Janesville, Wisconsin for the summer of 2019 (described below).

TABLE 13
Selection of SYN-INBB23 F2 and BC1 plants for next generation.

| | Chrom. | | | | | | |
|---|---|---|---|---|---|---|---|
| | 1 | 1 | 9 | 9 | 10 | 10 | |
| | Assay | | | | | | |
| | SM7252 | SM7246 | SM4849 | SM0956BQ | SM0954HQ | SM6604 | |
| | PanDa Assay | | | | | | |
| | 4057030 | 4057030 | 1136395 | 482610 | 545372 | 3775416 | Number |
| | Gene / QTL | | | | | | Of |
| | matl | matl | qhir8 | qhir8 | R1-SCM2 | R1-SCM2 | Plants |
| Fixed for all | II | GG | AA | GG | GG | GG | 30 |
| Fixed for all | II | GG | AA | GG | GG | GG | 22 |
| backcrossed | | | | | | | |
| R1-SCM2 het | II | GG | AA | GG | AG | AG | 38 |
| R1-SCM2 het | II | GG | AA | GG | AG | AG | 12 |
| backcrossed | | | | | | | |
| qhir8 het | II | GG | AG | GG | GG | GG | 15 |

| | | |
|---|---|---|
| other | Mixed Genotypes (discarded) | 5002 |

“II” in assay SM7252 refers to homozygosity for the mutant four base pair insertion mutant matrilineal allele that is responsible for haploid induction.

In the SYN-INBC34 F2 population, 194 individuals were identified to have favorable haploid inducer genotypic combinations (see Table 14 for summary of plants that were selected and not selected, along with their genotypes). These were selected and self-pollinated to make seed for the next generation, though some were haploids and could not be self-pollinated due to sterility. Several were fixed (homozygous) for all the haploid inducer loci. Due to segregation distortion against matl and the qhir8 HI allele, other plants found to be heterozygous for either R1-SCM2, matl or the qhir8 HI allele plus the color inhibitor allele near qhir8 on Chromosome 9 were kept in order to maintain greater genetic diversity among F3 families. Progeny coming from those plants were segregating, so they needed to be genotyped in the F3 generation before any HIR phenotyping (testcrossing), as is shown below. In the SYN-INBC34×RWKS/Z21S//RWKS F2, plants were also selected for heterozygosity or homozygosity of the RWKS/Z21S/RWKS allele for the color-inhibitor gene on Chromosome 9, to avoid bringing the SYN-INBC34 allele, which acts as an inhibitor of color accumulation. This SYN-INBC34×RWKS/Z21S//RWKS F2 population was grown in Janesville, Wisconsin during the summer of 2019. Selected F3 ears (from self-pollinated F2s) were then sent to Graneros, Chile for phenotyping as described below.

TABLE 14
Selection of SYN-INBC34 F2 plants for next generation.

| | Chrom. | | | | | | | |
|---|---|---|---|---|---|---|---|---|
| | 9 | 9 | 9 | 10 | 10 | 1 | 1 | |
| | Assay ID | | | | | | | |
| | 1136395 | 1004889 | 482610 | 537327 | 535601 | 4057033 | 4057030 | Number |
| Status | SM4849 | SM4257 | SM0956BQ | SM0954BQ | SM0953BQ | SM7246 | SM7252 | of plants |
| fixed | AA | GG | GG | CC | AA | GG | II | 30 |
| matl het | AA | GG | GG | CC | AA | CG | DI | 74 |
| qhir8/R1 | AG | CG | AG | CC | AA | GG | II | 54 |
| inhibitor | | | | | | | | |
| heterozygous | | | | | | | | |
| R1-SCM2 | AA | GG | GG | AC | AG | GG | II | 36 |
| het | | | | | | | | |

| | | |
|---|---|---|
| other | Mixed Genotypes (did not cross) | 3057 |

For the next SYN-INBB23×RWKS/Z21S//RWKS generation, 520 rows were planted at the Janesville, Wisconsin breeding station in the summer of 2019, comprising 95 F3 and 35 BC1F2 subfamilies, planted in quadruplicate, plus controls. For each row, one or more female tester rows were planted alongside. For SYN-INBC34×RWKS/Z21S//RWKS, 699 rows representing about 187 F3 subfamilies (planted in duplicate or quadruplicate) were planted at the Graneros, Chile breeding station alongside female tester rows. For most subfamilies, leaf samples were obtained for genotyping for the haploid inducer loci qhir8 and/or R1-SCM2 (and, for SYN-INBC34, for the color inhibitor locus). Selected individuals that were homozygous for the RWKS/Z21S//RWKS alleles were nominated to be testcrossed as males onto several ears from the female testers. In total, 777 individuals plus several controls were testcrossed from the SYN-INBB23 F3 generation. Similarly, 813 individuals, including controls, were testcrossed from the SYN-INBC34 F3 generation. The field team selected away from negative phenotypes such as low pollen production or large anthesis-silking interval. The haploid induction rate was determined by harvesting testcrossed ears at about 15 to 20 days after pollination, and then isolating the embryos from the kernels onto a petri dish containing embryo rescue media (4.43 grams of Murashige and Skoog basal media with vitamins, 30 grams of sucrose, and 70 milligrams of salicylic acid). The petri dishes were exposed to light (see, e.g., as described in WO2015/104358). The number of purple (diploid) and cream-colored (haploid) embryos were counted from each ear, to find the frequency of haploids, known as the haploid induction rate (HIR), and the total embryos per ear. The plants that were used for testcrossing were also self-pollinated; seed was collected from self-pollinated ears from this select set of F3 or BC1F2 plants to generate F4 or BC1F3 seed. In Table 15 (SYN-INBC34) and Table 16 (SYN-INBB23), the haploid induction rate, marker genotypes, and total embryos (divided into haploid and diploid embryos) from representative plots are shown, as well as some medium and low performing lines without matl and the qhir8 HI allele fixed. Table 15 shows F3 SYN-INBC34×RWKS/Z21S//RWKS lines fixed for all HI alleles, as well as lines that were homozygous wild-type or heterozygous for the qhir8 HI allele and/or matl. All lines were fixed for the color inhibitor allele and R1-SCM2 gene which gives the purple color embryo in diploids. The lines fixed for all inducer alleles ranged from 10-19% HIR, similar to the control (RWKS/Z21S//RWKS), whereas other lines had lower haploid induction rates. Table 16 shows F3 SYN-INBB23×RWKS/Z21S//RWKS lines fixed for all HI alleles. All lines were fixed for the R1-SCM2 gene which gives the purple color embryo in diploids.

From the F3 generation in SYN-INBB23×RWKS/Z21S//RWKS, 63 individual plants deriving from 47 different F2-derived families were identified that were fixed for the haploid inducer alleles for matl, qhir8 and R1-SCM2 and had at least a 10% haploid induction rate and at least 70 kernels per ear seed set. Those self-pollinated ears from these individuals that had at least 20 kernels were harvested and forwarded to the next generation and used to plant one or more male rows for additional testcrossing (F4 generation test crossing to determine haploid induction rate), and additionally in most cases some seed was forwarded for F4-generation transformation rate testing. Likewise, the data from the F3 generation in the SYN-INBC34×RWKS/Z21S//RWKS population was used to identify 71 individuals deriving from 41 F3 families that were fixed for the inducer genes and had a >10% haploid induction rate and high seed set, and where there were at least 20 kernels per self-pollinated ear. These were forwarded to the F4 generation.

TABLE 15
Haploid induction rate (HIR) and markers for representative F3 SYN-INBC34 x RWKS/Z21S//RWKS lines.

| | | matl | | qhir8 | | Color inhibitor | | R1-SCM2 |
|---|---|---|---|---|---|---|---|---|
| Line | Gen/Type | SM7246 | SM7252 | SM4849 | SM4257 | SM4263 | SM0956BQ | SM0954BQ |
| RWKS/Z21S//RWKS | Control/fixed all | GG | II | AA | GG | AA | GG | CC |
| 19BD915888 | F3/fixed all | GG | II | AA | GG | AA | GG | CC |
| 19BD915935 | F3/fixed all | GG | II | AA | GG | AA | GG | CC |
| 19BD915954 | F3/fixed all | GG | II | AA | GG | AA | GG | CC |
| 19BD915988 | F3/fixed all | GG | II | AA | GG | AA | GG | CC |
| 19BD915994 | F3/fixed all | GG | II | AA | GG | AA | GG | CC |
| 19BD915893 | F3/−qhir8 | GG | II | GG | CC | AA | GG | CC |
| 19BD915901 | F3/−qhir8 | GG | II | GG | CC | AA | GG | CC |
| 19BD915913 | F3/−qhir8 | GG | II | GG | CC | AA | GG | CC |
| 19BD915981 | F3/qhir8 het | GG | II | AG | GC | AA | GG | CC |
| 19BD915962 | F3/qhir8 het | GG | II | AG | GC | AA | GG | CC |
| 19BD916054 | F3/−matl | CC | DD | AA | GG | AA | GG | CC |
| 19BD915914 | F3/−matl | CC | DD | AA | GG | AA | GG | CC |
| 19BD915920 | F3/matl het | GC | DI | AA | GG | AA | GG | CC |
| 19BD915906 | F3/matl het | GC | DI | AA | GG | AA | GG | CC |
| Average all | F3/fixed all | GG | II | AA | GG | AA | GG | CC |
| (−outliers) | | | | | | | | |

| | | R1-SCM2 | | Embryo total | | | Embryo |
|---|---|---|---|---|---|---|---|
| Line | Gen/Type | SM0953BQ | Reps | Haploids | Diploids | HIR | Color |
| RWKS/Z21S//RWKS | Control/fixed all | AA | 22 | 1067 | 6488 | 16.4% | purple |
| 19BD915888 | F3/fixed all | AA | 4 | 191 | 999 | 19.1% | purple |
| 19BD915935 | F3/fixed all | AA | 4 | 409 | 2956 | 12.2% | purple |
| 19BD915954 | F3/fixed all | AA | 4 | 483 | 2176 | 18.2% | purple |
| 19BD915988 | F3/fixed all | AA | 4 | 292 | 1642 | 15.1% | purple |
| 19BD915994 | F3/fixed all | AA | 4 | 288 | 2393 | 10.7% | purple |
| 19BD915893 | F3/−qhir8 | AA | 3 | 146 | 2276 | 6.0% | purple |
| 19BD915901 | F3/−qhir8 | AA | 4 | 130 | 1756 | 6.9% | purple |
| 19BD915913 | F3/−qhir8 | AA | 2 | 15 | 535 | 2.7% | purple |
| 19BD915981 | F3/qhir8 het | AA | 2 | 21 | 296 | 6.6% | purple |
| 19BD915962 | F3/qhir8 het | AA | 1 | 23 | 216 | 9.6% | purple |
| 19BD916054 | F3/−matl | AA | 1 | 6 | 1159 | 0.5% | purple |
| 19BD915914 | F3/−matl | AA | 1 | 20 | 1709 | 2.9% | purple |
| 19BD915920 | F3/matl het | AA | 1 | 12 | 465 | 2.5% | purple |
| 19BD915906 | F3/matl het | AA | 1 | 37 | 1048 | 3.4% | purple |
| Average all | F3/fixed all | AA | 385 | 24,241 | 185,456 | 11.6% | purple |
| (−outliers) | | | | | | | |

“Reps” refers to the replicate number; each replicate consisted of the pollen from one male plant testcrossed onto at least four elite hybrid tester ears each. The final row has the totals for all plant lines fixed for the inducer QTL allele in this generation.

TABLE 16
Haploid induction rate (HIR) and markers for representative
F3 SYN-INBB23 x RWKS/Z21S//RWKS lines.

| | | Matl | | qhir8 | | R1-SCM2 | |
|---|---|---|---|---|---|---|---|
| Line | Type | SM7246 | SM7252 | SM4849 | SM4257 | SM0954BQ | SM0953BQ |
| RWKS/Z21S//RWKS | Control | GG | II | AA | GG | CC | AA |
| 19SBI110630 | F3 | GG | II | AA | GG | CC | AA |
| 19SBI110664 | F3 | GG | II | AA | GG | CC | AA |
| 19SBI110704 | F3 | GG | II | AA | GG | CC | AA |
| 19SBI110705 | F3 | GG | II | AA | GG | CC | AA |
| 19SBI110757 | F3 | GG | II | AA | GG | CC | AA |
| 19SBI110711 | BC1F2 | GG | II | AA | GG | CC | AA |
| 19SBI110715 | BC1F2 | GG | II | AA | GG | CC | AA |
| 19SBI110722 | BC1F2 | GG | II | AA | GG | CC | AA |
| Average of | F3 | GG | II | AA | GG | CC | AA |
| all F3 fixed | fixed | | | | | | |

| | | | Embryo total | | | Embryo |
|---|---|---|---|---|---|---|
| Line | Type | Reps | Haploids | Diploids | HIR | Color |
| RWKS/Z21S//RWKS | Control | 10 | 1365 | 7514 | 15.4% | purple |
| 19SBI110630 | F3 | 2 | 141 | 888 | 13.7% | purple |
| 19SBI110664 | F3 | 3 | 327 | 1372 | 19.2% | purple |
| 19SBI110704 | F3 | 2 | 138 | 584 | 19.1% | purple |
| 19SBI110705 | F3 | 2 | 110 | 849 | 11.5% | purple |
| 19SBI110757 | F3 | 3 | 218 | 968 | 18.4% | purple |
| 19SBI110711 | BC1F2 | 2 | 227 | 1453 | 13.5% | purple |
| 19SBI110715 | BC1F2 | 2 | 194 | 999 | 16.3% | purple |
| 19SBI110722 | BC1F2 | 2 | 138 | 601 | 18.7% | purple |
| Average of | F3 | 87 | 4902 | 30,373 | 13.9% | purple |
| all F3 fixed | fixed | | | | | |

“Reps” refers to the replicate number; each replicate consisted of the pollen from one male plant testcrossed onto at least four elite hybrid tester ears each. The final row has the totals for all of the F3 plant lines fixed for the inducer QTL in this generation (minus outliers, and lines with low seed set due to poor nicking or asynchronous flowering of the tester line silks and the inducer pollen).

For the SYN-INBB23×RWKS/Z21S//RWKS F4 generation (140 male rows from 81 different plant lines based on data from F4 HIR phenotyping using one or, usually, two rows×2 male testers each in Graneros, Chile 2019/2020 season), genotyping was not done as all lines were now fully fixed for the haploid induction rate genes/loci. Haploid induction rate phenotyping was performed in the Arica, Chile breeding and doubled haploid facility, and transformation testing was performed on a subset of ˜60 of the same F4 plant materials at the RTP, North Carolina research center. For transformation rate testing, the binary vector #12672 was delivered to embryos pooled from between 3 and 10 self-pollinated F4 ears via Agrobacterium-mediated transformation using the strain LBA4404 (pAL4404, pVGW7). Detailed information about the pAL4404 and pVGW7 helper plasmid and the virulence region is described by the following references: Teruyuki Imayama, T. et al., Japan Patent Appl. No. 20160083737, JAPAN TOBACCO INC., JAPAN, 2016; Ishida, Y., High efficiency transformation of maize (Zea mays L.) mediated by Agrobacterium tumefaciens. Nat. Biotechnol. 14, 745-750 (1996); and Negrotto, D., et al., The use of phosphomannose-isomerase as a selectable marker to recover transgenic maize plants (Zea mays L.) via Agrobacterium transformation. Plant Cell Rep. 19, 798-803 (2000). The Agrobacterium strain containing the binary vectors and test constructs was prepared as described by Negrotto et al. (2000) cited above. For maize transformation, immature embryos from greenhouse grown maize inbred line NP2222 were used as explants according to Heng Zhong, et al., Advances in Agrobacterium-mediated Maize Transformation. Methods Mol Biol 1676, 41-59 (2018). Immature embryo isolation, Agrobacterium inoculation and co-cultivation of Agrobacterium with the immature embryos were performed as described by Zhong et al. as cited above. Transformed tissues and putative transgenic events were generated on media using mannose selection as described earlier (Negrotto et al. (2000)). Phenotyping results for F4 plant lines (plant materials) are summarized in Table 17.

TABLE 17
Haploid induction rate (HIR) and transformation
frequency (TF) of F4 plant materials from the
SYN-INBB23 × RWKS/Z21S//RWKS populations.

| Plant | | | | | Embryo |
|---|---|---|---|---|---|
| Material Id | # Explants | Events | TF | HIR | total |
| 19BD915005 | 184 | 0 | | 15.8% | 599 |
| 19BD915016 | 353 | 0 | | 11.0% | 615 |
| 19BD915019 | 199 | 5 | 2.5% | 7.9% | 783 |
| 19BD915044 | 119 | 0 | | 14.1% | 628 |
| 19BD915057 | 308 | 0 | | 9.9% | 835 |
| 19BD915066 | 146 | 0 | | 18.2% | 504 |
| 19BD915072 | 216 | 0 | | 11.9% | 573 |
| 19BD915086 | 140 | 0 | | 26.6% | 356 |
| 19BD915121 | 77 | 0 | | 16% | 761 |
| 19BD915147 | 184 | 19 | 10.3% | 15.2% | 552 |
| 19BD915158 | 80 | 9 | 11.3% | 12.2% | 156 |
| 19BD915186 | 109 | 0 | | 14.8% | 637 |
| 19BD915207 | 95 | 0 | | 20.8% | 859 |
| 19BD915229 | 186 | 1 | 0.5% | 13.0% | 604 |
| 19BD915245 | 296 | 7 | 2.4% | 14.6% | 840 |
| 19BD915243 | 40 | 0 | | 20.0% | 656 |
| 19BD915248 | 300 | 0 | | 11.9% | 489 |
| 19BD915263 | 120 | 0 | | 23.0% | 336 |
| 19BD915300 | 99 | 0 | | 15.1% | 329 |
| 19BD915311 | 96 | 0 | | 10.1% | 603 |
| 19BD915360 | 134 | 4 | 3.0% | 10.1% | 485 |
| 19BD915363 | 152 | 0 | | 10.1% | 773 |
| 19BD915409 | 328 | 0 | | 27.8% | 295 |
| 19BD915411 | 393 | 0 | | 12.3% | 607 |
| 19BD915416 | 82 | 0 | | 9.7% | 647 |
| 19BD915418 | 143 | 1 | 0.7% | 16.8% | 540 |
| 19BD915436 | 183 | 6 | 3.3% | 9.3% | 335 |
| 19BD915452 | 194 | 0 | | 8.7% | 883 |
| 19BD915465 | 270 | 0 | | 12.2% | 181 |
| 19BD915489 | 150 | 0 | | 13.3% | 358 |
| 19BD915561 | 237 | 0 | | 7.5% | 604 |
| 19BD915614 | 329 | 0 | | 21.5% | 771 |
| 19BD915690 | 150 | 0 | | 17.2% | 313 |
| 19BD915840 | 89 | 0 | | 15.4% | 622 |
| 19BD915847 | 245 | 0 | | 20.8% | 800 |
| 19BD915875 | 309 | 31 | 10.0% | 15.0% | 498 |
| 19BD915880 | 82 | 0 | | 9.3% | 215 |

Most events were not transformable with the construct and protocol tested, but many events had strong haploid induction rates. This makes sense because the haploid induction rate genes were selected in the F2 and F3 generations, and the phenotype was selected in the F3, whereas there has been no selection on transformability (and there was only a ˜5% transformation frequency to start with in SYN-INBB23 and 0% in RWKS/Z21S//RWKS). Note, the haploid induction rate is based on several testcrossed ears across two tester lines. The embryo total (i.e., seed set) was strongly affected by the nick and the number of ears that were pollinated. Embryo total was not used as a selection metric; it is merely meant to show the number of embryos on which the haploid induction rate is based. Three plant materials were identified with a 10%+ transformation rate: 19BD915147, 19BD915875 and 19BD915158. The first two also had a promising >15% HIR.

Further HIR testing was used to narrow in on the best inducers that were also transformable. In the summer of 2020 at the Janesville, Wisconsin breeding station, 41 SYN-INBB23×RWKS/Z21S//RWKS F5 generation male rows, deriving from 10 F3 generation families and several F4 subfamilies, were planted alongside three female tester rows for testcrosses to evaluate the haploid induction rate. Seed from some of these lines were also sent to the North Carolina facility to be retested for transformation frequency. The results of a select set of F4 and F5 generation trials are shown in Table 18. These five lines are high performing haploid inducer lines in the F5 generation; there is some evidence of transformability in the first three lines (either from F4 or F5 transformation rate testing). The lines shown are those chosen for forwarding to HI-Edit experiments in the F6 generation, as described below.

TABLE 18
Haploid induction rate (HIR) and transformation frequency (TF) testing
in the SYN-INBB23 x RWKS/Z21S//RWKS F4 and F5 generation.

| F4 Plant | F4 TF | F4 | F5 Plant | F5 TF | F5 | | Haploids/ |
|---|---|---|---|---|---|---|---|
| Material ID | Rate | HIR | Material ID | rate | HIR | F5 Seed Set | ear (F5) |
| 19BD915147 | 10.3% | 15.2% | 19SN952821 | n/a | 12.7% | 146 | 19 |
| | | | 19SN952822 | n/a | 13.7% | 194 | 26 |
| 19BD915086 | 0% | 26.6% | 19SN952871 | 1.3% | 17.0% | 173 | 31 |
| 19BD915121 | 0% | 16% | 19SN952763 | n/a | 10.3% | 284 | 29 |
| 19BD915243 | 0% | 20.0% | 19SN953098 | n/a | 18.0% | 172 | 31 |

F6 generation seed was harvested from the self-pollinated ears from these plants and forwarded to the next generation for HI-Edit testing. See Example 2 for the CRISPR-Cas transformation and HI-Edit testing. In previous tests, using BBM increased the transformation frequency of the parent plant material (see Table 19).

TABLE 19
BBM-mediated transformation increases transformation
efficiency in parent plant material.

| | 12672 (Control) | 23967 (BBM) |
|---|---|---|
| Plant Material: | 45 events/400 | 99 events/400 |
| SYN-INBB23 | embryos | embryos |

For the SYN-INBC34×RWKS/Z21S//RWKS F4 generation, a haploid induction trial was conducted in the Janesville, Wisconsin breeding station in the summer of 2020. The 71 selected plant materials mentioned above were planted and testcrossed by three sets of female tester ears; at 15 to 20 days after pollination the ears were once again shipped to the RTP, NC site for HIR evaluation. At the same time, transformation rate of a subset of lines was determined at the RTP site using the same transformation process as described above, and F5 generation seed was obtained from a selected subset of self-pollinated plants and forwarded to the next generation. Results are shown in Table 20.

TABLE 20
Haploid induction rate (HIR) and transformation frequency (TF) of F4
plant materials from the SYN-INBC34 x RWKS/Z21S//RWKS populations.

| | F3 Plant | | | | Total # | | | |
|---|---|---|---|---|---|---|---|---|
| Plant | Material ID | | | | of Tester | Induction | Haploids/ | Average |
| Material ID | (family) | Embryos | Events | TF % | Ears | Rate | ear | Seed set |
| 19SN951894 | 19BD915935 | 206 | 0 | 0.0% | 12 | 15.7% | 35 | 221 |
| 19SN951905 | 19BD915954 | not tested | | | 11 | 15.3% | 21 | 135 |
| 19SN951915 | 19BD916026 | not tested | | | 11 | 14.5% | 18 | 129 |
| 19SN951924 | 19BD915945 | 228 | 0 | 0.0% | 12 | 21.7% | 40.6 | 188 |
| 19SN951926 | 19BD915950 | not tested | | | 11 | 13.3% | 31 | 232 |
| 19SN951929 | 19BD915954 | 173 | 0 | 0.0% | 12 | 17.7% | 28.7 | 162 |
| 19SN951933 | 19BD915973 | 56 | 0 | 0.0% | 8 | 10.2% | 21.1 | 207 |
| 19SN951950 | 19BD915935 | 187 | 0 | 0.0% | 12 | 13.8% | 24 | 173 |
| 19SN951958 | 19BD915950 | not tested | | | 12 | 13.2 | 38.5 | 293 |
| 19SN951981 | 19BD915945 | 332 | 2 | 0.6% | 12 | 15.9% | 26.5 | 167 |
| 19SN951986 | 19BD915954 | 125 | 0 | 0.0% | 10 | 15.9% | 28 | 177 |
| 19SN952015 | 19BD915942 | 13 | 0 | 0.0% | 9 | 17.4% | 20 | 114 |
| 19SN952019 | 19BD915955 | not tested | | | 11 | 17.5% | 37 | 194 |
| 19SN952030 | 19BD915988 | 345 | 2 | 0.6% | 12 | 9.0% | 18 | 194 |
| 19SN952034 | 19BD916005 | 133 | 0 | 0.0% | 11 | 11.3% | 20 | 177 |
| 19SN952043 | 19BD916033 | 361 | 5 | 1.4% | 12 | not tested | | |
| 19SN952047 | 19BD916050 | 210 | 1 | 0.5% | 12 | 12.3% | 25.4 | 207 |
| 19SN952059 | 19BD915953 | 80 | 0 | 0.0% | 11 | 14.5% | 30 | 205 |
| 19SN952060 | 19BD915957 | 87 | 0 | 0.0% | 11 | 6.4% | 18 | 281 |
| 19SN952067 | 19BD915987 | 215 | 0 | 0.0% | 9 | 18.2% | 25 | 141 |
| 19SN952072 | 19BD916020 | not tested | | | 11 | 18.0% | 36 | 200 |
| 19SN952080 | 19BD916043 | not tested | | | 6 | 6.0% | 14 | 234 |
| 19SN952091 | 19BD915891 | 174 | 0 | 0.0% | 9 | 13.5% | 27 | 200 |
| 19SN952123 | 19BD915978 | 166 | 0 | 0.0% | 11 | 16.2% | 28.5 | 179 |
| 19SN952139 | 19BD916024 | 259 | 0 | 0.0% | 7 | 15.5% | 27.7 | 176 |
| 19SN952153 | 19BD916055 | 320 | 0 | 0.0% | 11 | 13.7% | 23.4 | 171 |
| 19SN952161 | 19BD915967 | 316 | 24 | 8.1% | 9 | 11.4% | 15.3 | 134 |
| 19SN952183 | 19BD916075 | 273 | 24 | 9.0% | 9 | 11.9% | 26.8 | 225 |
| 19SN952188 | 19BD916051 | not tested | | | 11 | 19.0% | 30.3 | 156 |
| 19SN952189 | 19BD916051 | not tested | | | 11 | 11.4% | 13.6 | 119 |
| 19SN952195 | 19BD916017 | not tested | | | 9 | 15.2% | 20 | 134 |
| 19SN952196 | 19BD916022 | 265 | 148 | 58.5% | 10 | 13.3% | 27.2 | 204 |
| 19SN952199 | 19BD916035 | 188 | 31 | 16.5% | 12 | 11.0% | 20.2 | 182 |
| 19SN952200 | 19BD916035 | 146 | 3 | 2.1% | 11 | 14.5% | 21.5 | 149 |
| 19SN952262 | 19BD915987 | 228 | 0 | 0.0% | 12 | 9.0% | 20.2 | 224 |
| 19SN952285 | 19BD915902 | not tested | | | 12 | 12.3% | 20 | 163 |
| 19SN952311 | 19BD915983 | 94 | 0 | 0.0% | 12 | 11.0% | 24.8 | 225 |
| 19SN952331 | 19BD916028 | not tested | | | 12 | 13.0% | 18 | 146 |
| 19SN952346 | 19BD916055 | 320 | 4 | 1.3% | 12 | 7.6% | 20 | 268 |
| 19SN952347 | 19BD916055 | 320 | 2 | 0.6% | 12 | 7.0% | 16 | 227 |
| 19SN952348 | 19BD916055 | 231 | 1 | 0.4% | 10 | 10.0% | 17 | 177 |
| 19SN952358 | 19BD915967 | 159 | 2 | 1.3% | 12 | 9.9% | 16.2 | 165 |
| 19SN952361 | 19BD915907 | 166 | 2 | 1.2% | 4 | 5.0% | 12.5 | 250 |
| 19SN952402 | 19BD915942 | 68 | 0 | 0.0% | 11 | 15.1% | 25 | 170 |
| 19SN952406 | 19BD915952 | 40 | 0 | 0.0% | 12 | 12.9% | 22 | 181 |
| 19SN952408 | 19BD915956 | 259 | 0 | 0.0% | 12 | 6.5% | 15 | 226 |
| 19SN952424 | 19BD915995 | 178 | 0 | 0.0% | 12 | 12.6% | 28.6 | 246 |
| 19SN952426 | 19BD915995 | not tested | | | 11 | 14.0% | 24 | 174 |
| 19SN952429 | 19BD916009 | 23 | 5 | 21.7% | 12 | 13.0% | 20 | 149 |
| 19SN952441 | 19BD916050 | not tested | | | 12 | 10.4% | 26 | 254 |
| 19SN952454 | 19BD915949 | 293 | 40 | 13.7% | 12 | 15.2% | 33.1 | 217 |
| 19SN952456 | 19BD915953 | not tested | | | 8 | 16.1% | 30 | 195 |
| 19SN952551 | 19BD915967 | 76 | 0 | 0.0% | 12 | 10.5% | 24 | 221 |
| 19SN952582 | 19BD915888 | not tested | | | 12 | 9.5% | 19.7 | 207 |
| 19SN952610 | 19BD916050 | 300 | 0 | 0.0% | 12 | 11.8% | 32 | 278 |
| 19SN952627 | 19BD915984 | 13 | 0 | 0.0% | 12 | 15.4% | 34 | 223 |
| 19SN952646 | 19BD915966 | 111 | 3 | 2.7% | 12 | 11.1% | 27 | 248 |
| 19SN952670 | 19BD916024 | 159 | 3 | 0.0% | 11 | 10.2% | 23 | 216 |
| 19SN952688 | 19BD915967 | 64 | 0 | 0.0% | 12 | 7.0% | 10.4 | 149 |

Few lines exhibited a high transformation frequency in this experiment, but most lines had very high haploid induction rates (most over 10% and some over 15%). This outcome is consistent with the selection of haploid induction genes (markers) in the F2 and F3 generations and selection of high HTR phenotype in the F3 generation. Thus, this population was enriched for haploid induction rate genetics. In contrast, there had been no selection on transformability phenotypes or genes (markers) to this point, beyond the use of a Normal A cytotype maternal parent (SYN-IN4BC34) in the founding hybrid cross. The HTR is based on several testcrossed ears across two tester lines, and the total number of ears is provided. The seed set could be affected by the anthesis-silking interval (the synchronization of the inducer male pollen shedding window and the female tester ear silking date). In this experiment, the anthesis-silking interval was lower than in the F3 generation (i.e., there was a reduction in the time between pollen shedding and tester ear silking). Therefore, in addition to haploid induction rate and transformation rate, seed set is being used as a selection metric to identify the best lines for forwarding to the next round of evaluation (the F5 generation). The seed set average was found by dividing the total number of normal (non-aborted, endosperm-viable) kernels by the number of ears, combining both testers.

Two F4 lines displayed promising performance. First there is the line 19SN952196, which had a 58% transformation frequency (TF) (higher than any other known maize line transformation rate), a promising HIR above 13%, and good seed set. Secondly, there is the line 19SN952454, which had a 13.7% TF, HIR above 15%, and good seed set. These two lines averaged 27 and 33 haploids per ear, respectively. Photographs of the tester ears revealed that there was not a perfect synchronization between male and female—the ears may have been pollinated a little early, because the top ⅓ of the ear was not pollinated. It is therefore likely that the seed set and haploids per ear metrics could be even higher. Several individual plants from these two plant materials were self-pollinated to generate F5 seed lots for further evaluation, including haploid induction rate performance testing as well as transformation using a CRISPR-Cas construct to evaluate the HI-Editing rate across different maize varieties (HI-Edit spectrum tests). The F5 lines derived from 19SN952196 were transformed using a simple Agrobacterium-based process, as outlined above for the F4 generation, but this time using a CRISPR-Cas construct. The F5 lines from 19SN952454 were also transformed using a simple Agrobacterium-based process, as outlined above for the F4 generation, but this time using a CRISPR-Cas construct. Additionally, the F5 lines from 19SN952454 were transformed using a BBM-assisted transformation process, where a BBM construct and the CRISPR-Cas construct were co-transformed together to improve the transformation frequency of the CRISPR-Cas construct. See Example 2 for transformations.

Additionally, there were a few lines with strong haploid induction rate performance but without good transformation frequency (e.g. 19SN951924) or without transformation rate data (due to a lack of seed available for testing, e.g. 19SN951958, 19SN952019, and 19SN952072). These lines averaged between 35 and 40 haploids per ear. Several individuals from these four F4 plant materials were self-pollinated to generate F5 seed lots for further evaluation, including additional haploid induction rate performance testing as well as transformation using a CRISPR-Cas construct to evaluate the HI-Editing rate across different maize varieties (HI-Edit spectrum tests). The F5 lines derived from 19SN951924, 19SN951958, 19SN952019, and 19SN952072 were transformed via BBM-assisted transformation, where a BBM construct and the CRISPR-Cas construct were co-transformed together to improve the transformation frequency (see Example 2).

To identify genetic factors responsible for transformability in the SYN-INBC34 background, the parent plants used to generate the F4 lines studied above (in Table 20) were genotyped using 480 polymorphic SNP markers spread evenly across the maize genome. GWAS analysis of the lines in Table 20 that were transformation tested led to the identification of a QTL on chromosome 3, between markers SM3158 (genotype of SYN-INBC34 is GG, marker is at B73v5 position 14,742,407) and and SM4586 (genotype of SYN-INBC34 is GG, marker is at B73v5 position 70,562,070). The markers SM4787 (SYN-INBC34 genotype of GG), SM3814 (SYN-INBC34 genotype of CC), SM3362 (SYN-INBC34 genotype of GG), and SM0634AQ (SYN-INBC34 genotype of GG) are positioned in between SM3158 and SM4586 and may also work to identify the QTL. Comparing the set of lines with >5% transformation frequency to those with 0%, SM4787 has a GWAS log 10 value of 1.7 and a p-value <0.02. and SM4586 has a log 10 value of 0.57, and SM3362 has a log 10 value of 0.90. Comparing the set of lines with >5% TF to those with <5% TF, SM3814 has a GWAS log 10 value of 1.6, and SM4787 and SM3158 are 1.3. In Table 21 below, the genotypes of all of the TF-tested plant lines are shown. Seven of the top ten transformable lines with the highest transformation rate have the favorable SYN-INBC34 genotype (underlined). A large plurality of the <1% TF or non-transformable lines have the unfavorable (RWKS) alleles.

Based on this information, selection for plants having the SYN-INBC34 allele at this QTL (referred to herein as qCYTO-A_TF3.1) in the F2, F3, or any other generation may enrich the resulting lines for transformability (i.e., it may lead to higher transformation frequencies), in combination with Normal A cytotype or alone. This high transformation rate QTL allele has not been identified in prior work on maize transformation. Without being bound by any particular theory, it may be that the nuclear—cytoplasmic interaction or communication fostered by one or more loci in the Normal A cytotype (either in mitochondria or chloroplast), in combination with one or more loci in this QTL, combine to yield high transformation rates.

TABLE 21
Transformation rates of various lines (“plant materials”).

| | | | | SM3158 | SM4787 | SM3814 | SM3362 | SM0634AQ | SM4586 | |
|---|---|---|---|---|---|---|---|---|---|---|
| | | | | Chromosome | | | | | | |
| | | | | 3 | 3 | 3 | 3 | 3 | 3 | |
| | | | | Map position | | | | | | |
| | | | | 40.8 | 42.4 | 47.4 | 51.5 | 58.5 | 63.2 | |
| F4 Plant | | | | Physical position | | | | | | |
| Material ID | Calli | Events | TF | 14,742,407 | 19,210,942 | 28,710,708 | 32,750,671 | 58,719,312 | 70,562,070 | Genotype |
| SYN- | 1340 | 619 | 49.1% | G/G | G/G | C/C | G/G | G/G | G/G | Favorable |
| INBC34 | | | | | | | | | | |
| 19SN952196 | 265 | 148 | 58.5% | A/G | A/G | A/C | A/G | GG | G/G | Heterozygous |
| 19SN952429 | 23 | 5 | 21.7% | G/G | G/G | C/C | G/G | C/G | A/G | Favorable |
| 19SN952199 | 188 | 31 | 16.5% | A/G | A/G | A/C | A/G | C/G | A/G | Heterozygous |
| 19SN952454 | 293 | 40 | 13.7% | G/G | G/G | C/C | G/G | G/G | G/G | Favorable |
| 19SN952183 | 273 | 24 | 9.0% | G/G | G/G | C/C | G/G | G/G | G/G | Favorable |
| 19SN952161 | 316 | 24 | 8.1% | G/G | G/G | C/C | G/G | G/G | G/G | Favorable |
| 19SN952646 | 111 | 3 | 2.7% | A/G | A/G | A/C | A/G | C/G | A/G | Heterozygous |
| 19SN952200 | 146 | 3 | 2.1% | G/G | G/G | A/C | A/G | C/G | A/G | Favorable |
| 19SN952253 | 51 | 1 | 2% | G/G | G/G | C/C | G/G | G/G | G/G | Favorable |
| 19SN952043 | 361 | 5 | 1.4% | Z/Z | Z/Z | Z/Z | Z/Z | Z/Z | A/G | N/A |
| 19SN952358 | 159 | 2 | 1.3% | A/G | A/G | C/C | G/G | G/G | G/G | Recombinant |
| 19SN952346 | 320 | 4 | 1.3% | A/A | A/A | A/A | A/A | C/C | A/A | Unfavorable |
| 19SN952361 | 166 | 2 | 1.2% | A/A | A/A | A/A | A/A | C/G | A/G | Unfavorable |
| 19SN952030 | 345 | 2 | 0.6% | G/G | G/G | C/C | G/G | G/G | G/G | Favorable |
| 19SN952347 | 320 | 2 | 0.6% | A/A | A/A | A/A | G/G | G/G | G/G | Unfavorable |
| 19SN951981 | 332 | 2 | 0.6% | G/G | G/G | C/C | A/A | C/C | A/A | Favorable |
| 19SN952047 | 210 | 1 | 0.5% | A/A | A/A | A/A | A/A | C/C | A/A | Unfavorable |
| 19SN952348 | 231 | 1 | 0.4% | A/A | A/A | A/A | A/A | C/C | A/A | Unfavorable |
| 19SN952091 | 174 | 0 | 0.0% | A/A | A/A | A/A | A/A | C/C | A/A | Unfavorable |
| 19SN952015 | 13 | 0 | 0.0% | G/G | G/G | Z/Z | G/G | G/G | G/G | Recombinant |
| 19SN952402 | 68 | 0 | 0.0% | A/G | A/G | A/C | G/G | G/G | G/G | Heterozygous |
| 19SN952406 | 40 | 0 | 0.0% | A/G | A/G | A/C | A/G | C/G | A/G | Recombinant |
| 19SN952059 | 80 | 0 | 0.0% | A/G | A/G | A/C | A/G | C/G | A/G | Heterozygous |
| 19SN952408 | 259 | 0 | 0.0% | G/G | G/G | C/C | A/A | C/C | A/A | Favorable |
| 19SN952060 | 87 | 0 | 0.0% | A/A | A/A | A/A | A/G | C/G | A/G | Unfavorable |
| 19SN952025 | 285 | 0 | 0.0% | A/A | A/A | A/A | A/G | C/G | A/G | Unfavorable |
| 19SN952551 | 76 | 0 | 0.0% | A/A | A/A | C/C | A/G | C/G | G/G | Recombinant |
| 19SN952688 | 64 | 0 | 0.0% | A/A | A/A | C/C | A/G | C/G | A/G | Recombinant |
| 19SN952123 | 166 | 0 | 0.0% | G/G | G/G | C/C | G/G | G/G | G/G | Favorable |
| 19SN952311 | 94 | 0 | 0.0% | A/A | A/A | A/A | A/A | C/C | A/A | Unfavorable |
| 19SN952627 | 13 | 0 | 0.0% | A/A | A/A | A/A | A/A | C/C | A/A | Unfavorable |
| 19SN952067 | 215 | 0 | 0.0% | A/G | A/G | A/C | G/G | G/G | G/G | Heterozygous |
| 19SN952262 | 228 | 0 | 0.0% | A/A | A/A | A/A | G/G | G/G | G/G | Unfavorable |
| 19SN952424 | 178 | 0 | 0.0% | A/A | A/A | A/A | A/A | C/C | A/A | Unfavorable |
| 19SN952034 | 133 | 0 | 0.0% | A/G | A/G | Z/Z | G/G | G/G | G/G | Heterozygous |
| 19SN952139 | 259 | 0 | 0.0% | A/A | A/A | A/A | A/A | C/C | A/A | Unfavorable |
| 19SN952670 | 159 | 3 | 0.0% | A/G | Z/Z | A/C | A/A | C/C | A/A | Recombinant |
| 19SN952610 | 300 | 0 | 0.0% | A/G | A/G | A/C | A/G | C/G | A/G | Heterozygous |
| 19SN952153 | 320 | 0 | 0.0% | A/A | A/A | A/A | A/A | C/C | A/A | Unfavorable |
| 19SN951933 | 56 | 0 | 0.0% | G/G | G/G | A/A | A/A | C/C | A/A | Recombinant |
| 19SN951929 | 173 | 0 | 0.0% | A/G | A/G | A/C | A/G | C/G | A/G | Heterozygous |
| 19SN951986 | 125 | 0 | 0.0% | A/G | A/A | A/C | A/A | C/C | A/A | Heterozygous |
| 19SN951924 | 228 | 0 | 0.0% | A/A | Z/Z | A/A | A/G | C/G | A/G | Unfavorable |
| 19SN951894 | 206 | 0 | 0.0% | A/G | A/A | A/C | A/G | C/G | A/G | Unfavorable |
| 19SN951950 | 187 | 0 | 0.0% | A/G | A/G | A/C | A/A | C/C | A/A | Heterozygous |
| RWKS | 280 | 0 | 0.0% | A/A | A/A | A/A | A/A | C/C | A/A | Unfavorable |

| | | | | | | | | |
|---|---|---|---|---|---|---|---|---|
| Z21S | not tested | presumed 0 | G/G | A/A | C/C | G/G | C/C | A/A |

To verify the importance of this QTL in maize transformability, a diverse set of plant materials with a Normal A cytotype were evaluated for transformation performance and genotyped for this QTL. The five least transformable lines (TF<0.5%) did not have the favorable genotypes for all of these markers. In contrast, all of the lines that had TFs above 13% had the favorable genotypes for all of the markers or did not have enough data (see Table 22). All of the lines shown were homozygous for the markers, which is unsurprising as they are inbred plant lines.

One hundred and seventy two markers were evaluated in the chromosome 3 QTL interval, and a genotyping call was made based on whether the SNP present agreed with the SNP from SYN-INBC34. Table 23 lists the additional markers evaluated in the chromosome 3 QTL interval, along with genomic coordinates in the B73v5 reference genome and the genotype of each marker in SYN-INBC34 (i.e., in the TF allele at the qCYTO-A_TF3.1 TF-QTL). Any mismatches between a given variety and SYN-INBC34 were counted, and the total number of mismatches in the QTL region is presented in Table 22. From this data, an interpretation note was made (“Favorable” refers to at least 85% or at least 95% agreement with SYN-INBC34). Note that the highly transformable varieties all had the favorable allele, while the non-transformable varieties tended to not have the favorable allele.

TABLE 22
Transformation rate testing and genotyping data for the chromosome
3 QTL for a diversity of Normal A variety maize lines from
both non-stiff stalk and stiff stalk germplasm.

| | | | | # of Mismatches | Interpretation/ |
|---|---|---|---|---|---|
| Variety | Explants | Events | TF | to SYN-INBC34 | notes |
| SYN-INBM16 | 400 | 0 | 0 | 113/172 | Unfavorable |
| SYN-INBN17 | 298 | 0 | 0 | 0/41 for the first | Unfavorable |
| | | | | 41 until AXM9959 | |
| | | | | (@~25 Mb), then | |
| | | | | 72/131 | |
| SYN-INBO18 | 300 | 0 | 0 | 80/172 | Unfavorable |
| SYN-INBP19 | 634 | 2 | 0.3 | 83/172 | Unfavorable |
| SYN-INBQ20 | 576 | 4 | 0.7 | 0/58 for the first | Recombinant @ |
| | | | | 58 until AXM458 | 3′ end |
| | | | | (@~29 Mb), then | |
| | | | | 66/114 | |
| SYN-INBS21 | 250 | 2 | 0.8 | 86/172 | Unfavorable |
| SYN-INBA12 | 2379 | 34 | 1.4 | 95/172 | Unfavorable |
| SYN-INBT22 | 6570 | 156 | 2.4 | 55/172 | Unfavorable |
| SYN-INBU24 | 2838 | 99 | 3.5 | 0/172 | Favorable |
| SYN-INBV25 | 1490 | 68 | 4.6 | 0/67 for the first | Favorable |
| | | | | 67 until AXM6245 | |
| | | | | (@~31 Mb), then | |
| | | | | 62/105 | |
| SYN-INBB23 | 535 | 25 | 4.7 | 66/172 | Unfavorable |
| SYN-INBW26 | 2690 | 167 | 6.2 | 1/172 | Favorable |
| SYN-INBX27 | 4803 | 370 | 7.7 | 105/172 | Unfavorable |
| SYN-INBY28 | 1320 | 119 | 9 | 0/88 for the first | Favorable |
| | | | | 88 markers until | |
| | | | | AXM4798 | |
| | | | | (@~37 Mb), | |
| | | | | then 45/84 | |
| SYN-INBZ29 | 2131 | 262 | 12.3 | 0/172 | Favorable |
| SYN-INBAA30 | 1084 | 137 | 12.6 | 0/52 for the first | Favorable, 3′ |
| | | | | 52 markers until | recombinant |
| | | | | AXM6243 | |
| | | | | (@~28 Mb), | |
| | | | | then 32/120 | |
| SYN-INBAB31 | 3779 | 477 | 12.6 | 0/172 | Favorable |
| SYN-INBAC32 | 3000 | 383 | 12.8 | 0/172 | Favorable |
| SYN-INBAD33 | 2806842 | 426643 | 15.2 | 0/172 | Favorable |
| SYN-INBAE35 | 720 | 144 | 20 | 0/172 | Favorable |
| SYN-INBAF36 | 2561 | 675 | 26.4 | 0/172 | Favorable |
| SYN-INBAG37 | 729 | 281 | 38.5 | 0/172 | Favorable |
| SYN-INBC34 | 1340 | 619 | 46.2 | 0/172 | Favorable |

TABLE 23
Additional markers evaluated in
the chromosome 3 TF-QTL interval.

| | | Location | SYN-INBC34 |
|---|---|---|---|
| Marker | Chromosome | (B73v5) | genotype |
| AXM9160314338013G/ | 3 | 14338719 | C/C |
| GAXM13055 | | | |
| AXM5023 | 3 | 14739707 | G/G |
| AXM14922 | 3 | 15640904 | G/G |
| AXM8839 | 3 | 15913372 | G/G |
| AXM370 | 3 | 16413750 | A/A |
| AXM8867 | 3 | 16413900 | A/A |
| AXM1625 | 3 | 16413990 | G/G |
| AXM13506 | 3 | 16414044 | A/A |
| AXM4412 | 3 | 16414254 | A/A |
| AXM3449 | 3 | 16957568 | C/C |
| AXM7950 | 3 | 16957664 | G/G |
| AXM7949 | 3 | 16958032 | A/A |
| AXM9161 | 3 | 17365780 | A/A |
| AXM10413 | 3 | 17549738 | A/A |
| AXM12909 | 3 | 17997280 | A/A |
| AXM9162 | 3 | 18166040 | G/G |
| AXM9163 | 3 | 18187484 | A/A |
| AXM6232 | 3 | 18647436 | G/G |
| AXM6233 | 3 | 18649732 | A/A |
| AXM934 | 3 | 18871362 | A/A |
| AXM5348 | 3 | 19707290 | G/G |
| AXM1323 | 3 | 19810620 | A/A |
| AXM3844 | 3 | 19820740 | C/C |
| AXM6235 | 3 | 20813686 | A/A |
| AXM11235 | 3 | 20999452 | G/G |
| AXM869 | 3 | 21004152 | G/G |
| AXM132 | 3 | 21008904 | G/G |
| AXM6236 | 3 | 21198226 | A/A |
| AXM8682 | 3 | 21323478 | A/A |
| AXM3022 | 3 | 21879448 | A/A |
| AXM2496 | 3 | 22051712 | C/C |
| AXM7951 | 3 | 22052548 | A/A |
| AXM10414 | 3 | 22277748 | G/G |
| AXM3023 | 3 | 22640936 | A/A |
| AXM6239 | 3 | 22673002 | A/A |
| AXM9164 | 3 | 22674272 | A/A |
| AXM9165 | 3 | 24395398 | A/A |
| AXM3450324407084A/ | 3 | 24859396 | G/G |
| AAXM8838 | | | |
| AXM9959 | 3 | 25354518 | G/G |
| AXM3024 | 3 | 25690814 | C/C |
| AXM3025 | 3 | 25808968 | A/A |
| AXM7952 | 3 | 25812382 | G/G |
| AXM5828 | 3 | 27138684 | A/A |
| AXM10417 | 3 | 27140574 | G/G |
| AXM10418 | 3 | 27343412 | G/G |
| AXM6241 | 3 | 27356728 | A/A |
| AXM6242 | 3 | 27426036 | A/A |
| AXM10419 | 3 | 27834516 | A/A |
| AXM14975 | 3 | 28238924 | G/G |
| AXM6243 | 3 | 28455532 | G/G |
| AXM10420 | 3 | 28467692 | A/A |
| AXM8854 | 3 | 28626512 | G/G |
| AXM7953 | 3 | 28628344 | A/A |
| AXM9167 | 3 | 28669234 | G/G |
| AXM3026 | 3 | 28710708 | C/C |
| AXM870329092988A/ | 3 | 29675612 | G/G |
| AAXM498 | | | |
| AXM6244 | 3 | 30519364 | G/G |
| AXM1324 | 3 | 30691728 | A/A |
| AXM8625 | 3 | 30978854 | A/A |
| AXM8626 | 3 | 30980042 | A/A |
| AXM10421 | 3 | 31046284 | G/G |
| AXM10422 | 3 | 31082950 | G/G |
| AXM11461 | 3 | 31149944 | A/A |
| AXM6245 | 3 | 31291772 | A/A |
| AXM6246 | 3 | 31623896 | G/G |
| AXM409 | 3 | 31637688 | A/A |
| AXM10423 | 3 | 31702984 | G/G |
| AXM1986 | 3 | 31838034 | A/A |
| AXM3291 | 3 | 32005100 | C/C |
| AXM6247 | 3 | 32097760 | A/A |
| AXM8865 | 3 | 32671358 | G/G |
| AXM871 | 3 | 32750672 | G/G |
| AXM7954 | 3 | 35057272 | G/G |
| AXM285 | 3 | 35200456 | A/A |
| AXM13047 | 3 | 35352788 | A/A |
| AXM1809 | 3 | 35356328 | C/C |
| AXM1808 | 3 | 35356344 | C/C |
| AXM1737 | 3 | 35357320 | G/G |
| AXM3845 | 3 | 35448892 | C/C |
| AXM12060 | 3 | 35461896 | T/T |
| AXM5305 | 3 | 35462028 | A/A |
| AXM7955 | 3 | 36558100 | A/A |
| AXM7956 | 3 | 36558692 | A/A |
| AXM9784 | 3 | 36672744 | G/G |
| AXM11671 | 3 | 37806864 | G/G |
| AXM4798 | 3 | 37807620 | A/A |
| AXM10048 | 3 | 37807984 | A/A |
| AXM4575 | 3 | 37808092 | G/G |
| AXM3452 | 3 | 38519576 | C/C |
| AXM10426 | 3 | 38779764 | A/A |
| AXM676 | 3 | 38785376 | A/A |
| AXM144 | 3 | 38862600 | A/A |
| AXM7958 | 3 | 38953312 | A/A |
| AXM7957 | 3 | 38960964 | G/G |
| AXM3027 | 3 | 38961232 | A/A |
| AXM3028 | 3 | 39045360 | C/C |
| AXM9822 | 3 | 39049120 | G/G |
| AXM3722 | 3 | 39320176 | C/C |
| AXM9169 | 3 | 39322240 | G/G |
| AXM12965 | 3 | 39396080 | A/A |
| AXM11 | 3 | 39648640 | C/C |
| AXM13414 | 3 | 39912984 | C/C |
| AXM5267 | 3 | 39913008 | G/G |
| AXM6248 | 3 | 40862720 | A/A |
| AXM6249 | 3 | 40876556 | A/A |
| AXM6250 | 3 | 42357728 | G/G |
| AXM3029 | 3 | 42553584 | A/A |
| AXM13302 | 3 | 42679580 | G/G |
| AXM7959 | 3 | 42826796 | G/G |
| AXM6251 | 3 | 42826820 | A/A |
| AXM3030 | 3 | 42936224 | C/C |
| AXM2823 | 3 | 43323964 | A/A |
| AXM8681 | 3 | 43326200 | A/A |
| AXM593 | 3 | 43702296 | G/G |
| AXM3031 | 3 | 44580704 | C/C |
| AXM1327 | 3 | 44760860 | A/A |
| AXM7962 | 3 | 44763836 | G/G |
| AXM6252 | 3 | 44773872 | A/A |
| AXM14378 | 3 | 44812172 | C/C |
| AXM10427 | 3 | 45429580 | A/A |
| AXM6253 | 3 | 45760672 | A/A |
| AXM280 | 3 | 45761036 | A/A |
| AXM6254 | 3 | 47325636 | A/A |
| AXM9172 | 3 | 47386428 | G/G |
| AXM9173 | 3 | 48600904 | A/A |
| AXM2501 | 3 | 48716792 | C/C |
| AXM872 | 3 | 50321164 | A/A |
| AXM3846 | 3 | 51064844 | A/A |
| AXM757 | 3 | 51120380 | A/A |
| AXM9174 | 3 | 52093972 | A/A |
| AXM10428 | 3 | 52510540 | A/A |
| AXM13114 | 3 | 53036960 | A/A |
| AXM10429 | 3 | 53438152 | G/G |
| AXM6255 | 3 | 53554544 | A/A |
| AXM12985 | 3 | 53777484 | G/G |
| AXM3848 | 3 | 53863872 | A/A |
| AXM7965 | 3 | 53864056 | A/A |
| AXM1328 | 3 | 53869128 | A/A |
| AXM6256 | 3 | 54002792 | A/A |
| AXM2502 | 3 | 54023072 | C/C |
| AXM10430 | 3 | 54060920 | G/G |
| AXM3736 | 3 | 54061120 | C/C |
| AXM6257 | 3 | 55352672 | G/G |
| AXM2504 | 3 | 56948728 | A/A |
| AXM628 | 3 | 57051432 | C/C |
| AXM9175 | 3 | 57538200 | A/A |
| AXM9176 | 3 | 58039744 | A/A |
| AXM7456 | 3 | 58288984 | A/A |
| AXM2505 | 3 | 58292876 | A/A |
| AXM2362 | 3 | 58484552 | C/C |
| AXM14092 | 3 | 58484620 | C/C |
| AXM564 | 3 | 58484764 | C/C |
| AXM9177 | 3 | 58540328 | A/A |
| AXM7631 | 3 | 58718560 | A/A |
| AXM2177 | 3 | 58719312 | A/A |
| AXM13524 | 3 | 58719384 | G/G |
| AXM13134 | 3 | 58920420 | G/G |
| AXM14444 | 3 | 59698344 | A/A |
| AXM2506 | 3 | 59857832 | A/A |
| AXM6259 | 3 | 60050424 | A/A |
| AXM2819 | 3 | 60061320 | C/C |
| AXM6260 | 3 | 60271872 | A/A |
| AXM9178 | 3 | 60746916 | A/A |
| AXM14448 | 3 | 60953728 | A/A |
| AXM10431 | 3 | 60956976 | A/A |
| AXM1330 | 3 | 64699880 | G/G |
| AXM873 | 3 | 66279428 | G/G |
| AXM9922 | 3 | 66376932 | A/A |
| AXM3032 | 3 | 67404376 | A/A |

### Example 2. Transformation Rate and HI-Edit Testing

At least forty seed from individual F6 generation ear seed lots of the SYN-INBB23×RWKS derived plant materials shown in Table 24 were planted for transformation in the greenhouse at the Syngenta Biotechnology Innovation Center located in Research Triangle Park, North Carolina in December 2020.

TABLE 24
SYN-INBB23 × RWKS/Z21S//RWKS derived plant
materials planted for transformation rate testing.

| | F5 Plant | | Cas12+ Events/ | |
|---|---|---|---|---|
| Material | Material ID | Vectors | embryos initiated | TF Rate |
| RWKS/Z21/RWKS | control | 26258 + 24288 | 0/800 | 0.00% |
| 20BD917228 | 19SN952821 | 26258 | 7/800 | 0.90% |
| | | 26258 + 24288 | 29/450 | 6.40% |
| 20BD917233 | 19SN952822 | 26258 | 17/1100 | 1.50% |
| | | 26258 + 24288 | 36/450 | 8.00% |
| 20BD917237 | 19SN952871 | 26258 | 0/750 | 0.00% |
| | | 26258 + 24288 | 4/1200 | 0.30% |
| 20BD917621 | 19SN952763 | 26258 + 24288 | 0/938 | 0.00% |
| 20BD917284 | 19SN953098 | 26258 + 24288 | 8/1872 | 0.40% |

Separately, about 40 pooled seed from four F5 generation ears of the SYN-INBC34×RWKS plant materials shown in Table 25 were planted for transformation in the greenhouse at the same facility, in January of 2021.

TABLE 25
SYN-INBC34 × RWKS/Z21S//RWKS derived plant
materials planted for transformation rate testing.

| | F4 Plant | | Cas12+ Events/ | |
|---|---|---|---|---|
| Code | Material ID | Vectors | embryos initiated | TF rate |
| 20ALL1134VG_MM | 19SN952454 | 26258 + 24288 | 22/300 | 7.3% |
| 20ALL1134VH_MM | 19SN952019 | | 4/325 | 1.2% |
| 20ALL1134VI_MM | 19SN951958 | | 9/436 | 2.1% |
| 20ALL1134VJ_MM | 19SN951924 | | 15/750 | 2.0% |
| 20ALL1134VL_MM | 19SN952072 | | 32/470 | 6.8% |
| 20ALL1134VK_MM | 19SN952196 | 26258 | 62/300 | 20.7% |

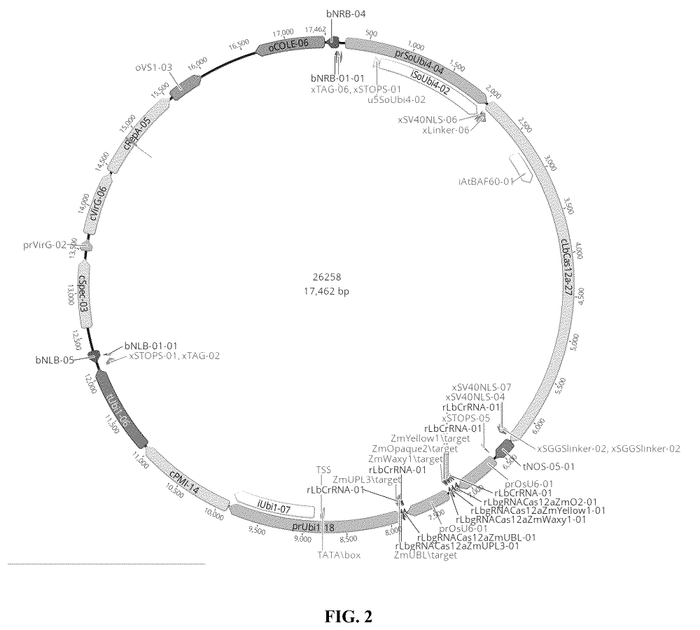

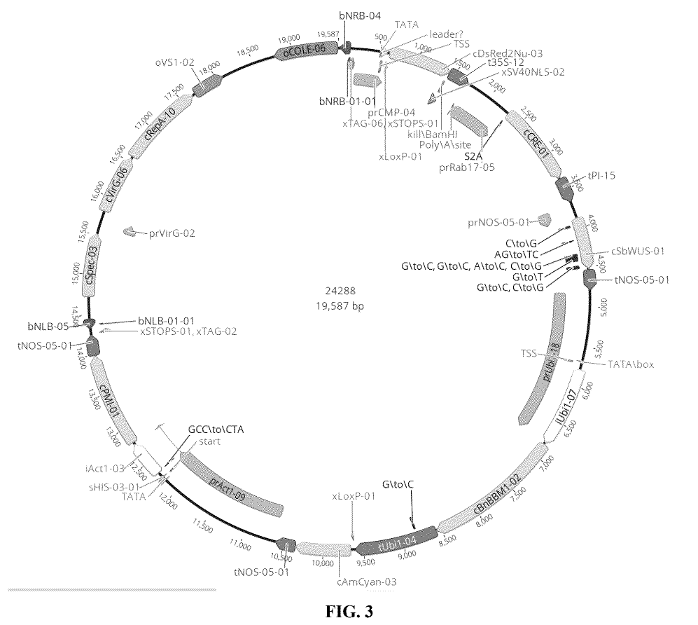

A binary construct, vector ID #26258 (FIG. 2; SEQ ID NO: 171) was built for transformation of F7-generation immature embryos from these plant materials. The vector comprises a phosphomannose isomerase (PMI) selectable marker cassette, as well as a Clustered Regularly-Interspaced Short Palindromic Repeats (CRISPR)—Cas12a cassette, and two cassettes containing Cas12a guide RNAs designed to target the following genes and sequences: Opaque2 on chromosome 7 (Zm00001d018971, CTGTATCTCGAGCGTCTGGCTGA; SEQ ID NO: 172), Waxy1 on chr 9 (Zm00001d045462, GGGAAAGACCGAGGAGAAGATCT; SEQ ID NO: 173), Yellow Endosperml on chr 6 (Zm00001d036345, CTATCTTATCCTAAAGATGGTGG; SEQ ID NO: 174), E3 ubiquitin ligase2 on chr 2 (Zm00001d004139, GGAGGGAAAAGGTGTCTGAGGC; SEQ ID NO: 175), and a putatitive ubiquitin-protein ligase on chr 5 (Zm00001d014920, GGAAGGAAAAGGTATCTGAAGG; SEQ ID NO: 176). The CRISPR/LbCas12a guide RNAs included a direct repeat of Lachnospiraceae bacterium ND2006 LbCrRNA. Note that a Cas9 cassette (which would require the use of different guideRNAs and multiplexing methods) could also be used instead of Cas12a (indeed, in U.S. patent Ser. No. 10/519,456 to Q. Que and T. Kelliher, U.S. patent Ser. No. 10/285,348 Q. Que and T. Kelliher, as well as Kelliher, T. et al., One step genome editing of elite crop germplasm (2019) Nature Biotechnology Volume 37, pages 287-292 a Cas9 vector was used for HI-Edit based genome editing). In addition to transforming 4 transformable plant materials (19SN952821, 19SN952822, 19SN952871, and 19SN952196) with this vector using a standard transformation protocol (outlined above in Example 1) the embryos from the first three of these lines plus seven other lines with high HIR and seed set (19SN952763, 19SN953098, 19SN952454, 19SN952019, 19SN951958, 19SN951924, 19SN952072) were co-transformed with vector 24288 (FIG. 3; SEQ ID NO: 177), which carried a Sorghum bicolor WUSCHEL cassette (cSbWUS-01; SEQ ID NO: 178) and a Brassica napus BABYBOOM1 cassette (cBnBBM1-02; SEQ ID NO: 179) to boost the number of transformants in certain varieties, a drought-inducible CRE-LOX excision system to enable removal of the WUS, CRE, and BBM1 cassettes after rooting. The transformation frequencies of all of these experiments are reported in tables 24 and 25.

The following developmental other genes may also be used to increase transformation frequency: BBM, BBM-WOX5, WOX5 (see, for example, PCT/US2020/045573, incorporated herein by reference). For reference, BBM, WUS2 or BBM-WUS2 assisted transformation has been validated in maize (Lowe et al. (2016) Morphogenic Regulators Baby boom and Wuschel Improve Monocot Transformation, Plant Cell 28, 1998-2015; Hoerster, et al. (2020) Use of non-integrating ZmWus2 vectors to enhance maize transformation, In Vitro Cellular & Developmental Biology; Mookkan et al., (2017) Selectable marker independent transformation of recalcitrant maize inbred B73 and sorghum P898012 mediated by morphogenic regulators BABY BOOM and WUSCHEL2, Plant Cell Reports 36:1477-1491). Alternatively, one may boost transformation using the GRF5 system (Kong et al. (2020) Overexpression of Transcription Factor Growth Regulating Factor5 Improves Transformation of Monocot and Dicot Species, Front. in Plant Sci, Vol. 11, Art. 572319) or by utilizing GRF4-GIF1 (J. M. Debernardi, et al. (2020), A GRF-GIF chimeric protein improves the regeneration efficiency of transgenic plants. Nature Biotechnology 38: 1274-1279).

At the same time as the transformation experiments in tables 24 and 25, the parental lines that went into transformation were also retested for haploid induction performance characteristics in the summer of 2021 (based on the average HIR and seed set from 3 ears from two tester lines each, and the high induction rate and seed set was generally confirmed for all lines that were submitted into the Cas12a 26258 transformation. The top line from the SYN-INBC23×RWKS (Iodent background) after the 2021 work was 20BD917233 (from 19SN952822), which maintained a very strong haploid induction rate and seed set characteristics (in 2021, the induction rate was 15.8%, with 207 seed per ear (total 32.3 haploids per ear), and which had superior performance and agronomic traits in field tests, and had a low but stable transformation rate (1.5%) that was enhanced by co-delivery via agrobacterium with vector 24288 (cSbWUS-01 and cBnBBM1-02 booster) to 8.0% (Table 24). In a separate experiment, the transformation rate of 20BD917233 via agrobacterium-mediated transformation of a Cas12a genome editing vector was 1.0% (two Cas12a-positive events out of 196 embryos), and this was enhanced using agrobacterium co-delivery with vector 25072 (SEQ ID NO: 180), which contains the WUSCHEL homeobox gene BdWOX5/7 (SEQ ID NO: 181) from Brachypodium distachyon (Bradi2g55270), driven by the maize Ubi1 promoter (SEQ ID NO: 182), to 7.0% (seventeen Cas12a-positive events out of 242 embryos).

For the SYN-INBC34×RWKS families, the top line was not clear. 20ALL1134VG_MM was a strong inducer and had a 7% transformation rate in the presence of BBM/WUS) but the field notes indicated yellowing and other agronomic issues. The most transformable germplasm was 20ALL1134VK_MM (20.7% TF) but it had a medium to low (about 6%) haploid induction rate in 2021 (Table 26). In order to identify a superior line from the SYN-INBC34×RWKS families (i.e. a new stiff stalk HI-Edit line with a high TF rate and strong HIR), we used the 2021 HIR data to select a new panel of elite inducers that were closely related to the transformable inducers we tested that were originally derived from 19SN952454 and 19SN952196 (they are F5 to F7 generation cousins/relative lines, derived from the same F4 family). Those bolded in Table 26 were in transformation and regenerating callus at the time of final submission of this document.

TABLE 26
Results of 2021 haploid induction rate performance of F5 to F7 derivatives
of the top two F4-derived transformable inducers, 19SN952454 and
19SN952196, from SYN-INBC34 x RWKS/Z21S//RWKS. Those bolded are
in transformation testing to evaluate the TF rate.

| | | | | | | haploids/ |
|---|---|---|---|---|---|---|
| male | Tester | Kernels | Haploids | Diploids | HIR % | ear |
| Control elite haploid | 1 | 481 | 83 | 398 | 17.3 | 27.7 |
| inducer | 2 | 619 | 147 | 426 | 25.7 | 52.9 |
| 20ALL1134VG_MM | 1 | 826 | 74 | 526 | 12.3 | 34.0 |
| (TF tested in 2021) | 2 | 508 | 99 | 409 | 19.5 | 33.0 |
| Relative#1 derived | 1 | 872 | 71 | 529 | 11.8 | 34.4 |
| from 19SN952454 | 2 | 945 | 97 | 503 | 16.2 | 50.9 |
| Relative#2 derived | 1 | 710 | 39 | 561 | 6.5 | 15.4 |
| from 19SN952454 | 2 | 775 | 85 | 491 | 14.8 | 38.1 |
| Relative#3 derived | 1 | 823 | 67 | 533 | 11.2 | 30.6 |
| from 19SN952454 | 2 | 813 | 82 | 518 | 13.7 | 37.0 |
| Relative#4 derived | 1 | 864 | 91 | 509 | 15.2 | 43.7 |
| from 19SN952454 | 2 | 583 | 91 | 452 | 16.8 | 32.6 |
| Relative#5 derived | 1 | 891 | 65 | 535 | 10.8 | 32.2 |
| from 19SN952454 | 2 | 875 | 92 | 508 | 15.3 | 44.7 |
| 20ALL1134VK_MM | 1 | 424 | 23 | 401 | 5.4 | 7.7 |
| (TF tested in 2021) | 2 | 219 | 17 | 202 | 7.8 | 5.7 |
| Relative #1 derived | 1 | 521 | 56 | 437 | 11.4 | 19.7 |
| from 19SN952196 | 2 | 609 | 76 | 494 | 13.3 | 27.1 |
| Relative #2 derived | 1 | 726 | 65 | 535 | 10.8 | 26.2 |
| from 19SN952196 | 2 | 371 | 62 | 309 | 16.7 | 20.7 |
| Relative #3 derived | 1 | 556 | 56 | 489 | 10.3 | 19.0 |
| from 19SN952196 | 2 | 684 | 93 | 496 | 15.8 | 36.0 |
| Relative #4 derived | 1 | 777 | 44 | 548 | 7.4 | 19.2 |
| from 19SN952196 | 2 | 763 | 50 | 550 | 8.3 | 21.2 |
| Relative #5 derived | 1 | 619 | 47 | 538 | 8.0 | 16.6 |
| from 19SN952196 | 2 | 501 | 51 | 450 | 10.2 | 17.0 |
| Relative #6 derived | 1 | 828 | 40 | 560 | 6.7 | 18.4 |
| from 19SN952196 | 2 | 644 | 68 | 504 | 11.9 | 25.5 |

Events were generated and tested for T-DNA insertions using TaqMan assay 2723 (which amplified the PMI-14 gene) and 3633 (which amplified the LbCas12a transgene). TO events were sent to the greenhouse, grown to flowering and self-pollinated to generate T1 seed. The TO generation plants were tested for edits to identify which T1 events have high CRISPR activity for use in HI-Edit trials. To assess target site editing, native allele TaqMan assays will be utilized which give a high PCR copy number for unedited “wild-type” alleles but which do not amplify or probe as strongly with edited alleles. Because we are using Cas12a, we expect the typical edits will be small deletions (commonly, Cas12a editing leads to deletions of 6 to 18 nucleotides in length, starting roughly 8 bp downstream from the PAM site). Therefore, the assays will have probe sequences covering this area that is deleted by Cas12a. For example, assay 3686 (TQ2817) was used for Waxy1: the probe GGTTTCAGGTTTGGGGAAAGA (SEQ ID NO:127) overlaps with gRNA target sequence GGGAAAGACCGAGGAGAAGATCT (SEQ ID NO 128). Table 27 shows the other assays primers.

TABLE 27
Vector 26258 Cas12a genome editing target genes, gRNA sequences
and assay ID, primer and probe sequences.

| Target | gRNA | | | | |
|---|---|---|---|---|---|
| Name | sequence | Assay | Fwd Primer | Rev Primer | Probe |
| Opaque2 | SEQ ID NO: | 3777 | FE12949: ATCGA | FE12950: | FE12951: |
| | 172 | | TCTGTCACTTGA | GTGAGCGGCTTT | TCTGGCTGA |
| | | | TTTTAATTAGAA | CCTGTATCT (SEQ | TTCTCTATT |
| | | | (SEQ ID NO: 183) | ID NO: 184) | (SEQ ID NO: |
| | | | | | 185) |
| Waxy1 | SEQ ID NO: | 3686 | FE12703: | FE12704: | FE12705: |
| | 173 | | TGTAGTCCGTTC | GGTTTCAGGTTT | CCGTAGATC |
| | | | CAGCGACA (SEQ | GGGGAAAGA | TTCTCCTC |
| | | | ID NO: 186) | (SEQ ID NO: 187) | (SEQ ID NO: |
| | | | | | 188) |
| Yellow | SEQ ID NO: | 3780 | FE12958: | FE12959: AGTAT | FE12960: |
| Endosperm1 | 174 | | GCTGACTGGTCT | GATGGCCATATA | CTAAAGATG |
| | | | CACCATCTCAT | TTTGCTATCTTA | GTGGTGGTG |
| | | | (SEQ ID NO: 189) | (SEQ ID NO: 190) | (SEQ ID NO: |
| | | | | | 191) |
| UPL | SEQ ID NO: | 3778 | FE12952: | FE12953: | FE12954: |
| (chr 5) | 176 | | TGGGACATTTG | GTCTAAGGGCCT | CTGAAGGAA |
| | | | GGAAGGAAA | GCTGACGA (SEQ | ACCTATCTC |
| | | | (SEQ ID NO: 192) | ID NO: 193) | (SEQ ID NO: |
| | | | | | 194) |
| E3 | SEQ ID NO: | 3779 | FE12952: | FE12956: | FE12957: |
| UPL2 | 175 | | TGGGACATTTG | CTGACTGCCTGA | TTGGTATGTT |
| (chr 2) | | | GGAAGGAAA | TGACGCA (SEQ | TGCCTCAGA |
| | | | (SEQ ID NO: 195) | ID NO: 196) | (SEQ ID NO: |
| | | | | | 197) |

Several TO events that have a single copy of the Cas12a vector T-DNA insertion and that were backbone-free were produced, and these events, along with others that had multiple copies including some backbone, were self-pollinated to make T1 seed. T1 plants homozygous for the CRISPR T-DNA (i.e. they were genotyped using TaqMan assays to determine the zygosity of the genome editing transgene, and they had at least two copies of the Cas12a and guide RNA editing machinery stably transformed, with single or multiple insertion events) were selected to be self-pollinated to generate T2 seed (Table 28).

TABLE 28
Representative events and their Taqman data in the elite HI-Edit inducer lines.

| Event | Line | Vector | BB | Waxy | O2 | UPL | UPL2 | Cas12a | BnBBM |
|---|---|---|---|---|---|---|---|---|---|
| MZKE211217A019A | 20ALL1134VK_MM | 26258 | 3 | 0 | 2 | 0 | 0 | 2 | N/A |
| MZKE211217A067A | 20ALL1134VK_MM | 26258 | 0 | 0 | 2 | 0 | 0 | 1 | N/A |
| MZKE211217A071A | 20ALL1134VK_MM | 26258 | 0 | 0 | 2 | 0 | 1 | 1 | N/A |
| MZKE211217A103A | 20ALL1134VK_MM | 26258 | 0 | 0 | 2 | 0 | 0 | 1 | N/A |
| MZKE211217A116A | 20ALL1134VK_MM | 26258 | 3 | 0 | 0 | 0 | 0 | 1 | N/A |
| MZKE211217A122A | 20ALL1134VK_MM | 26258 | 2 | 0 | 2 | 1 | 0 | 1 | N/A |
| MZKE211216A057A | 20ALL1134VG_MM | 26258; 24288 | 0 | 0 | 2 | 1 | 0 | 1 | 0 |
| MZKE211216A075A | 20ALL1134VG_MM | 26258; 24288 | 0 | 0 | 2 | 0 | 1 | 1 | 0 |
| MZKE211402B045A | 20ALL1134VL_MM | 26258; 24288 | 4 | 0 | 2 | 0 | 0 | 6 | 0 |

Column named “BB” refers to Agrobacterium backbone; “O2” refers to Opaque2; “UPL” refers to a putatitive ubiquitin-protein ligase on chromosome 5; “UPL2” refers to E3 ubiquitin ligase 2 on chromosome 2.

The resulting T2 plants are fixed homozygous for the editing machinery (T-DNA) (again these may be single, or, optionally multi-copy). At any of the T0 or T1 or T2 generations, the T-DNA+ plants may be outcrossed onto any maize inbred line to conduct “HI-Edit.” In this experiment, the T2 seed will be outcrossed onto a diversity of maize varieties including stiff stalk lines such as NP2222 and SYN-INBD45 (a non-stiff stalk iodent line), SYN-INBE56 (a non-stiff stalk line), SYN-MNF67 (a stiff stalk line), SYN-MNG78 (a non-stiff stalk iodent line), SYN-INBH89 (a non-stiff stalk iodent line, SYN-INBI90 (a non-stiff stalk iodent line), SYN-INBJ13 (a non-stiff stalk Mol7—like line), and SYN-INBK14 (a tropical line). Haploids will be identified and tested for edits according to the process outlined in (U.S. Pat. No. 10,285,348; Kelliher, T. et al., One step genome editing of elite crop germplasm (2019) Nature Biotechnology 37(3):287-292). Edited haploid plants from the temperate, tropical, subtropical or other germplasm will then be identified, doubled and grown to maturity and self-pollinated to generate edited DH seed (pure inbred edited lines) which will be used for additional breeding and seed production processes. All DH and DH1 generation lines and plants will be evaluated to confirm the existence of homozygous target-site edits and the lack of the CRISPR transgene (which should have been eliminated during the haploid induction process).

Because these events all derive from the new haploid inducer lines fixed for at least matl, the qhir8 HI allele, and R1-SCM2, they should have a robust haploid induction rate, and with the addition of the transgene, should be able to achieve an efficient level of HI-Edit in many maize lines. In this experiment, it is feasible to conduct HI-Edit using the pollen from TO events. However, it may be preferable to conduct HI-Edit in Homozygous T1 and T2 plants because in these plants, every pollen grain will carry the ability to HI-Edit: all will have haploid inducer alleles for genes/QTLs and will all carry a CRISPR transgene. If TO pollen is used, though the haploid inducer loci will be present in every pollen grain, the CRISPR machinery will not: for single-copy TO events, only 50% of pollen will have the CRISPR transgene, and for two-copy or higher events, it is likely that more than 50% of the pollen will have the transgene, depending on segregation.

During the HI-Edit pollinations, the female lines to be edited are non-haploid inducer lines (they have homozygous wild-type alleles for MATL and qhir8 and R1-SCM2 loci and lack CRISPR-Cas genome editing transgenes). Progeny embryos will be extracted from the cross-pollinated ears into petri dishes in the lab and will then be subjected to several assays to determine which are edited haploids. Progeny seed may also be grown to maturity, at which point the haploids may be identified by examining the embryo color and then germinated in soil. In the planned lab-based method, the embryos will be extracted, plated and then scored as diploid hybrids if they exhibit the purple color (coming from the action of the R1-SCM2 allele) or as haploids if they are cream colored. Other color markers, such as R1-nj, may be used in the alternative. See generally, Vijay Chaikam, et al. (2015), “Analysis of effectiveness of R1-nj anthocyanin marker for in vivo haploid identification in maize and molecular markers for predicting the inhibition of R1-nj expression,” Theor. Appl. Genet. 128(1):159-171. This is because the diploid hybrids carry the dominant R1-SCM2 allele from the male haploid inducer pollen-donor line, whereas the haploids are only comprised of a maternal genome and thus do not have the R1-SCM2 allele from the inducer line (i.e., the male genome is missing). Because the R1-SCM2 trait expresses in seeds and even in some parts of the plant, haploids may also be identified by the lack of color in the embryo in the mature stage or seedling stages. In many cases, depending on the amount of light the ear and developing kernels received during the early seed maturation phase after pollination, the diploid hybrid embryos are not purple upon extraction, but need to be exposed to light for anywhere from 2 to 36 hours before they turn purple (the light activates the anthocyanin pathway). The embryos will be extracted 13-22 days after pollination (DAP), though extraction between 10-25 DAP is theoretically possible, exposed to light for 16 to 24 hours, haploids will be kept; and diploids will be discarded. During the light treatment, embryos will be contacted with a chromosomal doubling agent such as colchicine (preferred), trifluralin, or another chromosome doubling agent. See, e.g., U.S. Patent Application Publication No. US2004/0210959 by C. L. Armstrong et al., incorporated herein by reference. Alternatively, a chromosome doubling agent may be applied to isolated embryos during germination or to seedlings of haploids germinated in soil. In the planned experiment, putative haploid embryos (cream colored) will be germinated in phytatrays and generate roots and leaves. After six to fourteen days of growth, small leaf samples will be taken to determine which of the putative haploids are edited. Again, TaqMan assays or typical PCR assays will be used to assess whether the target sites are edited in these putative haploids. Presence of mutations at the target site can be checked by sequence analysis (DNA sequencing), by marker analysis, or even by visual phenotype, depending on the gene target. As there is only one copy of the DNA to mutate in haploid plants, recessive phenotypes should display, so that could be another way to identify the haploids that were edited. At the same time the TaqMan assays for the target sites are being run, the putative haploids will be confirmed as true haploids by virtue of TaqMan assays designed to detect the CRISPR-Cas editing transgene and the haploid inducer markers from the male parent—true haploids will lack all of these genes or alleles (the markers will show up as wild-type or not present). Table 29 shows an example of an edited haploid marker outcome. Table 29. Example editing outcomes indicating genotyping results for haploids, edited haploids, and false haploids.

TABLE 29
Example editing outcomes indicating genotyping results for haploids, edited haploids, and false haploids.

| | Chrom. | | | | | | | gRNA | |
|---|---|---|---|---|---|---|---|---|---|
| | 1 | 1 | 9 | 9 | 10 | 10 | T-DNA | Target | |
| | Assay | | | | | | | | |
| | SM7252 | SM7246 | SM4849 | SM0956BQ | SM0954BQ | SM0953BQ | 3633 | 3686 | |
| | Gene / QTL | | | | | | | | |
| | matl | matl | qhir8 | qhir8 | R1-SCM2 | R1-SCM2 | Cas12a | Waxy | Call |
| Diploid | DI | CG | AT | AG | AC | AG | 1 | 0 | diploid |
| Control | | | | | | | | | |
| Putative | DD | CC | GG | AA | AA | GG | 0 | 2 | haploid |
| haploid 2 | | | | | | | | | |
| Putative | DD | CC | GG | AA | AA | GG | 0 | 2 | haploid |
| haploid 3 | | | | | | | | | |
| Putative | DD | CC | GG | AA | AA | GG | 0 | 0 | edited |
| haploid 4 | | | | | | | | | haploid |
| Putative | DD | CC | GG | AA | AA | GG | 0 | 2 | haploid |
| haploid 5 | | | | | | | | | |
| Putative | DI | CG | AG | AG | AC | AG | 1 | 0 | false |
| haploid 6 | | | | | | | | | haploid |

The putative edited haploids will be identified by the target site assays that do not amplify the WT allele as strongly as an unedited control, i.e. putative edited haploids will give either a “0” or “1” result for the “wild type” allele compared to a “2 copy” read for the unedited control. At the same time, it is expected that all of the haploids will have homozygous wild-type genotypes for the MATL, qhir8, and R1-SCM2 (TaqMan marker assays SM7252 and SM4849 at least), and will be ‘null’ for the transgenic Cas12a assay 3633, meaning they do not have the inducer alleles or editing machinery provided by the male parent and, if they are edited, that they were edited prior to male genome elimination and haploid induction.

In this experiment, it is also expected that some of the cream-colored embryos that we germinate and sample for TaqMan are false haploids, either due to color inhibition (i.e. the R1-SCM2 marker is not expressed or the purple color does not develop), partial (incomplete) male genome elimination (i.e. the embryos are chimeras or aneuploids partially lacking the inducer DNA) or pollen contamination (from female self-pollination or other pollen). Embryos produced by pollen contamination will not be edited. If false-positive haploids are edited (based on the target site assay) they will have a distinct pattern for other assays (the inducer allele of MATL, qhir8, R1-SCM2, or the CRISPR T-DNA transgene will be amplified) and can thus be identified and sorted away from true edited haploids.

Ploidy analysis via flow cytometry will also be performed on any putative edited haploid seedlings using leaf tissue in a ploidy analyzer to confirm the plant's status as a haploid. At this time, the plant may potentially be a doubled haploid (due to spontaneous or induced genome doubling), which in the flow cytometry results, would read the same as a diploid: the genetic markers are therefore critical to clarify which putative haploid (cream colored embryos) germinate into young plants that are edited but lack the inducer genome and editing machinery: these are the true edited haploids.

It is expected that out of one thousand embryos isolated from each female “elite” line from the crosses by the HI-Edit pollen, approximately 100 to 200 will be haploids, and among those haploids, between 0 and 100 will be edited at the guide RNA target sites. Typically, the efficiency of the editing of haploids is lower than a typical transformed plant because the CRISPR transgene and Cas protein-guideRNA complexes are only in the same nucleus as the female “elite” genome for a short period of time after fertilization but prior to the natural elimination of the haploid inducer DNA during haploid induction: male genome elimination may occur before, during, or within hours or days after fertilization. In past HI-Edit efforts (see U.S. patent Ser. No. 10/519,456 to Q. Que and T. Kelliher, U.S. patent Ser. No. 10/285,348 Q. Que and T. Kelliher, and Kelliher, T. et al. 2019. One Step Genome Editing of Elite Crop Germplasm, Nature Biotechnology Volume 37, pages 287-292), the haploid editing frequencies were observed to be between 0 and 10% in maize.

To determine the nature of the edits that occurred at the guide RNA target site of each target gene, the PCR fragments from the TaqMan-assays that give positive hits for editing will be sub-cloned through the use of a commercially-available TOPO Blunt IV kit, and at least four colonies for each subcloning reaction will be sequenced using forward and reverse primer Sanger sequencing. It is expected that small deletions will be identified in the PCR products from putatively edited plants at the Cas12a guide RNA cut site (starting about 8 to 10 basepairs downstream of the PAM site), as compared to the wild-type sequence. Edits in plants that gave a “0 copy” TaqMan result (i.e. no or very little PCR product amplification) for the guide RNA target site of interest may also be expected. Some haploids may be seen with more than one target site edited (see U.S. Pat. No. 10,285,348 and Kelliher, T. et al. 2019). The edits will be analyzed for the impact on the predicted protein sequence.

### Example 3. Breeding HI-NA Lines Using Backcrossing Strategy

The same process will be used as in Example 1, except instead of selfing for every generation (F2, F3, F4, through F5), backcrossing to the transformable backgrounds (e.g. SYN-INBC34 or SYN-INBB23) will be used, at one or more steps in the process, in order to increase the proportion of those genomes in the breeding populations. During backcrossing, the backcrosses will be made between those inbred lines (using them as either male or female plants) to breeding population plants carrying at least one copy of a HI allele at each of the critical haploid inducer loci (matl, qhir8, R1-SCM2 and optionally the color inhibitor). Marker assisted selection (genotyping) will be used to select for plants heterozygous for those alleles (either before or after the cross). After backcrossing one or more times, the resulting lines will again be screened for the genotype of those alleles, and plants heterozygous for all loci will be self-pollinated and then genotyping will be used once again to identify those plants that are homozygous for the haploid inducer alleles for most or all of those loci. An optional additional round of self-pollination will be performed to make all of those loci homozygous, and then the resulting lines (e.g. BC2F3) will be used for transformation rate and haploid induction rate testing. Lines that perform well in both phenotyping evaluations will be utilized for HI-Edit (e.g. those with a >5% transformation rate and >12% HIR).

### Example 4. Breeding Tropical or Subtropical HI-NA Lines

The same process as outlined in Examples 1 and 3 will be used to select for a transformable tropical haploid inducer line. Namely, a transformable, cytotype A tropical or subtropical line (e.g. SYN-INBA12) will be crossed as a female plant by pollen donated from RWKS/Z21S//RWKS or another haploid inducer line. An F2 or BC1 population (backcrossing to the tropical cytotype A line) will then be generated from the F1 plants that are generated in the original cross. Marker assisted selection will then be employed, utilizing the assays in Table 10, Table 11, and Table 12 (or other assays from those same genomic regions) to identify and select for F2 and/or BC1, BC2, F3 or later generation plants that comprise matl, the qhir8 HI allele, and the R1-SCM2 allele from the haploid inducer line and optionally for the wild-type allele for the chromosome 9 color inhibitor locus. At later generations (e.g. F4, F5, or BC1F3, or BC2F3, etc.) after fixation of the inducer alleles, the transformation rate and haploid induction rate will be tested and lines will be identified that perform well in both phenotyping evaluations (e.g. those with a >5% transformation rate and >12% HIR). These lines will then be used for HI-Edit transformation of Cas9 or Cas12 genome editing cassettes; the resulting TO, T1, T2, or later generation transformed lines will be used to cross to other tropical, sub-tropical, or temperate (stiff stalk or non-stiff stalk) lines to induce haploid induction and simultaneous genome editing (HI-Edit) as outlined in Example 2 (e.g. SYN-INBD45 (a non-stiff stalk iodent line), SYN-INBE56 (a non-stiff stalk line), SYN-INBF67 (a stiff stalk line), SYN-INBG78 (a non-stiff stalk iodent line), SYN-INBH89 (a non-stiff stalk iodent line, SYN-INBI90 (a non-stiff stalk iodent line), SYN-INBJ13 (a non-stiff stalk Mol7—like line), SYN-INBK14 (a tropical line). Edited haploid tropical or subtropical or other germplasm plants will then be identified, doubled and grown to maturity for self-pollination to generate edited DH seed (pure inbred edited lines) which will be used for additional breeding and seed production processes.

### Example 5. Breeding HI-NA Lines without Selecting for R1-SCM2 or Chromosome 9 Color Inhibitor

The same process as outlined in Examples 1, 3, and 4 will be followed, except in this case, the R1-SCM2 gene (and chromosome 9 color inhibitor) will not be selected using marker assisted selection; only the matl and qhir8 HI allele markers will be selected. Selected lines of the F4, F5, F6, or BC3F2, BC4F2, or later generations, will be transformed with a Cas9 or Cas12 or other genome editing cassette to be used for HI-Edit (with guide RNAs designed to a trait target) as in Example 2, and will contain a cassette coding for a visible or fluorescent marker that expresses in seeds or embryos: this will be used to identify haploids. An example of a marker gene is the green fluorescent protein or any other fluorescent protein or visible marker (such as GUS) under control of, for example, a Zein promoter (which will confer high and specific expression in seeds, as described in, e.g., Y. Wu and J. Messing, 2012, Rapid Divergence of Prolamin Gene Promoters of Maize After Gene Amplification and Dispersal, Genetics, Vol. 192, 507-519).

It is noted that the choice of promoter driving expression of the stably transformed editing proteins system may have a large impact on the rate of editing in haploids. For instance, a weak promoter or an inducible promoter may not sufficiently drive expression of the editing system absent other environmental effects, and in those scenarios the editing rate in haploids may thus be low. A constitutive sugarcane promoter (prSoUbi4) will be used, but other promoters driving high or specific expression in the embryo sac, in the pollen, or in sperm cells might be more effective (see Table 8 and accompanying description above).

### Example 6. Breeding HI-NA Lines Via Direct Mutational Targeting of MATL and DMP

The same process as outlined in Examples 1, 3, and 4 will be used, except in this case, only the R1-SCM2 gene (and optionally the chromosome 9 color inhibitor) will be selected using marker assisted selection, and then the selected lines are transformed and genome edited using CRISPR cassettes targeting the MATL and DMP genes which can confer a higher (>7%) HIR. The breeding is therefore greatly simplified—the breeding goal is simply to introgress the color marker into the transformable background, and high transformation rates can likely be obtained in most introgressed plant materials. The advantage is that this is a much faster and cheaper breeding process. After generation of BC3F2, BC4F2, or later generations, selected lines are transformed with a Cas9 or Cas12 or other genome editing cassette containing guide RNAs that are designed to induce knockout mutations in the MATL and DMP genes using one of the guide RNAs in Table 30 (there are other guide RNAs that would work here). TO plants with edits in both of the target genes are identified and self-pollinated. T1 or T2 or later generation progeny lacking the Cas transgene but homozygous for the edited matl and dmp alleles are identified and used to confirm the high (7%-25%) HIR (with the haploid selection marker utilizing the R1-SCM2 color change). Later, the high induction rate and highly transformable lines are then transformed with a new CRISPR genome editing cassette to be used for HI-Edit (with guide RNAs designed to a trait target), as in Example 2.

TABLE 30
Guide RNAs designed to knock out the DMP and MATL genes.

| gRNA | Gene | Maize B73_v4/v5 ID | Enzyme | Sequence (5' to 3') | PAM site |
|---|---|---|---|---|---|
| 1 | DMP | GRMZM2G465053/ | Cas12a | CTCCCTGGGCACCTCGACGCCGA | TTTC |
| | | Zm00001d044822 | | (SEQ ID NO: 165) | |
| 2 | | | Cas9 | GGCCGGGAGTAGCATCTCGA | AGG |
| | | | | (SEQ ID NO: 166) | |
| 3 | | | Cas9 | ACTACGGCTTCGTCACGCCG | CGG |
| | | | | (SEQ ID NO: 167) | |
| 4 | MATL | GRMZM2G471240/ | Cas12a | CATGCAGAACTGCCCGCGCATCT | TTTA |
| | | Zm00001d029412 | | (SEQ ID NO: 168) | |
| 5 | | | Cas9 | GGGTCAACGTGGAGACAGGG | AGG |
| | | | | (SEQ ID NO: 169) | |
| 6 | | | Cas9 | CTTCCTGGAGGCCAGGCTGC | AGG |
| | | | | (SEQ ID NO: 170) | |

### Example 7. Creating HI-NA Lines without Breeding

The RWKS/Z21S//RWKS BC1 haploid inducer, or any other high performing haploid inducer line, will be transformed using the BBM or related elite line transformation technology as outlined in Example 2 to deliver CRISPR transgenes for the purposes of HI-Edit. All of the other steps in HI-Edit will be performed as in Example 2. The advantage here is that there is no breeding needed—and the very best performing haploid inducer lines (with all of the inducer genes and color marker) can be directly used for HI-Edit. In this example, the haploid inducer lines to be transformed do not have a normal A cytotype, and they have a baseline transformation frequency of less than 1%. As such, the BBM technology or another transformation boosting technology (see Example 2) will be needed to achieve sufficient transformation of the high performing haploid inducer line.

### Example 8. Creating HI-NA Lines Via Direct Mutational Targeting of MATL and DMP and Without Selecting for R1-SCM2 or Chromosome 9 Color Inhibitor

Any line (not a haploid inducer line, and one also lacking the color marker) will be transformed with a first construct comprising a Cas9 or Cas12 genome editing cassette and a guide RNA cassette designed to target the MATRILINEAL and DMP genes (as in Example 6). That line will then be used as a new HI-Edit line. Additional guides will be included in the first construct which can be used for HI-Edit, or, preferably, new transformations will be performed on CRISPR-free matl and dmp mutant T1 or T2 lines (as in Example 5) to bring in new genome editing constructs for the purpose of HI-Edit. Either way, the HI-Edit constructs will contain a cassette coding for a visible or fluorescent marker that expresses in seeds or embryos: this will be used to identify haploids. The advantage of this process is that any transformable line can be used for HI-Edit, so long as the matl and dmp edits are made and so long as there is a transgenic marker included so that haploids can easily be identified. An example of a marker gene is a green fluorescent protein under control of a Zein promoter (which will confer high and specific expression in seeds) as described in Example 5.

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

In the foregoing description, numerous specific details are set forth to provide a more thorough understanding of the present invention. However, it will be apparent to one of skill in the art that the invention described in this disclosure may be practiced without one or more of these specific details. In other instances, well-known features and procedures well known to those skilled in the art have not been described in order to avoid obscuring the invention. Embodiments of the disclosure have been described for illustrative and not restrictive purposes. Although the present invention is described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

1. A maize plant homozygous for a loss-of-function mutation in the patatin-like phospholipase A2α gene (MATL) and at least heterozygous for a HIT allele at at least one quantitative trait locus (QTL) associated with increased haploid induction (HI-QTL), wherein the maize plant has a normal A (“NA”) cytotype.

2. The maize plant of claim 1, wherein the maize plant is homozygous for the HI allele at the at least one HI-QTL.

3. The maize plant of claim 1, wherein the at least one HI-QTL is qhir8 on chromosome 9 (HI-QTL qhir8).

4. The maize plant of claim 3, wherein the HI allele at the HI-QTL qhir8 comprises a loss-of function mutation in the DUF679 domain membrane protein 7 (DMP) gene.

5. The maize plant of claim 1, wherein the maize plant is at least heterozygous for a TF allele at at least one QTL associated with increased transformation frequency (TF-QTL).

6. The maize plant of claim 5, wherein the maize plant is homozygous for the TF allele at the at least one TF-QTL.

7. The maize plant of claim 6, wherein the at least one TF-QTL is qCYTO-A TF3.1 on chromosome 3 (TF-QTL qCYTO-A_TF3.1).

8. The maize plant of claim 1, wherein the maize plant comprises a selectable marker.

9. The maize plant of claim 8, wherein the maize plant is homozygous for a selectable marker.

10. The maize plant of claim 9, wherein the selectable marker is any one of GUS, PMI, PAT, GFP, RFP, CFP, B1, CI, NPTII, HPT, ACC3, AADA, high oil content, R-navajo (R-nj), R1-scutellum (R1-SCM2), and/or an anthocyanin pigment.

11. The maize plant of claim 10, wherein the maize plant is homozygous for the R1-scutellum (R1-SCM2) allele at the R1 locus on chromosome 10.

12. The maize plant of claim 11, wherein the maize plant is at least heterozygous for a wild-type allele at a color inhibitor locus in the maize plant that corresponds to a color inhibitor locus located on chromosome 9 between position 8 Mb and 10 Mb in the B73v5 reference genome.

13. The maize plant of claim 1, wherein the maize plant is capable of expressing a DNA modification enzyme and optionally at least one guide nucleic acid.

14. The maize plant of claim 13, wherein the DNA modification enzyme is a site-directed nuclease selected from the group consisting of Cas9 nuclease, Cas12a nuclease, meganucleases (MNs), zinc-finger nucleases, (ZFNs), transcription-activator like effector nucleases (TALENs), dCas9-Fokl, dCas12a-FokI, chimeric Cas9-cytidine deaminase, chimeric Cas9-adenine deaminase, chimeric FEN-FokI, MegaTALs, a nickase Cas9 (nCas9), chimeric dCas9 non-Fokl nuclease, dCas12a non-Fokl nuclease, chimeric Cas12a-cytidine deaminase, and Cas12a-adenine deaminase.

15. The maize plant of claim 1, wherein the maize plant comprises one or more of a Non-Stiff Stalk germplasm, a Stiff Stalk germplasm, a Non-Stiff Stalk Iodent germplasm, a Non-Stiff Stalk Mo17-like germplasm, a Tropical germplasm, or a Subtropical germplasm.

16. The maize plant of claim 1, wherein the maize plant is derived from any of lines Stock 6, RWK, RWS, UH400, AX5707RS, NP2222, SYN-INBE56, SYN-INBB23, SYN-NBF67, SYN-INBC34, SYN-INBD45, SYN-INBG78, SYN-INBH89, SYN-INBI90, SYN-INBJ13, and/or SYN-INBK14.

17. A maize plant that is at least heterozygous for a TF allele at at least one quantitative trait locus (QTL) associated with increased transformation frequency (TF-QTL).

18. The maize plant of claim 17, wherein the maize plant is homozygous for the TF allele at the at least one TF-QTL.

19. The maize plant of claim 17, wherein the at least one TF-QTL is qCYTO-A_TF3.1 on chromosome 3 (TF-QTL qCYTO-A TF3.1).

20. The maize plant of claim 17, wherein the maize plant has a normal A (“NA”) cytotype.

21.-45. (canceled)


SEQUENCE LISTING (197 sequences)

SEQ ID NO: 1 - DNA, 19 bp, Artificial Sequence
  SM7246 F primer
gtaaagccacttgtcttag

SEQ ID NO: 2 - DNA, 16 bp, Artificial Sequence
  SM7246 R primer
ctcgctaggcagctct

SEQ ID NO: 3 - DNA, 13 bp, Artificial Sequence
  SM7246 FAM
cgccgcgtctctg

SEQ ID NO: 4 - DNA, 13 bp, Artificial Sequence
  SM7246 TET
cgccgcgtgtctg

SEQ ID NO: 5 - DNA, 1005 bp, Zea mays
  misc_feature (5)..(5): N = Adenine or Guanine or Cytosine or Thymine
  misc_feature (19)..(19): N = Adenine or Guanine or Cytosine or Thymine
  misc_feature (28)..(28): N = Adenine or Guanine or Cytosine or Thymine
  misc_feature (40)..(40): N = Adenine or Guanine or Cytosine or Thymine
  misc_feature (49)..(49): N = Adenine or Guanine or Cytosine or Thymine
  misc_feature (113)..(113): N = Adenine or Guanine or Cytosine or Thymine
  misc_feature (118)..(118): N = Adenine or Guanine or Cytosine or Thymine
  misc_feature (125)..(125): N = Adenine or Guanine or Cytosine or Thymine
  misc_feature (131)..(131): N = Adenine or Guanine or Cytosine or Thymine
  misc_feature (145)..(145): N = Adenine or Guanine or Cytosine or Thymine
  misc_feature (171)..(171): N = Adenine or Guanine or Cytosine or Thymine
  misc_feature (207)..(207): N = Adenine or Guanine or Cytosine or Thymine
  misc_feature (215)..(215): N = Adenine or Guanine or Cytosine or Thymine
  misc_feature (223)..(223): N = Adenine or Guanine or Cytosine or Thymine
  misc_feature (343)..(347): N = Adenine or Guanine or Cytosine or Thymine
  misc_feature (386)..(386): N = Adenine or Guanine or Cytosine or Thymine
  misc_feature (392)..(392): N = Adenine or Guanine or Cytosine or Thymine
  misc_feature (432)..(432): N = Adenine or Guanine or Cytosine or Thymine
  misc_feature (465)..(465): N = Adenine or Guanine or Cytosine or Thymine
  misc_feature (467)..(467): N = Adenine or Guanine or Cytosine or Thymine
  misc_feature (482)..(482): N = Adenine or Guanine or Cytosine or Thymine
  misc_feature (486)..(486): N = Adenine or Guanine or Cytosine or Thymine
  misc_feature (534)..(534): N = Adenine or Guanine or Cytosine or Thymine
  misc_feature (582)..(582): N = Adenine or Guanine or Cytosine or Thymine
  misc_feature (585)..(585): N = Adenine or Guanine or Cytosine or Thymine
  misc_feature (601)..(604): N = Adenine or Guanine or Cytosine or Thymine
  misc_feature (636)..(636): N = Adenine or Guanine or Cytosine or Thymine
  misc_feature (706)..(706): N = Adenine or Guanine or Cytosine or Thymine
  misc_feature (742)..(742): N = Adenine or Guanine or Cytosine or Thymine
  misc_feature (838)..(838): N = Adenine or Guanine or Cytosine or Thymine
  misc_feature (871)..(871): N = Adenine or Guanine or Cytosine or Thymine
  misc_feature (967)..(967): N = Adenine or Guanine or Cytosine or Thymine
cttcngatttgccgagcgnttttttttngcctagtgtctngcactaggnaaacttttgatttgccagtgttttgctttgcctagtggcggacactcggcaaatgactgatttncctantgtcttnctttgnccagcgtaacactnggcaaagaagatctttgcctagtgcncgtgaattcacactcggcaaaatatttagcgccagncaaagttngtctctcncgtagtgtcatcatatattttatactagtatgattggttctatagtcattcacggtagtccttcccgttcacgttttataaaaaataaagtaaaattatgtataagtgatggttcgaatnnnnntttggttatcaattttatattcaagaacatatatatatntttatnttttattaaaataaagtctacttatttacaagcatgtgantgacagtaaagccacttgtcttagatatcgtangncgcacatctagagcntctngggttgatggcagacacgcggcgcgcgagccttgttctcctctcctcngagagctgcctagcgagcccaccgagggcatcggcattgcttccttcnccngtcaccggttcgtacnnnnctccctgtctccacgttgaccctggacacccnctgtgccagcatccgctccccgatcccgacgagcgtccgcatgttctccggcgtcgccgcgtccacggtngccgcggcgccacggagcgagttgtcctggatgcgnaggtagtcgccgtcgctgtggagcgactggaacatcgcggcgacgtggatgtccaccaggtccgagctggccgccatgaagatgtcgatgatgggngccatgccgttgttgcggagccaccggcagatnccccaccgggagcactgccgcgccgtgtagaggccctgctcggacgtcgaccccgtcccgatggacagcaccaggaacctgcggcagttcgacggnttcactgggtacagctcctcggccttgtccttgctggc

SEQ ID NO: 6 - DNA, 1005 bp, Zea mays
  misc_feature (5)..(5): N = Adenine or Guanine or Cytosine or Thymine
  misc_feature (19)..(19): N = Adenine or Guanine or Cytosine or Thymine
  misc_feature (28)..(28): N = Adenine or Guanine or Cytosine or Thymine
  misc_feature (40)..(40): N = Adenine or Guanine or Cytosine or Thymine
  misc_feature (49)..(49): N = Adenine or Guanine or Cytosine or Thymine
  misc_feature (113)..(113): N = Adenine or Guanine or Cytosine or Thymine
  misc_feature (118)..(118): N = Adenine or Guanine or Cytosine or Thymine
  misc_feature (125)..(125): N = Adenine or Guanine or Cytosine or Thymine
  misc_feature (131)..(131): N = Adenine or Guanine or Cytosine or Thymine
  misc_feature (145)..(145): N = Adenine or Guanine or Cytosine or Thymine
  misc_feature (171)..(171): N = Adenine or Guanine or Cytosine or Thymine
  misc_feature (207)..(207): N = Adenine or Guanine or Cytosine or Thymine
  misc_feature (215)..(215): N = Adenine or Guanine or Cytosine or Thymine
  misc_feature (223)..(223): N = Adenine or Guanine or Cytosine or Thymine
  misc_feature (343)..(347): N = Adenine or Guanine or Cytosine or Thymine
  misc_feature (386)..(386): N = Adenine or Guanine or Cytosine or Thymine
  misc_feature (392)..(392): N = Adenine or Guanine or Cytosine or Thymine
  misc_feature (432)..(432): N = Adenine or Guanine or Cytosine or Thymine
  misc_feature (465)..(465): N = Adenine or Guanine or Cytosine or Thymine
  misc_feature (467)..(467): N = Adenine or Guanine or Cytosine or Thymine
  misc_feature (482)..(482): N = Adenine or Guanine or Cytosine or Thymine
  misc_feature (486)..(486): N = Adenine or Guanine or Cytosine or Thymine
  misc_feature (534)..(534): N = Adenine or Guanine or Cytosine or Thymine
  misc_feature (582)..(582): N = Adenine or Guanine or Cytosine or Thymine
  misc_feature (585)..(585): N = Adenine or Guanine or Cytosine or Thymine
  misc_feature (601)..(604): N = Adenine or Guanine or Cytosine or Thymine
  misc_feature (636)..(636): N = Adenine or Guanine or Cytosine or Thymine
  misc_feature (706)..(706): N = Adenine or Guanine or Cytosine or Thymine
  misc_feature (742)..(742): N = Adenine or Guanine or Cytosine or Thymine
  misc_feature (838)..(838): N = Adenine or Guanine or Cytosine or Thymine
  misc_feature (871)..(871): N = Adenine or Guanine or Cytosine or Thymine
  misc_feature (967)..(967): N = Adenine or Guanine or Cytosine or Thymine
cttcngatttgccgagcgnttttttttngcctagtgtctngcactaggnaaacttttgatttgccagtgttttgctttgcctagtggcggacactcggcaaatgactgatttncctantgtcttnctttgnccagcgtaacactnggcaaagaagatctttgcctagtgcncgtgaattcacactcggcaaaatatttagcgccagncaaagttngtctctcncgtagtgtcatcatatattttatactagtatgattggttctatagtcattcacggtagtccttcccgttcacgttttataaaaaataaagtaaaattatgtataagtgatggttcgaatnnnnntttggttatcaattttatattcaagaacatatatatatntttatnttttattaaaataaagtctacttatttacaagcatgtgantgacagtaaagccacttgtcttagatatcgtangncgcacatctagagcntctngggttgatggcagagacgcggcgcgcgagccttgttctcctctcctcngagagctgcctagcgagcccaccgagggcatcggcattgcttccttcnccngtcaccggttcgtacnnnnctccctgtctccacgttgaccctggacacccnctgtgccagcatccgctccccgatcccgacgagcgtccgcatgttctccggcgtcgccgcgtccacggtngccgcggcgccacggagcgagttgtcctggatgcgnaggtagtcgccgtcgctgtggagcgactggaacatcgcggcgacgtggatgtccaccaggtccgagctggccgccatgaagatgtcgatgatgggngccatgccgttgttgcggagccaccggcagatnccccaccgggagcactgccgcgccgtgtagaggccctgctcggacgtcgaccccgtcccgatggacagcaccaggaacctgcggcagttcgacggnttcactgggtacagctcctcggccttgtccttgctggc

SEQ ID NO: 7 - DNA, 20 bp, Artificial Sequence
  SM7252 F primer
ggcatcggcattgcttcctt

SEQ ID NO: 8 - DNA, 18 bp, Artificial Sequence
  SM7252 R primer
gggtgtccagggtcaacg

SEQ ID NO: 9 - DNA, 16 bp, Artificial Sequence
  SM7252 FAM
cagggagcgaggtacg

SEQ ID NO: 10 - DNA, 19 bp, Artificial Sequence
  SM7252 TET
agacagggaggtacgaacc

SEQ ID NO: 11 - DNA, 1000 bp, Zea mays
  misc_feature (13)..(13): N = Adenine or Guanine or Cytosine or Thymine
  misc_feature (18)..(18): N = Adenine or Guanine or Cytosine or Thymine
  misc_feature (25)..(25): N = Adenine or Guanine or Cytosine or Thymine
  misc_feature (31)..(31): N = Adenine or Guanine or Cytosine or Thymine
  misc_feature (45)..(45): N = Adenine or Guanine or Cytosine or Thymine
  misc_feature (71)..(71): N = Adenine or Guanine or Cytosine or Thymine
  misc_feature (107)..(107): N = Adenine or Guanine or Cytosine or Thymine
  misc_feature (115)..(115): N = Adenine or Guanine or Cytosine or Thymine
  misc_feature (123)..(123): N = Adenine or Guanine or Cytosine or Thymine
  misc_feature (243)..(247): N = Adenine or Guanine or Cytosine or Thymine
  misc_feature (286)..(286): N = Adenine or Guanine or Cytosine or Thymine
  misc_feature (292)..(292): N = Adenine or Guanine or Cytosine or Thymine
  misc_feature (332)..(332): N = Adenine or Guanine or Cytosine or Thymine
  misc_feature (365)..(365): N = Adenine or Guanine or Cytosine or Thymine
  misc_feature (367)..(367): N = Adenine or Guanine or Cytosine or Thymine
  misc_feature (382)..(382): N = Adenine or Guanine or Cytosine or Thymine
  misc_feature (386)..(386): N = Adenine or Guanine or Cytosine or Thymine
  misc_feature (401)..(401): N = Adenine or Guanine or Cytosine or Thymine
  misc_feature (434)..(434): N = Adenine or Guanine or Cytosine or Thymine
  misc_feature (482)..(482): N = Adenine or Guanine or Cytosine or Thymine
  misc_feature (485)..(485): N = Adenine or Guanine or Cytosine or Thymine
  misc_feature (532)..(532): N = Adenine or Guanine or Cytosine or Thymine
  misc_feature (602)..(602): N = Adenine or Guanine or Cytosine or Thymine
  misc_feature (638)..(638): N = Adenine or Guanine or Cytosine or Thymine
  misc_feature (734)..(734): N = Adenine or Guanine or Cytosine or Thymine
  misc_feature (767)..(767): N = Adenine or Guanine or Cytosine or Thymine
  misc_feature (863)..(863): N = Adenine or Guanine or Cytosine or Thymine
aatgactgatttncctantgtcttnctttgnccagcgtaacactnggcaaagaagatctttgcctagtgcncgtgaattcacactcggcaaaatatttagcgccagncaaagttngtctctcncgtagtgtcatcatatattttatactagtatgattggttctatagtcattcacggtagtccttcccgttcacgttttataaaaaataaagtaaaattatgtataagtgatggttcgaatnnnnntttggttatcaattttatattcaagaacatatatatatntttatnttttattaaaataaagtctacttatttacaagcatgtgantgacagtaaagccacttgtcttagatatcgtangncgcacatctagagcntctngggttgatggcaganacgcggcgcgcgagccttgttctcctctcctcngagagctgcctagcgagcccaccgagggcatcggcattgcttccttcnccngtcaccggttcgtacctccctgtctccacgttgaccctggacacccnctgtgccagcatccgctccccgatcccgacgagcgtccgcatgttctccggcgtcgccgcgtccacggtngccgcggcgccacggagcgagttgtcctggatgcgnaggtagtcgccgtcgctgtggagcgactggaacatcgcggcgacgtggatgtccaccaggtccgagctggccgccatgaagatgtcgatgatgggngccatgccgttgttgcggagccaccggcagatnccccaccgggagcactgccgcgccgtgtagaggccctgctcggacgtcgaccccgtcccgatggacagcaccaggaacctgcggcagttcgacggnttcactgggtacagctcctcggccttgtccttgctggcaagcatctttttggtgatctgcgtcatcgcaaccatcgtctacgcatgtgcaatcgggcgacatacataagcaacaattagcatcgaccttgtgggccg

SEQ ID NO: 12 - DNA, 1004 bp, Zea mays
  misc_feature (13)..(13): N = Adenine or Guanine or Cytosine or Thymine
  misc_feature (18)..(18): N = Adenine or Guanine or Cytosine or Thymine
  misc_feature (25)..(25): N = Adenine or Guanine or Cytosine or Thymine
  misc_feature (31)..(31): N = Adenine or Guanine or Cytosine or Thymine
  misc_feature (45)..(45): N = Adenine or Guanine or Cytosine or Thymine
  misc_feature (71)..(71): N = Adenine or Guanine or Cytosine or Thymine
  misc_feature (107)..(107): N = Adenine or Guanine or Cytosine or Thymine
  misc_feature (115)..(115): N = Adenine or Guanine or Cytosine or Thymine
  misc_feature (123)..(123): N = Adenine or Guanine or Cytosine or Thymine
  misc_feature (243)..(247): N = Adenine or Guanine or Cytosine or Thymine
  misc_feature (286)..(286): N = Adenine or Guanine or Cytosine or Thymine
  misc_feature (292)..(292): N = Adenine or Guanine or Cytosine or Thymine
  misc_feature (332)..(332): N = Adenine or Guanine or Cytosine or Thymine
  misc_feature (365)..(365): N = Adenine or Guanine or Cytosine or Thymine
  misc_feature (367)..(367): N = Adenine or Guanine or Cytosine or Thymine
  misc_feature (382)..(382): N = Adenine or Guanine or Cytosine or Thymine
  misc_feature (386)..(386): N = Adenine or Guanine or Cytosine or Thymine
  misc_feature (401)..(401): N = Adenine or Guanine or Cytosine or Thymine
  misc_feature (434)..(434): N = Adenine or Guanine or Cytosine or Thymine
  misc_feature (482)..(485): N = Adenine or Guanine or Cytosine or Thymine
  misc_feature (536)..(536): N = Adenine or Guanine or Cytosine or Thymine
  misc_feature (606)..(606): N = Adenine or Guanine or Cytosine or Thymine
  misc_feature (642)..(642): N = Adenine or Guanine or Cytosine or Thymine
  misc_feature (738)..(738): N = Adenine or Guanine or Cytosine or Thymine
  misc_feature (771)..(771): N = Adenine or Guanine or Cytosine or Thymine
  misc_feature (867)..(867): N = Adenine or Guanine or Cytosine or Thymine
aatgactgatttncctantgtcttnctttgnccagcgtaacactnggcaaagaagatctttgcctagtgcncgtgaattcacactcggcaaaatatttagcgccagncaaagttngtctctcncgtagtgtcatcatatattttatactagtatgattggttctatagtcattcacggtagtccttcccgttcacgttttataaaaaataaagtaaaattatgtataagtgatggttcgaatnnnnntttggttatcaattttatattcaagaacatatatatatntttatnttttattaaaataaagtctacttatttacaagcatgtgantgacagtaaagccacttgtcttagatatcgtangncgcacatctagagcntctngggttgatggcaganacgcggcgcgcgagccttgttctcctctcctcngagagctgcctagcgagcccaccgagggcatcggcattgcttccttcnccngtcaccggttcgtacctcgctccctgtctccacgttgaccctggacacccnctgtgccagcatccgctccccgatcccgacgagcgtccgcatgttctccggcgtcgccgcgtccacggtngccgcggcgccacggagcgagttgtcctggatgcgnaggtagtcgccgtcgctgtggagcgactggaacatcgcggcgacgtggatgtccaccaggtccgagctggccgccatgaagatgtcgatgatgggngccatgccgttgttgcggagccaccggcagatnccccaccgggagcactgccgcgccgtgtagaggccctgctcggacgtcgaccccgtcccgatggacagcaccaggaacctgcggcagttcgacggnttcactgggtacagctcctcggccttgtccttgctggcaagcatctttttggtgatctgcgtcatcgcaaccatcgtctacgcatgtgcaatcgggcgacatacataagcaacaattagcatcgaccttgtgggccg

SEQ ID NO: 13 - DNA, 17 bp, Artificial Sequence
  Assay 2826 TREM26 F primer
gcggatgctggcacaga

SEQ ID NO: 14 - DNA, 21 bp, Artificial Sequence
  Assay 2826 PM0033 F primer
gaacgtgtgttgggtttgcat

SEQ ID NO: 15 - DNA, 18 bp, Artificial Sequence
  Assay 2826 TREM26 R primer
gcattgcttccttcgcca

SEQ ID NO: 16 - DNA, 20 bp, Artificial Sequence
  Assay 2826 PM0033 R primer
tccagcaatccttgcacctt

SEQ ID NO: 17 - DNA, 16 bp, Artificial Sequence
  Assay 2826 FAM
cagggaggtacgaacc

SEQ ID NO: 18 - DNA, 24 bp, Artificial Sequence
  Assay 2826 TET
tgcagcctaaccatgcgcagggta

SEQ ID NO: 19 - DNA, 17 bp, Artificial Sequence
  Assay 2827 TREM27 F primer
gcggatgctggcacagc

SEQ ID NO: 20 - DNA, 19 bp, Artificial Sequence
  Assay 2827 TREM27 R primer
ggcattgcttccttctccg

SEQ ID NO: 21 - DNA, 15 bp, Artificial Sequence
  Assay 2827 FAM
cagggagcgaggtac

SEQ ID NO: 22 - DNA, 23 bp, Artificial Sequence
  SM4849 F primer
ccactcatatttcccttgtggct

SEQ ID NO: 23 - DNA, 19 bp, Artificial Sequence
  SM4849 R primer
gcaggaatagcgcctctct

SEQ ID NO: 24 - DNA, 20 bp, Artificial Sequence
  SM4849 FAM
caggatgaaggtcgattgag

SEQ ID NO: 25 - DNA, 20 bp, Artificial Sequence
  SM4849 TET
aggatgaaggtcggttgaga

SEQ ID NO: 26 - DNA, 1012 bp, Zea mays
  misc_feature (4)..(4): N = Adenine or Guanine or Cytosine or Thymine
  misc_feature (38)..(38): N = Adenine or Guanine or Cytosine or Thymine
  misc_feature (45)..(45): N = Adenine or Guanine or Cytosine or Thymine
  misc_feature (73)..(73): N = Adenine or Guanine or Cytosine or Thymine
  misc_feature (75)..(75): N = Adenine or Guanine or Cytosine or Thymine
  misc_feature (95)..(95): N = Adenine or Guanine or Cytosine or Thymine
  misc_feature (111)..(111): N = Adenine or Guanine or Cytosine or Thymine
  misc_feature (127)..(127): N = Adenine or Guanine or Cytosine or Thymine
  misc_feature (130)..(131): N = Adenine or Guanine or Cytosine or Thymine
  misc_feature (144)..(144): N = Adenine or Guanine or Cytosine or Thymine
  misc_feature (161)..(161): N = Adenine or Guanine or Cytosine or Thymine
  misc_feature (175)..(175): N = Adenine or Guanine or Cytosine or Thymine
  misc_feature (187)..(187): N = Adenine or Guanine or Cytosine or Thymine
  misc_feature (207)..(207): N = Adenine or Guanine or Cytosine or Thymine
  misc_feature (212)..(212): N = Adenine or Guanine or Cytosine or Thymine
  misc_feature (232)..(232): N = Adenine or Guanine or Cytosine or Thymine
  misc_feature (255)..(255): N = Adenine or Guanine or Cytosine or Thymine
  misc_feature (259)..(259): N = Adenine or Guanine or Cytosine or Thymine
  misc_feature (261)..(262): N = Adenine or Guanine or Cytosine or Thymine
  misc_feature (269)..(273): N = Adenine or Guanine or Cytosine or Thymine
  misc_feature (280)..(280): N = Adenine or Guanine or Cytosine or Thymine
  misc_feature (307)..(307): N = Adenine or Guanine or Cytosine or Thymine
  misc_feature (369)..(369): N = Adenine or Guanine or Cytosine or Thymine
  misc_feature (371)..(371): N = Adenine or Guanine or Cytosine or Thymine
  misc_feature (384)..(384): N = Adenine or Guanine or Cytosine or Thymine
  misc_feature (406)..(406): N = Adenine or Guanine or Cytosine or Thymine
  misc_feature (424)..(424): N = Adenine or Guanine or Cytosine or Thymine
  misc_feature (431)..(444): N = Adenine or Guanine or Cytosine or Thymine
  misc_feature (452)..(452): N = Adenine or Guanine or Cytosine or Thymine
  misc_feature (464)..(464): N = Adenine or Guanine or Cytosine or Thymine
  misc_feature (612)..(612): N = Adenine or Guanine or Cytosine or Thymine
  misc_feature (650)..(652): N = Adenine or Guanine or Cytosine or Thymine
  misc_feature (668)..(669): N = Adenine or Guanine or Cytosine or Thymine
  misc_feature (673)..(682): N = Adenine or Guanine or Cytosine or Thymine
  misc_feature (692)..(692): N = Adenine or Guanine or Cytosine or Thymine
  misc_feature (744)..(745): N = Adenine or Guanine or Cytosine or Thymine
  misc_feature (753)..(753): N = Adenine or Guanine or Cytosine or Thymine
  misc_feature (772)..(775): N = Adenine or Guanine or Cytosine or Thymine
  misc_feature (802)..(802): N = Adenine or Guanine or Cytosine or Thymine
  misc_feature (816)..(816): N = Adenine or Guanine or Cytosine or Thymine
  misc_feature (819)..(819): N = Adenine or Guanine or Cytosine or Thymine
  misc_feature (834)..(834): N = Adenine or Guanine or Cytosine or Thymine
  misc_feature (837)..(837): N = Adenine or Guanine or Cytosine or Thymine
  misc_feature (855)..(855): N = Adenine or Guanine or Cytosine or Thymine
  misc_feature (857)..(857): N = Adenine or Guanine or Cytosine or Thymine
  misc_feature (863)..(863): N = Adenine or Guanine or Cytosine or Thymine
  misc_feature (895)..(895): N = Adenine or Guanine or Cytosine or Thymine
  misc_feature (918)..(918): N = Adenine or Guanine or Cytosine or Thymine
  misc_feature (937)..(937): N = Adenine or Guanine or Cytosine or Thymine
  misc_feature (952)..(952): N = Adenine or Guanine or Cytosine or Thymine
  misc_feature (981)..(981): N = Adenine or Guanine or Cytosine or Thymine
  misc_feature (987)..(987): N = Adenine or Guanine or Cytosine or Thymine
  misc_feature (999)..(1000): N = Adenine or Guanine or Cytosine or Thymine
  misc_feature (1008)..(1008): N = Adenine or Guanine or Cytosine or Thymine
  misc_feature (1011)..(1011): N = Adenine or Guanine or Cytosine or Thymine
tctncacatagattcaaagatatttagtttggatagtnacagagnaagtcatactgatctgctggagctgtgnanctgaagagaattttagcaantgtataccagagtcanctttctcttatctaantannacaatctttcaantcctggatgctacaaanctgcaagctgtganaaattaaagatnaaatattatttagttatacntatanctggatcttggactttgaantttaaaagtaggttcatgtaatntgcnannttttttnnnnnaaaaaangaagacgcacaagttcttgtaatgtcncatgtcacaagccttttccaataaacaattccaagaacgtataaagtttcagtacttctctntngatataggcaacntgctggtgcatgggtatatatntttagttggcaacccacncatacannnnnnnnnnnnnntatgcagnggtgctttgaanccactcatatttcccttgtggctgaaacaggatgaaggtcggttgagagaagccctgaagttcgcaaacgtctgtggagctctcaccgtgacacagagaggcgctattcctgcgctgcccacccgacaacaagtgcttgatgccctgnccaattttgttgcttgaaagtagccgtgcacacctacnnnttgtgtattgtaatgnngacngcctgagcnccatgtcganagattacaggcgtgatttcgcttgcataccacttggcatttttgttggtgcnnaccaaagnccccccagccaaaccaagnnnntcggtggaagaggttgtttcccgtcgnattttgatgtgtgnagntgtgtgttggggacnttngggttaacactttgtggncntgtgtnatgtgtgtgctgatgttgtaatgctgctgggnagaacatgacacagcggagcagntttggtacctgaaaacatnagctatgaatgagtnattcagttattgggcccaatttgcgttgnttcccnagcatcaggtcnnccccctcnttnc

SEQ ID NO: 27 - DNA, 1012 bp, Zea mays
  misc_feature (4)..(4): N = Adenine or Guanine or Cytosine or Thymine
  misc_feature (38)..(38): N = Adenine or Guanine or Cytosine or Thymine
  misc_feature (45)..(45): N = Adenine or Guanine or Cytosine or Thymine
  misc_feature (73)..(73): N = Adenine or Guanine or Cytosine or Thymine
  misc_feature (75)..(75): N = Adenine or Guanine or Cytosine or Thymine
  misc_feature (95)..(95): N = Adenine or Guanine or Cytosine or Thymine
  misc_feature (111)..(111): N = Adenine or Guanine or Cytosine or Thymine
  misc_feature (127)..(127): N = Adenine or Guanine or Cytosine or Thymine
  misc_feature (130)..(131): N = Adenine or Guanine or Cytosine or Thymine
  misc_feature (144)..(144): N = Adenine or Guanine or Cytosine or Thymine
  misc_feature (161)..(161): N = Adenine or Guanine or Cytosine or Thymine
  misc_feature (175)..(175): N = Adenine or Guanine or Cytosine or Thymine
  misc_feature (187)..(187): N = Adenine or Guanine or Cytosine or Thymine
  misc_feature (207)..(207): N = Adenine or Guanine or Cytosine or Thymine
  misc_feature (212)..(212): N = Adenine or Guanine or Cytosine or Thymine
  misc_feature (232)..(232): N = Adenine or Guanine or Cytosine or Thymine
  misc_feature (255)..(255): N = Adenine or Guanine or Cytosine or Thymine
  misc_feature (259)..(259): N = Adenine or Guanine or Cytosine or Thymine
  misc_feature (261)..(262): N = Adenine or Guanine or Cytosine or Thymine
  misc_feature (269)..(273): N = Adenine or Guanine or Cytosine or Thymine
  misc_feature (280)..(280): N = Adenine or Guanine or Cytosine or Thymine
  misc_feature (307)..(307): N = Adenine or Guanine or Cytosine or Thymine
  misc_feature (369)..(369): N = Adenine or Guanine or Cytosine or Thymine
  misc_feature (371)..(371): N = Adenine or Guanine or Cytosine or Thymine
  misc_feature (384)..(384): N = Adenine or Guanine or Cytosine or Thymine
  misc_feature (406)..(406): N = Adenine or Guanine or Cytosine or Thymine
  misc_feature (424)..(424): N = Adenine or Guanine or Cytosine or Thymine
  misc_feature (431)..(444): N = Adenine or Guanine or Cytosine or Thymine
  misc_feature (452)..(464): N = Adenine or Guanine or Cytosine or Thymine
  misc_feature (612)..(612): N = Adenine or Guanine or Cytosine or Thymine
  misc_feature (650)..(652): N = Adenine or Guanine or Cytosine or Thymine
  misc_feature (668)..(669): N = Adenine or Guanine or Cytosine or Thymine
  misc_feature (673)..(673): N = Adenine or Guanine or Cytosine or Thymine
  misc_feature (682)..(682): N = Adenine or Guanine or Cytosine or Thymine
  misc_feature (692)..(692): N = Adenine or Guanine or Cytosine or Thymine
  misc_feature (744)..(745): N = Adenine or Guanine or Cytosine or Thymine
  misc_feature (753)..(753): N = Adenine or Guanine or Cytosine or Thymine
  misc_feature (772)..(775): N = Adenine or Guanine or Cytosine or Thymine
  misc_feature (802)..(802): N = Adenine or Guanine or Cytosine or Thymine
  misc_feature (816)..(816): N = Adenine or Guanine or Cytosine or Thymine
  misc_feature (819)..(819): N = Adenine or Guanine or Cytosine or Thymine
  misc_feature (834)..(834): N = Adenine or Guanine or Cytosine or Thymine
  misc_feature (837)..(837): N = Adenine or Guanine or Cytosine or Thymine
  misc_feature (855)..(855): N = Adenine or Guanine or Cytosine or Thymine
  misc_feature (857)..(857): N = Adenine or Guanine or Cytosine or Thymine
  misc_feature (863)..(863): N = Adenine or Guanine or Cytosine or Thymine
  misc_feature (895)..(895): N = Adenine or Guanine or Cytosine or Thymine
  misc_feature (918)..(918): N = Adenine or Guanine or Cytosine or Thymine
  misc_feature (937)..(937): N = Adenine or Guanine or Cytosine or Thymine
  misc_feature (952)..(952): N = Adenine or Guanine or Cytosine or Thymine
  misc_feature (981)..(981): N = Adenine or Guanine or Cytosine or Thymine
  misc_feature (987)..(987): N = Adenine or Guanine or Cytosine or Thymine
  misc_feature (999)..(1000): N = Adenine or Guanine or Cytosine or Thymine
  misc_feature (1008)..(1008): N = Adenine or Guanine or Cytosine or Thymine
  misc_feature (1011)..(1011): N = Adenine or Guanine or Cytosine or Thymine
tctncacatagattcaaagatatttagtttggatagtnacagagnaagtcatactgatctgctggagctgtgnanctgaagagaattttagcaantgtataccagagtcanctttctcttatctaantannacaatctttcaantcctggatgctacaaanctgcaagctgtganaaattaaagatnaaatattatttagttatacntatanctggatcttggactttgaantttaaaagtaggttcatgtaatntgcnannttttttnnnnnaaaaaangaagacgcacaagttcttgtaatgtcncatgtcacaagccttttccaataaacaattccaagaacgtataaagtttcagtacttctctntngatataggcaacntgctggtgcatgggtatatatntttagttggcaacccacncatacannnnnnnnnnnnnntatgcagnggtgctttgaanccactcatatttcccttgtggctgaaacaggatgaaggtcgattgagagaagccctgaagttcgcaaacgtctgtggagctctcaccgtgacacagagaggcgctattcctgcgctgcccacccgacaacaagtgcttgatgccctgnccaattttgttgcttgaaagtagccgtgcacacctacnnnttgtgtattgtaatgnngacngcctgagcnccatgtcganagattacaggcgtgatttcgcttgcataccacttggcatttttgttggtgcnnaccaaagnccccccagccaaaccaagnnnntcggtggaagaggttgtttcccgtcgnattttgatgtgtgnagntgtgtgttggggacnttngggttaacactttgtggncntgtgtnatgtgtgtgctgatgttgtaatgctgctgggnagaacatgacacagcggagcagntttggtacctgaaaacatnagctatgaatgagtnattcagttattgggcccaatttgcgttgnttcccnagcatcaggtcnnccccctcnttnc

SEQ ID NO: 28 - DNA, 20 bp, Artificial Sequence
  SM8047 F primer
gtattccagcttctgtagtt

SEQ ID NO: 29 - DNA, 17 bp, Artificial Sequence
  SM8047 R primer
ttgcacgtcatgagttc

SEQ ID NO: 30 - DNA, 17 bp, Artificial Sequence
  SM8047 FAM
ttgtgtcagataattga

SEQ ID NO: 31 - DNA, 17 bp, Artificial Sequence
  SM8047 TET
tgtgtcagattattgaa

SEQ ID NO: 32 - DNA, 1041 bp, Zea mays
  misc_feature (13)..(19): N = Adenine or Guanine or Cytosine or Thymine
  misc_feature (131)..(131): N = Adenine or Guanine or Cytosine or Thymine
  misc_feature (153)..(153): N = Adenine or Guanine or Cytosine or Thymine
  misc_feature (177)..(182): N = Adenine or Guanine or Cytosine or Thymine
  misc_feature (190)..(190): N = Adenine or Guanine or Cytosine or Thymine
  misc_feature (194)..(194): N = Adenine or Guanine or Cytosine or Thymine
  misc_feature (196)..(196): N = Adenine or Guanine or Cytosine or Thymine
  misc_feature (203)..(203): N = Adenine or Guanine or Cytosine or Thymine
  misc_feature (216)..(221): N = Adenine or Guanine or Cytosine or Thymine
  misc_feature (224)..(224): N = Adenine or Guanine or Cytosine or Thymine
  misc_feature (237)..(237): N = Adenine or Guanine or Cytosine or Thymine
  misc_feature (240)..(240): N = Adenine or Guanine or Cytosine or Thymine
  misc_feature (283)..(283): N = Adenine or Guanine or Cytosine or Thymine
  misc_feature (307)..(307): N = Adenine or Guanine or Cytosine or Thymine
  misc_feature (349)..(350): N = Adenine or Guanine or Cytosine or Thymine
  misc_feature (354)..(354): N = Adenine or Guanine or Cytosine or Thymine
  misc_feature (358)..(359): N = Adenine or Guanine or Cytosine or Thymine
  misc_feature (362)..(363): N = Adenine or Guanine or Cytosine or Thymine
  misc_feature (365)..(368): N = Adenine or Guanine or Cytosine or Thymine
  misc_feature (398)..(398): N = Adenine or Guanine or Cytosine or Thymine
  misc_feature (400)..(427): N = Adenine or Guanine or Cytosine or Thymine
  misc_feature (433)..(434): N = Adenine or Guanine or Cytosine or Thymine
  misc_feature (437)..(437): N = Adenine or Guanine or Cytosine or Thymine
  misc_feature (465)..(465): N = Adenine or Guanine or Cytosine or Thymine
  misc_feature (467)..(476): N = Adenine or Guanine or Cytosine or Thymine
  misc_feature (478)..(478): N = Adenine or Guanine or Cytosine or Thymine
  misc_feature (480)..(484): N = Adenine or Guanine or Cytosine or Thymine
  misc_feature (495)..(495): N = Adenine or Guanine or Cytosine or Thymine
  misc_feature (498)..(499): N = Adenine or Guanine or Cytosine or Thymine
  misc_feature (502)..(506): N = Adenine or Guanine or Cytosine or Thymine
  misc_feature (508)..(511): N = Adenine or Guanine or Cytosine or Thymine
  misc_feature (729)..(729): N = Adenine or Guanine or Cytosine or Thymine
  misc_feature (744)..(744): N = Adenine or Guanine or Cytosine or Thymine
  misc_feature (754)..(754): N = Adenine or Guanine or Cytosine or Thymine
  misc_feature (768)..(768): N = Adenine or Guanine or Cytosine or Thymine
  misc_feature (777)..(777): N = Adenine or Guanine or Cytosine or Thymine
  misc_feature (780)..(780): N = Adenine or Guanine or Cytosine or Thymine
  misc_feature (818)..(818): N = Adenine or Guanine or Cytosine or Thymine
  misc_feature (828)..(828): N = Adenine or Guanine or Cytosine or Thymine
  misc_feature (831)..(831): N = Adenine or Guanine or Cytosine or Thymine
  misc_feature (837)..(837): N = Adenine or Guanine or Cytosine or Thymine
  misc_feature (853)..(858): N = Adenine or Guanine or Cytosine or Thymine
  misc_feature (860)..(864): N = Adenine or Guanine or Cytosine or Thymine
  misc_feature (868)..(869): N = Adenine or Guanine or Cytosine or Thymine
  misc_feature (892)..(892): N = Adenine or Guanine or Cytosine or Thymine
  misc_feature (895)..(895): N = Adenine or Guanine or Cytosine or Thymine
  misc_feature (912)..(912): N = Adenine or Guanine or Cytosine or Thymine
  misc_feature (927)..(930): N = Adenine or Guanine or Cytosine or Thymine
  misc_feature (946)..(946): N = Adenine or Guanine or Cytosine or Thymine
  misc_feature (952)..(955): N = Adenine or Guanine or Cytosine or Thymine
tctcctccatgcnnnnnnngctgcaggctcgctacaggtgctatagggctcataagtaccacaagaagctgaaatgcgcagccattgttgcacagtgcagatggagaggaaggatcgcgaggaaagagctnaagaaactcaagatggtatgcntcatatttagagttatgtagcagnnnnnnagtgtgcnaaangnttgtcanttctaatgcgttnnnnnnatntgctgtcatcttntgntcaggaagcaagagaaacgggtgcgctcaaggaagcgaagganaaactcgaaaagaaagtggaaganctcacatggcgcgtgcagttagagaaacgactaagggtatcnntttntttnnctnncnnnnatcttctttgttccttctcatatcttctcntnnnnnnnnnnnnnnnnnnnnnnnnnnnnagttgnntcngtattccagcttctgtagttttaatatnannnnnnnnnngntncggnattggcaaatnacnntannnnntnnnntttttcaataatctgacacaaatgaactcatgacgtgcaaacagacagacctggaagaagcaaaagctcaagaggtgtcgaaactgcagaactctatggaagcattacaggctaaactggacgagacaaacacaaagctcgctaaggagcgagaagctgctaagactatcgaagaagcgcctcctgtggtgcaggaaactcaggtcctcgtccaaganaccgaaaagatcgantccttgacancagaggttcaagancttaaggtncgnctgcctacagcaactgtttgtccggaatttcctactcntttttctaanccntttttncgacagcatgcatgannnnnncnnnnncccnnatcagacaaacttttgaagtgcntcntgttttgatgattttgncacctacgcttcgtnnnngacgtcttggcttcantttttnnnnctgtagacttcattacaatcagagaaagaaagggctggcgatttggaaaagaaacactctgaagagcaacaggcgaatgaagaaaa

SEQ ID NO: 33 - DNA, 1041 bp, Zea mays
  misc_feature (13)..(19): N = Adenine or Guanine or Cytosine or Thymine
  misc_feature (131)..(131): N = Adenine or Guanine or Cytosine or Thymine
  misc_feature (153)..(153): N = Adenine or Guanine or Cytosine or Thymine
  misc_feature (177)..(182): N = Adenine or Guanine or Cytosine or Thymine
  misc_feature (190)..(190): N = Adenine or Guanine or Cytosine or Thymine
  misc_feature (194)..(194): N = Adenine or Guanine or Cytosine or Thymine
  misc_feature (196)..(196): N = Adenine or Guanine or Cytosine or Thymine
  misc_feature (203)..(203): N = Adenine or Guanine or Cytosine or Thymine
  misc_feature (216)..(221): N = Adenine or Guanine or Cytosine or Thymine
  misc_feature (224)..(224): N = Adenine or Guanine or Cytosine or Thymine
  misc_feature (237)..(237): N = Adenine or Guanine or Cytosine or Thymine
  misc_feature (240)..(240): N = Adenine or Guanine or Cytosine or Thymine
  misc_feature (283)..(283): N = Adenine or Guanine or Cytosine or Thymine
  misc_feature (307)..(307): N = Adenine or Guanine or Cytosine or Thymine
  misc_feature (349)..(350): N = Adenine or Guanine or Cytosine or Thymine
  misc_feature (354)..(354): N = Adenine or Guanine or Cytosine or Thymine
  misc_feature (358)..(359): N = Adenine or Guanine or Cytosine or Thymine
  misc_feature (362)..(363): N = Adenine or Guanine or Cytosine or Thymine
  misc_feature (365)..(368): N = Adenine or Guanine or Cytosine or Thymine
  misc_feature (398)..(398): N = Adenine or Guanine or Cytosine or Thymine
  misc_feature (400)..(427): N = Adenine or Guanine or Cytosine or Thymine
  misc_feature (433)..(434): N = Adenine or Guanine or Cytosine or Thymine
  misc_feature (437)..(437): N = Adenine or Guanine or Cytosine or Thymine
  misc_feature (465)..(465): N = Adenine or Guanine or Cytosine or Thymine
  misc_feature (467)..(476): N = Adenine or Guanine or Cytosine or Thymine
  misc_feature (478)..(478): N = Adenine or Guanine or Cytosine or Thymine
  misc_feature (480)..(480): N = Adenine or Guanine or Cytosine or Thymine
  misc_feature (484)..(484): N = Adenine or Guanine or Cytosine or Thymine
  misc_feature (495)..(495): N = Adenine or Guanine or Cytosine or Thymine
  misc_feature (498)..(499): N = Adenine or Guanine or Cytosine or Thymine
  misc_feature (502)..(506): N = Adenine or Guanine or Cytosine or Thymine
  misc_feature (508)..(511): N = Adenine or Guanine or Cytosine or Thymine
  misc_feature (729)..(729): N = Adenine or Guanine or Cytosine or Thymine
  misc_feature (744)..(744): N = Adenine or Guanine or Cytosine or Thymine
  misc_feature (754)..(754): N = Adenine or Guanine or Cytosine or Thymine
  misc_feature (768)..(768): N = Adenine or Guanine or Cytosine or Thymine
  misc_feature (777)..(777): N = Adenine or Guanine or Cytosine or Thymine
  misc_feature (780)..(780): N = Adenine or Guanine or Cytosine or Thymine
  misc_feature (818)..(818): N = Adenine or Guanine or Cytosine or Thymine
  misc_feature (828)..(828): N = Adenine or Guanine or Cytosine or Thymine
  misc_feature (831)..(831): N = Adenine or Guanine or Cytosine or Thymine
  misc_feature (837)..(837): N = Adenine or Guanine or Cytosine or Thymine
  misc_feature (853)..(858): N = Adenine or Guanine or Cytosine or Thymine
  misc_feature (860)..(864): N = Adenine or Guanine or Cytosine or Thymine
  misc_feature (868)..(869): N = Adenine or Guanine or Cytosine or Thymine
  misc_feature (892)..(892): N = Adenine or Guanine or Cytosine or Thymine
  misc_feature (895)..(895): N = Adenine or Guanine or Cytosine or Thymine
  misc_feature (912)..(912): N = Adenine or Guanine or Cytosine or Thymine
  misc_feature (927)..(930): N = Adenine or Guanine or Cytosine or Thymine
  misc_feature (946)..(946): N = Adenine or Guanine or Cytosine or Thymine
  misc_feature (952)..(955): N = Adenine or Guanine or Cytosine or Thymine
tctcctccatgcnnnnnnngctgcaggctcgctacaggtgctatagggctcataagtaccacaagaagctgaaatgcgcagccattgttgcacagtgcagatggagaggaaggatcgcgaggaaagagctnaagaaactcaagatggtatgcntcatatttagagttatgtagcagnnnnnnagtgtgcnaaangnttgtcanttctaatgcgttnnnnnnatntgctgtcatcttntgntcaggaagcaagagaaacgggtgcgctcaaggaagcgaagganaaactcgaaaagaaagtggaaganctcacatggcgcgtgcagttagagaaacgactaagggtatcnntttntttnnctnncnnnnatcttctttgttccttctcatatcttctcntnnnnnnnnnnnnnnnnnnnnnnnnnnnnagttgnntcngtattccagcttctgtagttttaatatnannnnnnnnnngntncggnattggcaaatnacnntannnnntnnnntttttcaattatctgacacaaatgaactcatgacgtgcaaacagacagacctggaagaagcaaaagctcaagaggtgtcgaaactgcagaactctatggaagcattacaggctaaactggacgagacaaacacaaagctcgctaaggagcgagaagctgctaagactatcgaagaagcgcctcctgtggtgcaggaaactcaggtcctcgtccaaganaccgaaaagatcgantccttgacancagaggttcaagancttaaggtncgnctgcctacagcaactgtttgtccggaatttcctactcntttttctaanccntttttncgacagcatgcatgannnnnncnnnnncccnnatcagacaaacttttgaagtgcntcntgttttgatgattttgncacctacgcttcgtnnnngacgtcttggcttcantttttnnnnctgtagacttcattacaatcagagaaagaaagggctggcgatttggaaaagaaacactctgaagagcaacaggcgaatgaagaaaa

SEQ ID NO: 34 - DNA, 27 bp, Artificial Sequence
  SM8133 F primer
agcaactgtaaaaacttggatgtgatg

SEQ ID NO: 35 - DNA, 26 bp, Artificial Sequence
  SM8133 R primer
gcagcttacgttaggatagattgagc

SEQ ID NO: 36 - DNA, 20 bp, Artificial Sequence
  SM8133 FAM
ccgatttgaaatttcttcag

SEQ ID NO: 37 - DNA, 20 bp, Artificial Sequence
  SM8133 TET
ccgatttgaaatttcttgag

SEQ ID NO: 38 - DNA, 1003 bp, Zea mays
  misc_feature (94)..(94): N = Adenine or Guanine or Cytosine or Thymine
  misc_feature (130)..(130): N = Adenine or Guanine or Cytosine or Thymine
  misc_feature (176)..(176): N = Adenine or Guanine or Cytosine or Thymine
  misc_feature (183)..(183): N = Adenine or Guanine or Cytosine or Thymine
  misc_feature (234)..(234): N = Adenine or Guanine or Cytosine or Thymine
  misc_feature (345)..(345): N = Adenine or Guanine or Cytosine or Thymine
  misc_feature (404)..(404): N = Adenine or Guanine or Cytosine or Thymine
  misc_feature (407)..(407): N = Adenine or Guanine or Cytosine or Thymine
  misc_feature (430)..(430): N = Adenine or Guanine or Cytosine or Thymine
  misc_feature (473)..(473): N = Adenine or Guanine or Cytosine or Thymine
  misc_feature (483)..(483): N = Adenine or Guanine or Cytosine or Thymine
  misc_feature (504)..(504): N = Adenine or Guanine or Cytosine or Thymine
  misc_feature (508)..(508): N = Adenine or Guanine or Cytosine or Thymine
  misc_feature (525)..(530): N = Adenine or Guanine or Cytosine or Thymine
  misc_feature (569)..(569): N = Adenine or Guanine or Cytosine or Thymine
  misc_feature (571)..(571): N = Adenine or Guanine or Cytosine or Thymine
  misc_feature (585)..(585): N = Adenine or Guanine or Cytosine or Thymine
  misc_feature (587)..(587): N = Adenine or Guanine or Cytosine or Thymine
  misc_feature (619)..(619): N = Adenine or Guanine or Cytosine or Thymine
  misc_feature (623)..(624): N = Adenine or Guanine or Cytosine or Thymine
  misc_feature (638)..(638): N = Adenine or Guanine or Cytosine or Thymine
  misc_feature (665)..(665): N = Adenine or Guanine or Cytosine or Thymine
  misc_feature (688)..(688): N = Adenine or Guanine or Cytosine or Thymine
  misc_feature (709)..(710): N = Adenine or Guanine or Cytosine or Thymine
  misc_feature (741)..(741): N = Adenine or Guanine or Cytosine or Thymine
  misc_feature (795)..(795): N = Adenine or Guanine or Cytosine or Thymine
  misc_feature (839)..(840): N = Adenine or Guanine or Cytosine or Thymine
  misc_feature (851)..(851): N = Adenine or Guanine or Cytosine or Thymine
  misc_feature (879)..(879): N = Adenine or Guanine or Cytosine or Thymine
  misc_feature (940)..(940): N = Adenine or Guanine or Cytosine or Thymine
  misc_feature (966)..(966): N = Adenine or Guanine or Cytosine or Thymine
tccacttcaccgacagcttccgcgccccggacgggaaggtgtactacggcttcgtcacgccgcggggcctgtcgctgttcaggaccgggctcgncgtcgaggtgcccagggaggaaaggtaccggctcgncttcgtcgacgtcgtgcacgctgtcatgtccgtgctggtctttgcngccgtcncgctcgccgactaccgggtctccgggtgcctcgtcgccggccaccgcaagnagatggacgaggtgatggagagcttcccgctcatggtgggcgccgtgtgcagcggcctcttcctcttgttccccaacacccgctacggcatcggttgtttggctccgtaanaaacagcagactggaacagagagtacggcagtgtaactttcttccgtacctgtgaatcnggnttgatcattttatgcttcatgtnttcttagcaactgtaaaaacttggatgtgatgtgatcctatcnttaatcagtnccgatttgaaatttcttgagnatgnattatacaagagaatgnnnngncaccaaaaatagctttacatcagatgcaaaatgcattcntntcaaaagaatggtnangactggctcaatctatcctaacgtaagctgcngccnntgtatcctacattntggcaagatactagtattttacaagcnacacagtaagcaaagcagcactntcctacctacccaaaaaaaanngatgtgacctgcattctgcctacaatgcatntcccctagtttgactagaaactcttcaaactgggacgaaacaaacttaagatanccaaaacttcgcttgccatccagttctgcaagattcctcttagnnggggctgtatntgtcaaatgtcaagaacgttttagcgtnacggtgtctgttcaagcaacggccaacagcaaccaccgtaaaataaagcatcatttgcatnatctctgttattctttgcaacatgtncctcgttttgttttctgaaacttatcccagaggcaca

SEQ ID NO: 39 - DNA, 1003 bp, Zea mays
  misc_feature (94)..(94): N = Adenine or Guanine or Cytosine or Thymine
  misc_feature (130)..(130): N = Adenine or Guanine or Cytosine or Thymine
  misc_feature (176)..(176): N = Adenine or Guanine or Cytosine or Thymine
  misc_feature (183)..(183): N = Adenine or Guanine or Cytosine or Thymine
  misc_feature (234)..(234): N = Adenine or Guanine or Cytosine or Thymine
  misc_feature (345)..(345): N = Adenine or Guanine or Cytosine or Thymine
  misc_feature (404)..(404): N = Adenine or Guanine or Cytosine or Thymine
  misc_feature (407)..(407): N = Adenine or Guanine or Cytosine or Thymine
  misc_feature (430)..(430): N = Adenine or Guanine or Cytosine or Thymine
  misc_feature (473)..(473): N = Adenine or Guanine or Cytosine or Thymine
  misc_feature (483)..(483): N = Adenine or Guanine or Cytosine or Thymine
  misc_feature (504)..(504): N = Adenine or Guanine or Cytosine or Thymine
  misc_feature (508)..(508): N = Adenine or Guanine or Cytosine or Thymine
  misc_feature (525)..(530): N = Adenine or Guanine or Cytosine or Thymine
  misc_feature (569)..(569): N = Adenine or Guanine or Cytosine or Thymine
  misc_feature (571)..(571): N = Adenine or Guanine or Cytosine or Thymine
  misc_feature (585)..(585): N = Adenine or Guanine or Cytosine or Thymine
  misc_feature (587)..(587): N = Adenine or Guanine or Cytosine or Thymine
  misc_feature (619)..(619): N = Adenine or Guanine or Cytosine or Thymine
  misc_feature (623)..(624): N = Adenine or Guanine or Cytosine or Thymine
  misc_feature (638)..(638): N = Adenine or Guanine or Cytosine or Thymine
  misc_feature (665)..(665): N = Adenine or Guanine or Cytosine or Thymine
  misc_feature (688)..(688): N = Adenine or Guanine or Cytosine or Thymine
  misc_feature (709)..(710): N = Adenine or Guanine or Cytosine or Thymine
  misc_feature (741)..(741): N = Adenine or Guanine or Cytosine or Thymine
  misc_feature (795)..(795): N = Adenine or Guanine or Cytosine or Thymine
  misc_feature (839)..(840): N = Adenine or Guanine or Cytosine or Thymine
  misc_feature (851)..(851): N = Adenine or Guanine or Cytosine or Thymine
  misc_feature (879)..(879): N = Adenine or Guanine or Cytosine or Thymine
  misc_feature (940)..(940): N = Adenine or Guanine or Cytosine or Thymine
  misc_feature (966)..(966): N = Adenine or Guanine or Cytosine or Thymine
tccacttcaccgacagcttccgcgccccggacgggaaggtgtactacggcttcgtcacgccgcggggcctgtcgctgttcaggaccgggctcgncgtcgaggtgcccagggaggaaaggtaccggctcgncttcgtcgacgtcgtgcacgctgtcatgtccgtgctggtctttgcngccgtcncgctcgccgactaccgggtctccgggtgcctcgtcgccggccaccgcaagnagatggacgaggtgatggagagcttcccgctcatggtgggcgccgtgtgcagcggcctcttcctcttgttccccaacacccgctacggcatcggttgtttggctccgtaanaaacagcagactggaacagagagtacggcagtgtaactttcttccgtacctgtgaatcnggnttgatcattttatgcttcatgtnttcttagcaactgtaaaaacttggatgtgatgtgatcctatcnttaatcagtnccgatttgaaatttcttcagnatgnattatacaagagaatgnnnngncaccaaaaatagctttacatcagatgcaaaatgcattcntntcaaaagaatggtnangactggctcaatctatcctaacgtaagctgcngccnntgtatcctacattntggcaagatactagtattttacaagcnacacagtaagcaaagcagcactntcctacctacccaaaaaaaanngatgtgacctgcattctgcctacaatgcatntcccctagtttgactagaaactcttcaaactgggacgaaacaaacttaagatanccaaaacttcgcttgccatccagttctgcaagattcctcttagnnggggctgtatntgtcaaatgtcaagaacgttttagcgtnacggtgtctgttcaagcaacggccaacagcaaccaccgtaaaataaagcatcatttgcatnatctctgttattctttgcaacatgtncctcgttttgttttctgaaacttatcccagaggcaca

SEQ ID NO: 40 - DNA, 17 bp, Artificial Sequence
  SM8029 F primer
tcacgtgccaccggaaa

SEQ ID NO: 41 - DNA, 25 bp, Artificial Sequence
  SM8029 R primer
ccacatgccaccgaaaataaattgt

SEQ ID NO: 42 - DNA, 17 bp, Artificial Sequence
  SM8029 FAM
ttagcggcaaacgactt

SEQ ID NO: 43 - DNA, 19 bp, Artificial Sequence
  SM8029 TET
tttagcggcaaatgactta

SEQ ID NO: 44 - DNA, 1003 bp, Zea mays
  misc_feature (230)..(231): N = Adenine or Guanine or Cytosine or Thymine
ctttgcttaataacatctagagtgctgcatgcgtttttcacattttatactaaagtatttatatatgtaaagtattttaaatgttaaagtctgcatggcacggtaaatgaccgacaatttagggattgatgttcttgatgagcttacgaatagcttttaggtcgtttgatttgccgaaatgtaatgcacacggtttatatgggataatggatgccaatactttttttttnnggttgagctctttctagtagtaccatctaattcaattataactagtaattattaccgtgataagagatcccattaacaacgtatgaatcaaacaacacctttagttccaaagattaaatatcaatgcctaacgagtcctaaaaatgaccactaccgcaaaaatggagcgtccttccaaaaaacatttgtttttgttatcttcaaccactgttgatataagcttcagcaccgactaaaattttcgatggtcacgtgccaccggaaataagtcgtttgccgctaaaaataagcaattttcaacaggaaacaatttattttcggtggcatgtggccgccaaaaatatttggccgaaaataagatcttatttttggcggcccaccctatactgtcaaaaaaaattctacggccttcagtggccaacgaaaataagaaaaaaaactttttaccgccacacgtgtggccgccaaaaataaatatggtttcttttcggtggccccctagccgcaaaaataaacagttttagaaaaatatacagaaagaaaattacaccagattcaaccacgtaacaaatatatacacatatacatcagattgtaacaaatatatacacatatacatcagattgacaacgcaagtcatctcatcaaattcacaacacatgataaaatatatcatattaaaccacacaagtcatcacatcgaccaaattcgcaacaccaatatcacaagcttctcaattctacacataataaaatacacatgcctcgagcc

SEQ ID NO: 45 - DNA, 1003 bp, Zea mays
  misc_feature (230)..(231): N = Adenine or Guanine or Cytosine or Thymine
ctttgcttaataacatctagagtgctgcatgcgtttttcacattttatactaaagtatttatatatgtaaagtattttaaatgttaaagtctgcatggcacggtaaatgaccgacaatttagggattgatgttcttgatgagcttacgaatagcttttaggtcgtttgatttgccgaaatgtaatgcacacggtttatatgggataatggatgccaatactttttttttnnggttgagctctttctagtagtaccatctaattcaattataactagtaattattaccgtgataagagatcccattaacaacgtatgaatcaaacaacacctttagttccaaagattaaatatcaatgcctaacgagtcctaaaaatgaccactaccgcaaaaatggagcgtccttccaaaaaacatttgtttttgttatcttcaaccactgttgatataagcttcagcaccgactaaaattttcgatggtcacgtgccaccggaaataagtcatttgccgctaaaaataagcaattttcaacaggaaacaatttattttcggtggcatgtggccgccaaaaatatttggccgaaaataagatcttatttttggcggcccaccctatactgtcaaaaaaaattctacggccttcagtggccaacgaaaataagaaaaaaaactttttaccgccacacgtgtggccgccaaaaataaatatggtttcttttcggtggccccctagccgcaaaaataaacagttttagaaaaatatacagaaagaaaattacaccagattcaaccacgtaacaaatatatacacatatacatcagattgtaacaaatatatacacatatacatcagattgacaacgcaagtcatctcatcaaattcacaacacatgataaaatatatcatattaaaccacacaagtcatcacatcgaccaaattcgcaacaccaatatcacaagcttctcaattctacacataataaaatacacatgcctcgagcc

SEQ ID NO: 46 - DNA, 24 bp, Artificial Sequence
  SM4257 F primer
cgagactcaagaacctgataggaa

SEQ ID NO: 47 - DNA, 19 bp, Artificial Sequence
  SM4257 R primer
gcagtgttggcccacgatt

SEQ ID NO: 48 - DNA, 18 bp, Artificial Sequence
  SM4257 FAM
aagtgattacctgatcgc

SEQ ID NO: 49 - DNA, 24 bp, Artificial Sequence
  SM4257 TET
accaaaataagtgattagctgatc

SEQ ID NO: 50 - DNA, 1001 bp, Zea mays
  misc_feature (192)..(192): N = Adenine or Guanine or Cytosine or Thymine
  misc_feature (213)..(213): N = Adenine or Guanine or Cytosine or Thymine
  misc_feature (240)..(240): N = Adenine or Guanine or Cytosine or Thymine
  misc_feature (282)..(282): N = Adenine or Guanine or Cytosine or Thymine
  misc_feature (291)..(291): N = Adenine or Guanine or Cytosine or Thymine
  misc_feature (297)..(297): N = Adenine or Guanine or Cytosine or Thymine
  misc_feature (387)..(387): N = Adenine or Guanine or Cytosine or Thymine
  misc_feature (416)..(416): N = Adenine or Guanine or Cytosine or Thymine
  misc_feature (435)..(435): N = Adenine or Guanine or Cytosine or Thymine
  misc_feature (601)..(601): N = Adenine or Guanine or Cytosine or Thymine
  misc_feature (613)..(613): N = Adenine or Guanine or Cytosine or Thymine
  misc_feature (710)..(710): N = Adenine or Guanine or Cytosine or Thymine
  misc_feature (977)..(977): N = Adenine or Guanine or Cytosine or Thymine
cttagtgacatgactagcaatgcaggcttgcggcatcagcctgaagagaaaaaaaaacccaacgactctcttacctgtgcagtggccgggaggagtttgcctgatggatcccttagagctgctgaatcgccaatcgcaaatgtccggggatggccttttacttgaagggtttcctctgtctccacctgtccncgaccattcagaggaataacntagggagcatcaggaggctgtaaccgnggaatctgagatgttgaacccactgtccacagcaccaaatcngcatcgagngtctgncctttgaggcctctttcagcaggttgaaggtccaaaattagtttcctatgatcaccgtcaacttcagagtatgtaactgtgctacttgantcatcagatgtaggagcctctttgatgcngctcacaaaatatcccaanaaaagctgaatattccgagactcaagaacctgataggaaagtagtggaaccaaaataagtgattacctgatcgctcagaaaatataatttcctcaactgaagtacagatattaaaacaatataatcgtgggccaacactgctacgtaaaatagtaaaggaatagcntgacttctccangttgtattgcatgggaatacatgatccttgaacaacccaatggaaatgaattgtttggtgacatgttacaaaaggtaaaggggtttgcatgataaanatagcaacattgcactaaagctacaacccttcaattggatcagaattcagacatgagatgaccccagagcatccaaatggaatatgcaatcaattacaattttaatataaagctgaccagaatagttcattatatatgaatagaaccttttagcttcttaccttcagagcagcgtcacgatttccttgtggagcagtgggacatacggttgtttgaacatttattgcctttacagtcccagtgttttttaatctctcagatatagtngcagctaattcaacaccggaatag

SEQ ID NO: 51 - DNA, 1001 bp, Zea mays
  misc_feature (192)..(192): N = Adenine or Guanine or Cytosine or Thymine
  misc_feature (213)..(213): N = Adenine or Guanine or Cytosine or Thymine
  misc_feature (240)..(240): N = Adenine or Guanine or Cytosine or Thymine
  misc_feature (282)..(282): N = Adenine or Guanine or Cytosine or Thymine
  misc_feature (291)..(291): N = Adenine or Guanine or Cytosine or Thymine
  misc_feature (297)..(297): N = Adenine or Guanine or Cytosine or Thymine
  misc_feature (387)..(387): N = Adenine or Guanine or Cytosine or Thymine
  misc_feature (416)..(416): N = Adenine or Guanine or Cytosine or Thymine
  misc_feature (435)..(435): N = Adenine or Guanine or Cytosine or Thymine
  misc_feature (601)..(601): N = Adenine or Guanine or Cytosine or Thymine
  misc_feature (613)..(613): N = Adenine or Guanine or Cytosine or Thymine
  misc_feature (710)..(710): N = Adenine or Guanine or Cytosine or Thymine
  misc_feature (977)..(977): N = Adenine or Guanine or Cytosine or Thymine
cttagtgacatgactagcaatgcaggcttgcggcatcagcctgaagagaaaaaaaaacccaacgactctcttacctgtgcagtggccgggaggagtttgcctgatggatcccttagagctgctgaatcgccaatcgcaaatgtccggggatggccttttacttgaagggtttcctctgtctccacctgtccncgaccattcagaggaataacntagggagcatcaggaggctgtaaccgnggaatctgagatgttgaacccactgtccacagcaccaaatcngcatcgagngtctgncctttgaggcctctttcagcaggttgaaggtccaaaattagtttcctatgatcaccgtcaacttcagagtatgtaactgtgctacttgantcatcagatgtaggagcctctttgatgcngctcacaaaatatcccaanaaaagctgaatattccgagactcaagaacctgataggaaagtagtggaaccaaaataagtgattagctgatcgctcagaaaatataatttcctcaactgaagtacagatattaaaacaatataatcgtgggccaacactgctacgtaaaatagtaaaggaatagcntgacttctccangttgtattgcatgggaatacatgatccttgaacaacccaatggaaatgaattgtttggtgacatgttacaaaaggtaaaggggtttgcatgataaanatagcaacattgcactaaagctacaacccttcaattggatcagaattcagacatgagatgaccccagagcatccaaatggaatatgcaatcaattacaattttaatataaagctgaccagaatagttcattatatatgaatagaaccttttagcttcttaccttcagagcagcgtcacgatttccttgtggagcagtgggacatacggttgtttgaacatttattgcctttacagtcccagtgttttttaatctctcagatatagtngcagctaattcaacaccggaatag

SEQ ID NO: 52 - DNA, 28 bp, Artificial Sequence
  SM0956BQ F primer
ggaatggagttttacttgtgctgaaatc

SEQ ID NO: 53 - DNA, 30 bp, Artificial Sequence
  SM0956BQ R primer
gattcactacctcaatcttacatgttacca

SEQ ID NO: 54 - DNA, 20 bp, Artificial Sequence
  SM0956BQ FAM
cttagttaagatcaatttag

SEQ ID NO: 55 - DNA, 22 bp, Artificial Sequence
  SM0956BQ TET
aacttagttaagatcgatttag

SEQ ID NO: 56 - DNA, 271 bp, Zea mays
aatgatagaactagatgtacttggtcgcatacaaggtacaaagacacaacaaagcttactcaagtacagtcaaagtaaattacagattcactacctcaatcttacatgttaccaaatacaaacttagttaagatcaatttagtgattgaaactgtgatcctgaaacatatcagttgtgtttgtttttgtaaggatttcagcacaagtaaaactccattccaaagaccttgtcaattgccacaactttcttacagatatagtccagtgtcct

SEQ ID NO: 57 - DNA, 271 bp, Zea mays
aatgatagaactagatgtacttggtcgcatacaaggtacaaagacacaacaaagcttactcaagtacagtcaaagtaaattacagattcactacctcaatcttacatgttaccaaatacaaacttagttaagatcgatttagtgattgaaactgtgatcctgaaacatatcagttgtgtttgtttttgtaaggatttcagcacaagtaaaactccattccaaagaccttgtcaattgccacaactttcttacagatatagtccagtgtcct

SEQ ID NO: 58 - DNA, 22 bp, Artificial Sequence
  SM0954BQ F primer
cagtgcgcctcatacagttgta

SEQ ID NO: 59 - DNA, 18 bp, Artificial Sequence
  SM0954BQ R primer
accaagccggcaaaggat

SEQ ID NO: 60 - DNA, 16 bp, Artificial Sequence
  SM0954BQ FAM
catgtgcctaggagag

SEQ ID NO: 61 - DNA, 16 bp, Artificial Sequence
  SM0954BQ TET
catgtgcctcggagag

SEQ ID NO: 62 - DNA, 182 bp, Zea mays
caaaghgaacaaataatmtcgatkcgagaaarggaaaaatggatcaarattttagagagaagaacccaggatattawtaaggaggctgatgagctaaacaagaagttggcgttrataccaagccggcaaaggatgcatgtgcctaggagagtacaactgtatgaggcgcactgtgttgaggc

SEQ ID NO: 63 - DNA, 182 bp, Zea mays
caaaghgaacaaataatmtcgatkcgagaaarggaaaaatggatcaarattttagagagaagaacccaggatattawtaaggaggctgatgagctaaacaagaagttggcgttrataccaagccggcaaaggatgcatgtgcctcggagagtacaactgtatgaggcgcactgtgttgaggc

SEQ ID NO: 64 - DNA, 21 bp, Artificial Sequence
  SM0954HQ F primer
gagctgattaaatggatcatc

SEQ ID NO: 65 - DNA, 17 bp, Artificial Sequence
  SM0954HQ R primer
gtgctagccatccattc

SEQ ID NO: 66 - DNA, 19 bp, Artificial Sequence
  SM0954HQ FAM
ttcacaaaggaactttata

SEQ ID NO: 67 - DNA, 18 bp, Artificial Sequence
  SM0954HQ TET
aggaactttgtaaaggca

SEQ ID NO: 68 - DNA, 106 bp, Zea mays
gagctgattaaatggatcatctgtttttcttcttgggtgaattcacaaaggaactttataaaggcactgaatggatggctagcactctgtcttaactatgagcctg

SEQ ID NO: 69 - DNA, 106 bp, Zea mays
gagctgattaaatggatcatctgtttttcttcttgggtgaattcacaaaggaactttgtaaaggcactgaatggatggctagcactctgtcttaactatgagcctg

SEQ ID NO: 70 - DNA, 22 bp, Artificial Sequence
  SM6568 F primer
caagtcactgcttccgtccatt

SEQ ID NO: 71 - DNA, 22 bp, Artificial Sequence
  SM6568 R primer
ggatcgacgctttgttcacctg

SEQ ID NO: 72 - DNA, 18 bp, Artificial Sequence
  SM6568 FAM
cacgacgaaaggattaaa

SEQ ID NO: 73 - DNA, 18 bp, Artificial Sequence
  SM6568 TET
cacgacgaaaggatttaa

SEQ ID NO: 74 - DNA, 1004 bp, Zea mays
  misc_feature (11)..(11): N = Adenine or Guanine or Cytosine or Thymine
  misc_feature (26)..(26): N = Adenine or Guanine or Cytosine or Thymine
  misc_feature (28)..(33): N = Adenine or Guanine or Cytosine or Thymine
  misc_feature (36)..(36): N = Adenine or Guanine or Cytosine or Thymine
  misc_feature (42)..(42): N = Adenine or Guanine or Cytosine or Thymine
  misc_feature (51)..(51): N = Adenine or Guanine or Cytosine or Thymine
  misc_feature (54)..(54): N = Adenine or Guanine or Cytosine or Thymine
  misc_feature (72)..(72): N = Adenine or Guanine or Cytosine or Thymine
  misc_feature (186)..(186): N = Adenine or Guanine or Cytosine or Thymine
  misc_feature (219)..(219): N = Adenine or Guanine or Cytosine or Thymine
  misc_feature (237)..(237): N = Adenine or Guanine or Cytosine or Thymine
  misc_feature (245)..(247): N = Adenine or Guanine or Cytosine or Thymine
  misc_feature (249)..(249): N = Adenine or Guanine or Cytosine or Thymine
  misc_feature (265)..(265): N = Adenine or Guanine or Cytosine or Thymine
  misc_feature (360)..(365): N = Adenine or Guanine or Cytosine or Thymine
  misc_feature (368)..(368): N = Adenine or Guanine or Cytosine or Thymine
  misc_feature (459)..(459): N = Adenine or Guanine or Cytosine or Thymine
  misc_feature (479)..(479): N = Adenine or Guanine or Cytosine or Thymine
  misc_feature (489)..(489): N = Adenine or Guanine or Cytosine or Thymine
  misc_feature (496)..(496): N = Adenine or Guanine or Cytosine or Thymine
  misc_feature (544)..(545): N = Adenine or Guanine or Cytosine or Thymine
  misc_feature (561)..(561): N = Adenine or Guanine or Cytosine or Thymine
  misc_feature (597)..(597): N = Adenine or Guanine or Cytosine or Thymine
  misc_feature (622)..(622): N = Adenine or Guanine or Cytosine or Thymine
  misc_feature (712)..(714): N = Adenine or Guanine or Cytosine or Thymine
  misc_feature (735)..(735): N = Adenine or Guanine or Cytosine or Thymine
  misc_feature (806)..(806): N = Adenine or Guanine or Cytosine or Thymine
  misc_feature (820)..(820): N = Adenine or Guanine or Cytosine or Thymine
  misc_feature (922)..(922): N = Adenine or Guanine or Cytosine or Thymine
  misc_feature (998)..(1001): N = Adenine or Guanine or Cytosine or Thymine
gcctctgcgangaaatggacctgcangnnnnnnccnctaccnctagagganggntggaccgtggacgcgtcnaatttcgaggtcccctgctcttccccgcagccagcgccgcctccggtggacagggctaccgctaacgtcgccgccgacgcctcaagggcacccgtctacggctctcgcgcgacnagtttcatggcttggacgaggtcctcgcagcantcgtcgtgctccgacgangcggcgcnnncngcagtagtgccggccntcgaggagccgcagagattgctgaagaaagtggtggccggcggcggtgcttgggagagctgtggcggcgcgacgggagcagcacaggaaatgagnnnnnntgncaccaagaaccacgtcatgtcggagcgaaagcgacgagagaagctcaacgagatgttcctcgtcctcaagtcactgcttccgtccattcanagggtaatgaacaagatacntaccatcganttttcanttttttaaatcctttcgtcgtgttgatttgaaaacttaattggagacnnattttttcccccaatntggcaggtgaacaaagcgtcgatcctcgccgaaacnatagcctacctcaaggagcttcagngaagggtgcaagagctggagtccagtagggaacctgcgtcgcgcccatccgaaacgacgacaaggctaataacaaggccctcccgtggcnnnaatgagagtgtgaggaaggangtctgcgcgggctccaagaggaagagcccagagctcggcagagacgacgtggagcgccccccggtcctcancatggacgccggcnccagcaacgtcaccgtcaccgtctcggacaaggacgtgctcctggaggtgcagtgccggtgggaggagctcctgatgacgcgagtgttcgacgccatcaagngcctccatttggacgtcctctcggttcaggcttcagcgccagatggcttcatggggcttaagatacgagctcaggnnnntat

SEQ ID NO: 75 - DNA, 1004 bp, Zea mays
  misc_feature (11)..(11): N = Adenine or Guanine or Cytosine or Thymine
  misc_feature (26)..(26): N = Adenine or Guanine or Cytosine or Thymine
  misc_feature (28)..(33): N = Adenine or Guanine or Cytosine or Thymine
  misc_feature (36)..(36): N = Adenine or Guanine or Cytosine or Thymine
  misc_feature (42)..(42): N = Adenine or Guanine or Cytosine or Thymine
  misc_feature (51)..(51): N = Adenine or Guanine or Cytosine or Thymine
  misc_feature (54)..(54): N = Adenine or Guanine or Cytosine or Thymine
  misc_feature (72)..(72): N = Adenine or Guanine or Cytosine or Thymine
  misc_feature (186)..(186): N = Adenine or Guanine or Cytosine or Thymine
  misc_feature (219)..(219): N = Adenine or Guanine or Cytosine or Thymine
  misc_feature (237)..(237): N = Adenine or Guanine or Cytosine or Thymine
  misc_feature (245)..(247): N = Adenine or Guanine or Cytosine or Thymine
  misc_feature (249)..(265): N = Adenine or Guanine or Cytosine or Thymine
  misc_feature (360)..(368): N = Adenine or Guanine or Cytosine or Thymine
  misc_feature (459)..(459): N = Adenine or Guanine or Cytosine or Thymine
  misc_feature (479)..(479): N = Adenine or Guanine or Cytosine or Thymine
  misc_feature (489)..(489): N = Adenine or Guanine or Cytosine or Thymine
  misc_feature (496)..(496): N = Adenine or Guanine or Cytosine or Thymine
  misc_feature (544)..(545): N = Adenine or Guanine or Cytosine or Thymine
  misc_feature (561)..(561): N = Adenine or Guanine or Cytosine or Thymine
  misc_feature (597)..(597): N = Adenine or Guanine or Cytosine or Thymine
  misc_feature (622)..(622): N = Adenine or Guanine or Cytosine or Thymine
  misc_feature (712)..(714): N = Adenine or Guanine or Cytosine or Thymine
  misc_feature (735)..(735): N = Adenine or Guanine or Cytosine or Thymine
  misc_feature (806)..(806): N = Adenine or Guanine or Cytosine or Thymine
  misc_feature (820)..(820): N = Adenine or Guanine or Cytosine or Thymine
  misc_feature (922)..(922): N = Adenine or Guanine or Cytosine or Thymine
  misc_feature (998)..(1001): N = Adenine or Guanine or Cytosine or Thymine
gcctctgcgangaaatggacctgcangnnnnnnccnctaccnctagagganggntggaccgtggacgcgtcnaatttcgaggtcccctgctcttccccgcagccagcgccgcctccggtggacagggctaccgctaacgtcgccgccgacgcctcaagggcacccgtctacggctctcgcgcgacnagtttcatggcttggacgaggtcctcgcagcantcgtcgtgctccgacgangcggcgcnnncngcagtagtgccggccntcgaggagccgcagagattgctgaagaaagtggtggccggcggcggtgcttgggagagctgtggcggcgcgacgggagcagcacaggaaatgagnnnnnntgncaccaagaaccacgtcatgtcggagcgaaagcgacgagagaagctcaacgagatgttcctcgtcctcaagtcactgcttccgtccattcanagggtaatgaacaagatacntaccatcganttttcantttttttaatcctttcgtcgtgttgatttgaaaacttaattggagacnnattttttcccccaatntggcaggtgaacaaagcgtcgatcctcgccgaaacnatagcctacctcaaggagcttcagngaagggtgcaagagctggagtccagtagggaacctgcgtcgcgcccatccgaaacgacgacaaggctaataacaaggccctcccgtggcnnnaatgagagtgtgaggaaggangtctgcgcgggctccaagaggaagagcccagagctcggcagagacgacgtggagcgccccccggtcctcancatggacgccggcnccagcaacgtcaccgtcaccgtctcggacaaggacgtgctcctggaggtgcagtgccggtgggaggagctcctgatgacgcgagtgttcgacgccatcaagngcctccatttggacgtcctctcggttcaggcttcagcgccagatggcttcatggggcttaagatacgagctcaggnnnntat

SEQ ID NO: 76 - DNA, 25 bp, Artificial Sequence
  SM0953BQ F primer
ggcttatagcttagaggcacttgaa

SEQ ID NO: 77 - DNA, 18 bp, Artificial Sequence
  SM0953BQ R primer
cctctgcaccgccatcaa

SEQ ID NO: 78 - DNA, 14 bp, Artificial Sequence
  SM0953BQ FAM
tcagcctcatcctc

SEQ ID NO: 79 - DNA, 15 bp, Artificial Sequence
  SM0953BQ TET
ctcagccttatcctc

SEQ ID NO: 80 - DNA, 288 bp, Zea mays
acgtagcacatgataagaaggctgcrttgagacagtaagacgaagaatggcargcagaagagcacgtcrgcatgctccccgcggcttatagcttagaggcacttgaatccggtgggcackctcttgccgcagtggttgaggatgaggctgaggtcgacsggyaggttgagcttgattcckaggacctctcccttgatggcggtgcagaggcacacggcggcctcgaggtcgacgagtcccttcagcagcrggcagcatggctccgtgggcggcacgcccaccttggcc

SEQ ID NO: 81 - DNA, 288 bp, Zea mays
acgtagcacatgataagaaggctgcrttgagacagtaagacgaagaatggcargcagaagagcacgtcrgcatgctccccgcggcttatagcttagaggcacttgaatccggtgggcackctcttgccgcagtggttgaggataaggctgaggtcgacsggyaggttgagcttgattcckaggacctctcccttgatggcggtgcagaggcacacggcggcctcgaggtcgacgagtcccttcagcagcrggcagcatggctccgtgggcggcacgcccaccttggcc

SEQ ID NO: 82 - DNA, 19 bp, Artificial Sequence
  SM6604 F primer
tgatcatccagggcacgac

SEQ ID NO: 83 - DNA, 22 bp, Artificial Sequence
  SM6604 R primer
gggcttaagatacgagctcagg

SEQ ID NO: 84 - DNA, 19 bp, Artificial Sequence
  SM6604 FAM
cagcaaactgcatagaaat

SEQ ID NO: 85 - DNA, 18 bp, Artificial Sequence
  SM6604 TET
cagcaaactgcataggaa

SEQ ID NO: 86 - DNA, 1004 bp, Zea mays
  misc_feature (40)..(40): N = Adenine or Guanine or Cytosine or Thymine
  misc_feature (42)..(42): N = Adenine or Guanine or Cytosine or Thymine
  misc_feature (79)..(79): N = Adenine or Guanine or Cytosine or Thymine
  misc_feature (81)..(81): N = Adenine or Guanine or Cytosine or Thymine
  misc_feature (113)..(113): N = Adenine or Guanine or Cytosine or Thymine
  misc_feature (144)..(147): N = Adenine or Guanine or Cytosine or Thymine
  misc_feature (149)..(152): N = Adenine or Guanine or Cytosine or Thymine
  misc_feature (161)..(161): N = Adenine or Guanine or Cytosine or Thymine
  misc_feature (178)..(178): N = Adenine or Guanine or Cytosine or Thymine
  misc_feature (182)..(182): N = Adenine or Guanine or Cytosine or Thymine
  misc_feature (196)..(196): N = Adenine or Guanine or Cytosine or Thymine
  misc_feature (202)..(202): N = Adenine or Guanine or Cytosine or Thymine
  misc_feature (246)..(246): N = Adenine or Guanine or Cytosine or Thymine
  misc_feature (252)..(252): N = Adenine or Guanine or Cytosine or Thymine
  misc_feature (258)..(258): N = Adenine or Guanine or Cytosine or Thymine
  misc_feature (294)..(294): N = Adenine or Guanine or Cytosine or Thymine
  misc_feature (301)..(301): N = Adenine or Guanine or Cytosine or Thymine
  misc_feature (319)..(319): N = Adenine or Guanine or Cytosine or Thymine
  misc_feature (322)..(322): N = Adenine or Guanine or Cytosine or Thymine
  misc_feature (326)..(326): N = Adenine or Guanine or Cytosine or Thymine
  misc_feature (334)..(334): N = Adenine or Guanine or Cytosine or Thymine
  misc_feature (359)..(359): N = Adenine or Guanine or Cytosine or Thymine
  misc_feature (383)..(383): N = Adenine or Guanine or Cytosine or Thymine
  misc_feature (390)..(390): N = Adenine or Guanine or Cytosine or Thymine
  misc_feature (508)..(508): N = Adenine or Guanine or Cytosine or Thymine
  misc_feature (516)..(517): N = Adenine or Guanine or Cytosine or Thymine
  misc_feature (529)..(529): N = Adenine or Guanine or Cytosine or Thymine
  misc_feature (567)..(567): N = Adenine or Guanine or Cytosine or Thymine
  misc_feature (598)..(598): N = Adenine or Guanine or Cytosine or Thymine
  misc_feature (623)..(626): N = Adenine or Guanine or Cytosine or Thymine
  misc_feature (702)..(702): N = Adenine or Guanine or Cytosine or Thymine
  misc_feature (804)..(804): N = Adenine or Guanine or Cytosine or Thymine
  misc_feature (818)..(818): N = Adenine or Guanine or Cytosine or Thymine
  misc_feature (889)..(889): N = Adenine or Guanine or Cytosine or Thymine
  misc_feature (910)..(912): N = Adenine or Guanine or Cytosine or Thymine
  misc_feature (1002)..(1002): N = Adenine or Guanine or Cytosine or Thymine
atgatatttagacaaaagttagccatgtcataaaaacttngngttggagtgtagtttccaatattacaaaaatatcatngntatctagaatgctatgactttgaaaatatagnttttatgaatgcaaccaaacaccttatgccnnnnannnncactctagnattcctataaaccatgngatngcttagaaactatnaaaaanaatttacttccaaatataccttacgcttagtctttagcttcctntcttcntttttngagtttctacgtgttaagggctggagtctaagtatncatgccnatataattgttgatatanagntccnattcatgnctgttatctaaagaaacaagaaatncatctttgcttcgatcccatgaanccttccntgcccgtcgatgtccaaatttccagctgccccttcaccgcttccctatagctttgcgaagagcctcgctgatcatccagggcacgacggcaccggagccagcaaactgcatagaaatngagcatannagttaaacaacngcgtcagtaaaaaacaaactctccaaccaaagagtctnattaaccagagcagtattgcaatcgccatgnatgtctcttagtttgctggtgatannnncctgagctcgtatcttaagccccatgaagccatctggcgctgaagcctgaaccgagaggacgtccaaatggaggcncttgatggcgtcgaacactcgcgtcatcaggagctcctcccaccggcactgcacctccaggagcacgtccttgtccgagacggtgacggtgacgttgctggngccggcgtccatgntgaggaccggggggcgctccacgtcgtctctgccgagctctgggctcttcctcttggagcccgcgcagacntccttcctcacactctcattnnngccacgggagggccttgttattagccttgtcgtcgtttcggatgggcgcgacgcaggttccctactggactccagctcttgcacccttcnct

SEQ ID NO: 87 - DNA, 1004 bp, Zea mays
  misc_feature (40)..(40): N = Adenine or Guanine or Cytosine or Thymine
  misc_feature (42)..(42): N = Adenine or Guanine or Cytosine or Thymine
  misc_feature (79)..(79): N = Adenine or Guanine or Cytosine or Thymine
  misc_feature (81)..(81): N = Adenine or Guanine or Cytosine or Thymine
  misc_feature (113)..(113): N = Adenine or Guanine or Cytosine or Thymine
  misc_feature (144)..(147): N = Adenine or Guanine or Cytosine or Thymine
  misc_feature (149)..(152): N = Adenine or Guanine or Cytosine or Thymine
  misc_feature (161)..(161): N = Adenine or Guanine or Cytosine or Thymine
  misc_feature (178)..(178): N = Adenine or Guanine or Cytosine or Thymine
  misc_feature (182)..(182): N = Adenine or Guanine or Cytosine or Thymine
  misc_feature (196)..(196): N = Adenine or Guanine or Cytosine or Thymine
  misc_feature (202)..(202): N = Adenine or Guanine or Cytosine or Thymine
  misc_feature (246)..(246): N = Adenine or Guanine or Cytosine or Thymine
  misc_feature (252)..(252): N = Adenine or Guanine or Cytosine or Thymine
  misc_feature (258)..(258): N = Adenine or Guanine or Cytosine or Thymine
  misc_feature (294)..(294): N = Adenine or Guanine or Cytosine or Thymine
  misc_feature (301)..(301): N = Adenine or Guanine or Cytosine or Thymine
  misc_feature (319)..(319): N = Adenine or Guanine or Cytosine or Thymine
  misc_feature (322)..(322): N = Adenine or Guanine or Cytosine or Thymine
  misc_feature (326)..(326): N = Adenine or Guanine or Cytosine or Thymine
  misc_feature (334)..(334): N = Adenine or Guanine or Cytosine or Thymine
  misc_feature (359)..(359): N = Adenine or Guanine or Cytosine or Thymine
  misc_feature (383)..(383): N = Adenine or Guanine or Cytosine or Thymine
  misc_feature (390)..(390): N = Adenine or Guanine or Cytosine or Thymine
  misc_feature (508)..(508): N = Adenine or Guanine or Cytosine or Thymine
  misc_feature (516)..(517): N = Adenine or Guanine or Cytosine or Thymine
  misc_feature (529)..(529): N = Adenine or Guanine or Cytosine or Thymine
  misc_feature (567)..(567): N = Adenine or Guanine or Cytosine or Thymine
  misc_feature (598)..(598): N = Adenine or Guanine or Cytosine or Thymine
  misc_feature (623)..(626): N = Adenine or Guanine or Cytosine or Thymine
  misc_feature (702)..(702): N = Adenine or Guanine or Cytosine or Thymine
  misc_feature (804)..(804): N = Adenine or Guanine or Cytosine or Thymine
  misc_feature (818)..(818): N = Adenine or Guanine or Cytosine or Thymine
  misc_feature (889)..(889): N = Adenine or Guanine or Cytosine or Thymine
  misc_feature (910)..(912): N = Adenine or Guanine or Cytosine or Thymine
  misc_feature (1002)..(1002): N = Adenine or Guanine or Cytosine or Thymine
atgatatttagacaaaagttagccatgtcataaaaacttngngttggagtgtagtttccaatattacaaaaatatcatngntatctagaatgctatgactttgaaaatatagnttttatgaatgcaaccaaacaccttatgccnnnnannnncactctagnattcctataaaccatgngatngcttagaaactatnaaaaanaatttacttccaaatataccttacgcttagtctttagcttcctntcttcntttttngagtttctacgtgttaagggctggagtctaagtatncatgccnatataattgttgatatanagntccnattcatgnctgttatctaaagaaacaagaaatncatctttgcttcgatcccatgaanccttccntgcccgtcgatgtccaaatttccagctgccccttcaccgcttccctatagctttgcgaagagcctcgctgatcatccagggcacgacggcaccggagccagcaaactgcataggaatngagcatannagttaaacaacngcgtcagtaaaaaacaaactctccaaccaaagagtctnattaaccagagcagtattgcaatcgccatgnatgtctcttagtttgctggtgatannnncctgagctcgtatcttaagccccatgaagccatctggcgctgaagcctgaaccgagaggacgtccaaatggaggcncttgatggcgtcgaacactcgcgtcatcaggagctcctcccaccggcactgcacctccaggagcacgtccttgtccgagacggtgacggtgacgttgctggngccggcgtccatgntgaggaccggggggcgctccacgtcgtctctgccgagctctgggctcttcctcttggagcccgcgcagacntccttcctcacactctcattnnngccacgggagggccttgttattagccttgtcgtcgtttcggatgggcgcgacgcaggttccctactggactccagctcttgcacccttcnct

SEQ ID NO: 88 - DNA, 19 bp, Artificial Sequence
  SM8040 F primer
ccctgttccgaggccattt

SEQ ID NO: 89 - DNA, 20 bp, Artificial Sequence
  SM8040 R primer
cgtgagctttcgtctgcgat

SEQ ID NO: 90 - DNA, 17 bp, Artificial Sequence
  SM8040 FAM
tcgatcgggtatgctct

SEQ ID NO: 91 - DNA, 14 bp, Artificial Sequence
  SM8040 TET
cgggtgtgctctcc

SEQ ID NO: 92 - DNA, 1022 bp, Zea mays
  misc_feature (54)..(54): N = Adenine or Guanine or Cytosine or Thymine
  misc_feature (99)..(100): N = Adenine or Guanine or Cytosine or Thymine
  misc_feature (102)..(103): N = Adenine or Guanine or Cytosine or Thymine
  misc_feature (141)..(141): N = Adenine or Guanine or Cytosine or Thymine
  misc_feature (156)..(156): N = Adenine or Guanine or Cytosine or Thymine
  misc_feature (160)..(163): N = Adenine or Guanine or Cytosine or Thymine
  misc_feature (174)..(177): N = Adenine or Guanine or Cytosine or Thymine
  misc_feature (182)..(182): N = Adenine or Guanine or Cytosine or Thymine
  misc_feature (188)..(189): N = Adenine or Guanine or Cytosine or Thymine
  misc_feature (203)..(203): N = Adenine or Guanine or Cytosine or Thymine
  misc_feature (229)..(232): N = Adenine or Guanine or Cytosine or Thymine
  misc_feature (246)..(246): N = Adenine or Guanine or Cytosine or Thymine
  misc_feature (253)..(254): N = Adenine or Guanine or Cytosine or Thymine
  misc_feature (261)..(272): N = Adenine or Guanine or Cytosine or Thymine
  misc_feature (275)..(278): N = Adenine or Guanine or Cytosine or Thymine
  misc_feature (422)..(422): N = Adenine or Guanine or Cytosine or Thymine
  misc_feature (531)..(533): N = Adenine or Guanine or Cytosine or Thymine
gcgacgaggttgcggtagacgacgaggacgaagtggttcatgccgcgcttgagngaggcgacggagacgatgtacatccccgagaaccccacctgcagnngnnccatcgccaggtacggcttcacgtcgttcatcagcttncccacgcccatggcngctnnnngtacgtacgtnnnncacgncccccnntctgatcgattacnagcacgacgtgctgagggagggaggnnnncaaggcaggcaggngtggcgnnagctagnnnnnnnnnnnnctnnnncacttgtgctgctcctctcctcgatctactccacggcgaggaagacagcccgctgcgctgccggttttatagcaatgcgcgagcgcgcggcacacgtcaggtcaatgtcgtcccatgcatgcatgccccccacccccacccccncgcgcgcgcaacgtcacgaacgcagttgcaggttgcagcataccccagcactcttgccctgttccgaggccattttccgcgccacatcgatcgggtgtgctctccactnnnagctcaaaaaagaaaaaaaaaagataattaattacgcgctctcttctccatcgcagacgaaagctcacgtctctatagttgcatatatgtgatgttaacacacgtgaacagacgaggtcttcacggtaaataccacatcgtcacacaaataccatatatatacgtacgtgctacccggagaggcgcgtgatgtaactataacacctctaggcatatataccggcgtgtacgtacggtatcagaaactatagcttgttcgattttgacagcctcataaccaaagaaagctcgtctagcgggagaggataatgggatctaaattttacgctataaaatttaaggattcgatcgaattaagattagactctgtttatatttatttttgaactaaaagtaattaagagctcaaacaatttgggaaaaaaatatttggaccgtgatccaataccacccctaagtcgggagggagactccgctaggagaatcgcg

SEQ ID NO: 93 - DNA, 1022 bp, Zea mays
  misc_feature (54)..(54): N = Adenine or Guanine or Cytosine or Thymine
  misc_feature (99)..(100): N = Adenine or Guanine or Cytosine or Thymine
  misc_feature (102)..(103): N = Adenine or Guanine or Cytosine or Thymine
  misc_feature (141)..(141): N = Adenine or Guanine or Cytosine or Thymine
  misc_feature (156)..(156): N = Adenine or Guanine or Cytosine or Thymine
  misc_feature (160)..(163): N = Adenine or Guanine or Cytosine or Thymine
  misc_feature (174)..(177): N = Adenine or Guanine or Cytosine or Thymine
  misc_feature (182)..(182): N = Adenine or Guanine or Cytosine or Thymine
  misc_feature (188)..(189): N = Adenine or Guanine or Cytosine or Thymine
  misc_feature (203)..(203): N = Adenine or Guanine or Cytosine or Thymine
  misc_feature (229)..(232): N = Adenine or Guanine or Cytosine or Thymine
  misc_feature (246)..(246): N = Adenine or Guanine or Cytosine or Thymine
  misc_feature (253)..(254): N = Adenine or Guanine or Cytosine or Thymine
  misc_feature (261)..(272): N = Adenine or Guanine or Cytosine or Thymine
  misc_feature (275)..(278): N = Adenine or Guanine or Cytosine or Thymine
  misc_feature (422)..(422): N = Adenine or Guanine or Cytosine or Thymine
  misc_feature (531)..(533): N = Adenine or Guanine or Cytosine or Thymine
gcgacgaggttgcggtagacgacgaggacgaagtggttcatgccgcgcttgagngaggcgacggagacgatgtacatccccgagaaccccacctgcagnngnnccatcgccaggtacggcttcacgtcgttcatcagcttncccacgcccatggcngctnnnngtacgtacgtnnnncacgncccccnntctgatcgattacnagcacgacgtgctgagggagggaggnnnncaaggcaggcaggngtggcgnnagctagnnnnnnnnnnnnctnnnncacttgtgctgctcctctcctcgatctactccacggcgaggaagacagcccgctgcgctgccggttttatagcaatgcgcgagcgcgcggcacacgtcaggtcaatgtcgtcccatgcatgcatgccccccacccccacccccncgcgcgcgcaacgtcacgaacgcagttgcaggttgcagcataccccagcactcttgccctgttccgaggccattttccgcgccacatcgatcgggtatgctctccactnnnagctcaaaaaagaaaaaaaaaagataattaattacgcgctctcttctccatcgcagacgaaagctcacgtctctatagttgcatatatgtgatgttaacacacgtgaacagacgaggtcttcacggtaaataccacatcgtcacacaaataccatatatatacgtacgtgctacccggagaggcgcgtgatgtaactataacacctctaggcatatataccggcgtgtacgtacggtatcagaaactatagcttgttcgattttgacagcctcataaccaaagaaagctcgtctagcgggagaggataatgggatctaaattttacgctataaaatttaaggattcgatcgaattaagattagactctgtttatatttatttttgaactaaaagtaattaagagctcaaacaatttgggaaaaaaatatttggaccgtgatccaataccacccctaagtcgggagggagactccgctaggagaatcgcg

SEQ ID NO: 94 - DNA, 18 bp, Artificial Sequence
  SM8091 F primer
ctgcacgccgccagatta

SEQ ID NO: 95 - DNA, 22 bp, Artificial Sequence
  SM8091 R primer
cagctgacggcaacaattagta

SEQ ID NO: 96 - DNA, 18 bp, Artificial Sequence
  SM8091 FAM
cgaccgtaaccatgtaac

SEQ ID NO: 97 - DNA, 17 bp, Artificial Sequence
  SM8091 TET
accgtaaccctgtaaca

SEQ ID NO: 98 - DNA, 1002 bp, Zea mays
  misc_feature (6)..(6): N = Adenine or Guanine or Cytosine or Thymine
  misc_feature (27)..(27): N = Adenine or Guanine or Cytosine or Thymine
  misc_feature (31)..(31): N = Adenine or Guanine or Cytosine or Thymine
  misc_feature (36)..(36): N = Adenine or Guanine or Cytosine or Thymine
  misc_feature (66)..(66): N = Adenine or Guanine or Cytosine or Thymine
  misc_feature (78)..(78): N = Adenine or Guanine or Cytosine or Thymine
  misc_feature (84)..(84): N = Adenine or Guanine or Cytosine or Thymine
  misc_feature (101)..(101): N = Adenine or Guanine or Cytosine or Thymine
  misc_feature (119)..(119): N = Adenine or Guanine or Cytosine or Thymine
  misc_feature (131)..(131): N = Adenine or Guanine or Cytosine or Thymine
  misc_feature (137)..(137): N = Adenine or Guanine or Cytosine or Thymine
  misc_feature (140)..(153): N = Adenine or Guanine or Cytosine or Thymine
  misc_feature (157)..(157): N = Adenine or Guanine or Cytosine or Thymine
  misc_feature (172)..(172): N = Adenine or Guanine or Cytosine or Thymine
  misc_feature (174)..(174): N = Adenine or Guanine or Cytosine or Thymine
  misc_feature (215)..(215): N = Adenine or Guanine or Cytosine or Thymine
  misc_feature (218)..(218): N = Adenine or Guanine or Cytosine or Thymine
  misc_feature (250)..(250): N = Adenine or Guanine or Cytosine or Thymine
  misc_feature (266)..(266): N = Adenine or Guanine or Cytosine or Thymine
  misc_feature (279)..(279): N = Adenine or Guanine or Cytosine or Thymine
  misc_feature (285)..(285): N = Adenine or Guanine or Cytosine or Thymine
  misc_feature (287)..(287): N = Adenine or Guanine or Cytosine or Thymine
  misc_feature (292)..(292): N = Adenine or Guanine or Cytosine or Thymine
  misc_feature (420)..(420): N = Adenine or Guanine or Cytosine or Thymine
  misc_feature (453)..(453): N = Adenine or Guanine or Cytosine or Thymine
  misc_feature (572)..(572): N = Adenine or Guanine or Cytosine or Thymine
  misc_feature (619)..(619): N = Adenine or Guanine or Cytosine or Thymine
  misc_feature (820)..(820): N = Adenine or Guanine or Cytosine or Thymine
  misc_feature (842)..(842): N = Adenine or Guanine or Cytosine or Thymine
  misc_feature (870)..(870): N = Adenine or Guanine or Cytosine or Thymine
  misc_feature (880)..(884): N = Adenine or Guanine or Cytosine or Thymine
caaaangtgttagtacattttaaaggnaagnccaangatggataatcttctgaatacatgagttgncatcgtccttcncctggngaccaatggtactctcnacctcccagaagtagaantgtttgttcagnattttnttnnnnnnnnnnnnnnatantcggtcaatactatncntagaatgggccggtggctttaggcctatatcggtactatgnatngaggcccaggttcagcctagatgcatatcagntagccctccctcgtcngtcgatattattnttctcngntgttnttttgcaggtcggggggggggggggagtagttgaatggtgctgatttttttggtagaaagctaaaaaaactgacgaccctgcatatcaactaaagtagcacgtagaaatcttgggaagccggtacagntcacaatttggttccacacgcgaaaagcatcangacacttgcatgtgcctgcacgccgccagattattacgaccgtaaccctgtaacaaaatttcaaatactaattgttgccgtcagctgacgtaagctatagaggatttgggaatccttcnttaacgcaaatatgcctaattttttttttgtagttggtctttttttngaaacaaattcatgtttagacctttgacgatttaatgtcgtttttaggccttttgctgggcgctataaatcatgacgacaatataatatgtctcggcgtcatgacttataatgtcgaggtaatagatacacactggactactcatgctacgatggcgtctatgcttagcttggcgtcatagatcacggcgccgagctcacngctgacgattgcaacgcttccngcggcgagaattgatgccgcatccaacntgcatgtctnnnnngcggtctcatccttcatgcatgagttcttttctttgtcaaaaataatggacgttgatggcagcaactctagtcccaccgcatccatagcacatcaccgtgatttatttccgtacgaag

SEQ ID NO: 99 - DNA, 1002 bp, Zea mays
  misc_feature (6)..(6): N = Adenine or Guanine or Cytosine or Thymine
  misc_feature (27)..(27): N = Adenine or Guanine or Cytosine or Thymine
  misc_feature (31)..(31): N = Adenine or Guanine or Cytosine or Thymine
  misc_feature (36)..(36): N = Adenine or Guanine or Cytosine or Thymine
  misc_feature (66)..(66): N = Adenine or Guanine or Cytosine or Thymine
  misc_feature (78)..(78): N = Adenine or Guanine or Cytosine or Thymine
  misc_feature (84)..(84): N = Adenine or Guanine or Cytosine or Thymine
  misc_feature (101)..(101): N = Adenine or Guanine or Cytosine or Thymine
  misc_feature (119)..(119): N = Adenine or Guanine or Cytosine or Thymine
  misc_feature (131)..(131): N = Adenine or Guanine or Cytosine or Thymine
  misc_feature (137)..(137): N = Adenine or Guanine or Cytosine or Thymine
  misc_feature (140)..(153): N = Adenine or Guanine or Cytosine or Thymine
  misc_feature (157)..(157): N = Adenine or Guanine or Cytosine or Thymine
  misc_feature (172)..(172): N = Adenine or Guanine or Cytosine or Thymine
  misc_feature (174)..(174): N = Adenine or Guanine or Cytosine or Thymine
  misc_feature (215)..(215): N = Adenine or Guanine or Cytosine or Thymine
  misc_feature (218)..(218): N = Adenine or Guanine or Cytosine or Thymine
  misc_feature (250)..(250): N = Adenine or Guanine or Cytosine or Thymine
  misc_feature (266)..(266): N = Adenine or Guanine or Cytosine or Thymine
  misc_feature (279)..(279): N = Adenine or Guanine or Cytosine or Thymine
  misc_feature (285)..(285): N = Adenine or Guanine or Cytosine or Thymine
  misc_feature (287)..(287): N = Adenine or Guanine or Cytosine or Thymine
  misc_feature (292)..(292): N = Adenine or Guanine or Cytosine or Thymine
  misc_feature (420)..(420): N = Adenine or Guanine or Cytosine or Thymine
  misc_feature (453)..(453): N = Adenine or Guanine or Cytosine or Thymine
  misc_feature (572)..(572): N = Adenine or Guanine or Cytosine or Thymine
  misc_feature (619)..(619): N = Adenine or Guanine or Cytosine or Thymine
  misc_feature (820)..(820): N = Adenine or Guanine or Cytosine or Thymine
  misc_feature (842)..(842): N = Adenine or Guanine or Cytosine or Thymine
  misc_feature (870)..(870): N = Adenine or Guanine or Cytosine or Thymine
  misc_feature (880)..(884): N = Adenine or Guanine or Cytosine or Thymine
caaaangtgttagtacattttaaaggnaagnccaangatggataatcttctgaatacatgagttgncatcgtccttcncctggngaccaatggtactctcnacctcccagaagtagaantgtttgttcagnattttnttnnnnnnnnnnnnnnatantcggtcaatactatncntagaatgggccggtggctttaggcctatatcggtactatgnatngaggcccaggttcagcctagatgcatatcagntagccctccctcgtcngtcgatattattnttctcngntgttnttttgcaggtcggggggggggggggagtagttgaatggtgctgatttttttggtagaaagctaaaaaaactgacgaccctgcatatcaactaaagtagcacgtagaaatcttgggaagccggtacagntcacaatttggttccacacgcgaaaagcatcangacacttgcatgtgcctgcacgccgccagattattacgaccgtaaccatgtaacaaaatttcaaatactaattgttgccgtcagctgacgtaagctatagaggatttgggaatccttcnttaacgcaaatatgcctaattttttttttgtagttggtctttttttngaaacaaattcatgtttagacctttgacgatttaatgtcgtttttaggccttttgctgggcgctataaatcatgacgacaatataatatgtctcggcgtcatgacttataatgtcgaggtaatagatacacactggactactcatgctacgatggcgtctatgcttagcttggcgtcatagatcacggcgccgagctcacngctgacgattgcaacgcttccngcggcgagaattgatgccgcatccaacntgcatgtctnnnnngcggtctcatccttcatgcatgagttcttttctttgtcaaaaataatggacgttgatggcagcaactctagtcccaccgcatccatagcacatcaccgtgatttatttccgtacgaag

SEQ ID NO: 100 - DNA, 25 bp, Artificial Sequence
  SM2918 F primer
atggtgccaattcgtaatttaagtt

SEQ ID NO: 101 - DNA, 20 bp, Artificial Sequence
  SM2918 R primer
acccctctggttgcctctct

SEQ ID NO: 102 - DNA, 16 bp, Artificial Sequence
  SM2918 FAM
aataccctccagtttc

SEQ ID NO: 103 - DNA, 20 bp, Artificial Sequence
  SM2918 TET
tatgaagaagaataccatcc

SEQ ID NO: 104 - DNA, 424 bp, Zea mays
  misc_feature (268)..(268): N = Adenine or Guanine or Cytosine or Thymine
atgatgatgatgactagatggagttccactgatatgaagagaagaaatagaatattggctaatatggtgccaattcgtaatttaagtttacctgattattatgaatatgaagaagaataccctccagtttcaagagaggcaaccagaggggtctgtatactcctacgaatagacagatatttatcttcaattggaaggagcattcaagaccgtgaggttctacgcgatttccgccaacggttactctttccccaacgcgaggctgggnacagcttttccgaaatatatgatgatatacgagcgcatggggtagaagcaagtcgattgggtcagcctctaagagatctgtacgatgagatggaaaggaacggcgagatagtaaataacggctcaatcattatccctggaggcggcggaccagtaa

SEQ ID NO: 105 - DNA, 424 bp, Zea mays
  misc_feature (268)..(268): N = Adenine or Guanine or Cytosine or Thymine
atgatgatgatgactagatggagttccactgatatgaagagaagaaatagaatattggctaatatggtgccaattcgtaatttaagtttacctgattattatgaatatgaagaagaataccatccagtttcaagagaggcaaccagaggggtctgtatactcctacgaatagacagatatttatcttcaattggaaggagcattcaagaccgtgaggttctacgcgatttccgccaacggttactctttccccaacgcgaggctgggnacagcttttccgaaatatatgatgatatacgagcgcatggggtagaagcaagtcgattgggtcagcctctaagagatctgtacgatgagatggaaaggaacggcgagatagtaaataacggctcaatcattatccctggaggcggcggaccagtaa

SEQ ID NO: 106 - DNA, 23 bp, Artificial Sequence
  SM4813 F primer
ggctaatatggtgccaattcgta

SEQ ID NO: 107 - DNA, 23 bp, Artificial Sequence
  SM4813 R primer
ggagtatacagacccctctggtt

SEQ ID NO: 108 - DNA, 20 bp, Artificial Sequence
  SM4813 FAM
ttgaaactggagggtattct

SEQ ID NO: 109 - DNA, 20 bp, Artificial Sequence
  SM4813 TET
cttgaaactggatggtattc

SEQ ID NO: 110 - DNA, 424 bp, Zea mays; identical to SEQ ID NO: 104
  misc_feature (268)..(268): N = Adenine or Guanine or Cytosine or Thymine

SEQ ID NO: 111 - DNA, 424 bp, Zea mays; identical to SEQ ID NO: 105
  misc_feature (268)..(268): N = Adenine or Guanine or Cytosine or Thymine

SEQ ID NO: 112 - DNA, 21 bp, Artificial Sequence
  SM2914 F primer
gtattcgcacctactctgccg

SEQ ID NO: 113 - DNA, 23 bp, Artificial Sequence
  SM2914 R primer
aaagcaaaaataccattgcaacc

SEQ ID NO: 114 - DNA, 19 bp, Artificial Sequence
  SM2914 FAM
cgtaaattttgtttgatgc

SEQ ID NO: 115 - DNA, 21 bp, Artificial Sequence
  SM2914 TET
atcgtaaattttttttgatgc

SEQ ID NO: 116 - DNA, 616 bp, Zea mays
  misc_feature (382)..(382): N = Adenine or Guanine or Cytosine or Thymine
  misc_feature (402)..(402): N = Adenine or Guanine or Cytosine or Thymine
  misc_feature (526)..(526): N = Adenine or Guanine or Cytosine or Thymine
gatgcctttttaaggaaccctccccccgagtcggatttgtctcaggaagtggggccggttccaccgccaccggtggcgtgttgtcatggggatgatcacccccttctggcgaagggccagtggttgggctcaaccgttcgccctccgtagggctgagtggcggatctataaaatctggggcggaccgaacctccatttcgaattcaaaatctaaaggagcctttcctttgcgcccctcttgggtggatccctcccccgcttccattagaagcggtttttgcccccccacctttgaaggcagtttatcggaagaaccccaactttctccttgggtcatccccagagtagcactctgctcccataggagctggaatagaaaacngagccacattacgagtgcgncaatgagagccttccggagcgccagaaagagttccgcgagatcaaaaaagcaaaaataccattgcaaccaacacgaggcaggaagcagaagcatcaaacaaaatttacgattcatgacaatccnatttttgaagcgaaaaaacttcttcttccacttaggagaggcggcagagtaggtgcgaatacccagaagggtactcgctcctcgtgctcc

SEQ ID NO: 117 - DNA, 616 bp, Zea mays
  misc_feature (382)..(382): N = Adenine or Guanine or Cytosine or Thymine
  misc_feature (402)..(402): N = Adenine or Guanine or Cytosine or Thymine
  misc_feature (526)..(526): N = Adenine or Guanine or Cytosine or Thymine
gatgcctttttaaggaaccctccccccgagtcggatttgtctcaggaagtggggccggttccaccgccaccggtggcgtgttgtcatggggatgatcacccccttctggcgaagggccagtggttgggctcaaccgttcgccctccgtagggctgagtggcggatctataaaatctggggcggaccgaacctccatttcgaattcaaaatctaaaggagcctttcctttgcgcccctcttgggtggatccctcccccgcttccattagaagcggtttttgcccccccacctttgaaggcagtttatcggaagaaccccaactttctccttgggtcatccccagagtagcactctgctcccataggagctggaatagaaaacngagccacattacgagtgcgncaatgagagccttccggagcgccagaaagagttccgcgagatcaaaaaagcaaaaataccattgcaaccaacacgaggcaggaagcagaagcatcaaaaaaaatttacgattcatgacaatccnatttttgaagcgaaaaaacttcttcttccacttaggagaggcggcagagtaggtgcgaatacccagaagggtactcgctcctcgtgctcc

SEQ ID NO: 118 - DNA, 25 bp, Artificial Sequence
  SM4812 F primer
ggctttatagtcctcagaaaggtga

SEQ ID NO: 119 - DNA, 21 bp, Artificial Sequence
  SM4812 R primer
tcgtagccacgtgctctaatc

SEQ ID NO: 120 - DNA, 18 bp, Artificial Sequence
  SM4812 FAM
cgggaagagatcctgtgg

SEQ ID NO: 121 - DNA, 14 bp, Artificial Sequence
  SM4812 TET
cccgggacctgtgg

SEQ ID NO: 122 - DNA, 1005 bp, Zea mays
tctcatattaagtcggaagagtttaggtaagactttcaaagaaactctcgacgggagaatcgagtctattcaggaatcattgcagcaattcttcaatcctaacgaagtcattctggaggaatccaatgaacaacaacgattacttaatctacggatcagcttgcgaatttgcagcaccgtaaaagtagtagaatcattaccagcggcacgctgtgcgcctaagtgcgaaaagacagtgcaagctttgttatgccgaaacctaaatgtcaagtcagcaacacttctaaatgccacttcttcccgtcgcatccgtcttcaggacgatatagtcacaggttttcacttttcagtgagtgaaagattagtatccgggtctacaactttggtagaagcttctaccgtagaacaaattcgagaggccttcttattagaacccagagacctaattcgagaaggctttatagtcctcagaaaggtgagggtggggggtatccccgggaagagatcctgtggagacggggtgggcctgtagctcagctcagaggattagagcacgtggctacgaaccacgatgttgggggttcgaatccacttctgagaaggaagtattggctatgcaccccttcccttgactaactaagtctgcatataaggagtgcagaaattctatactgttcgtatcgccgtgcaaattatacgcaatttgtttcattgtaaacccttggcccttccaattcacgcccggaggagtattacaatagacgttgaaacaaccctggagcttatctgtaatttgctccttgagcgtttctaacgtcaataaagtcctccaacttatgatgccagttttccgaagccgcggcttttacccgctttataagcgatgagtagggcgatgcataaaaagtcatatttcttggtgtagggatggatctcataggaaaagagataccgaggcccaccaaccatatacttgatttatggtttggtggggaaagaagagtg

SEQ ID NO: 123 - DNA, 999 bp, Zea mays
tctcatattaagtcggaagagtttaggtaagactttcaaagaaactctcgacgggagaatcgagtctattcaggaatcattgcagcaattcttcaatcctaacgaagtcattctggaggaatccaatgaacaacaacgattacttaatctacggatcagcttgcgaatttgcagcaccgtaaaagtagtagaatcattaccagcggcacgctgtgcgcctaagtgcgaaaagacagtgcaagctttgttatgccgaaacctaaatgtcaagtcagcaacacttctaaatgccacttcttcccgtcgcatccgtcttcaggacgatatagtcacaggttttcacttttcagtgagtgaaagattagtatccgggtctacaactttggtagaagcttctaccgtagaacaaattcgagaggccttcttattagaacccagagacctaattcgagaaggctttatagtcctcagaaaggtgagggtggggggtatccccgggacctgtggagacggggtgggcctgtagctcagctcagaggattagagcacgtggctacgaaccacgatgttgggggttcgaatccacttctgagaaggaagtattggctatgcaccccttcccttgactaactaagtctgcatataaggagtgcagaaattctatactgttcgtatcgccgtgcaaattatacgcaatttgtttcattgtaaacccttggcccttccaattcacgcccggaggagtattacaatagacgttgaaacaaccctggagcttatctgtaatttgctccttgagcgtttctaacgtcaataaagtcctccaacttatgatgccagttttccgaagccgcggcttttacccgctttataagcgatgagtagggcgatgcataaaaagtcatatttcttggtgtagggatggatctcataggaaaagagataccgaggcccaccaaccatatacttgatttatggtttggtggggaaagaagagtg

SEQ ID NO: 124 - DNA, 1371 bp, Zea mays
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggaggtacgaaccggtgactggcgaaggaagcaatgccgatgccctcggtgggctcgctaggcagctctccgaggagaggagaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 125 - DNA, 1375 bp, Zea mays
agttcatcactaatcacacttattgttccctcgacgagtatctagctagctcattaatcgatcaatcggggtgtgcggtcgaaggcggcaatggcgagctactcgtcgcggcgtccatgcaatacctgtagcacgaaggcgatggccgggagcgtggtcggcgagcccgtcgtgctggggcagagggtgacggtgctgacggtggacggcggcggcgtccggggtctcatcccgggaaccatcctcgccttcctcgaggccaggctgcaggagctggacggaccggaggcgaggctggcggactacttcgactacatcgccggaaccagcaccggcggtctcatcaccgccatgctcaccgcgcccggcaaggacaagcggcctctctacgctgccaaggacatcaaccacttttacatggagaactgcccgcgcatcttccctcagaagagcaggcttgcggccgccatgtccgcgctgaggaagccaaagtacaacggcaagtgcatgcgcagcctgattaggagcatcctcggcgagacgagggccaagagcacgcctctgaagaacgctctgctctcggacgtgtgcattggcacgtccgccgcgccgacctacctcccggcgcactacttccagactgaagacgccaacggcaaggagcgcgaatacaacctcatcgacggcggtgtggcggccaacaacccgacgatggttgcgatgacgcagatcaccaaaaagatgcttgccagcaaggacaaggccgaggagctgtacccagtgaagccgtcgaactgccgcaggttcctggtgctgtccatcgggacggggtcgacgtccgagcagggcctctacacggcgcggcagtgctcccggtggggtatctgccggtggctccgcaacaacggcatggcccccatcatcgacatcttcatggcggccagctcggacctggtggacatccacgtcgccgcgatgttccagtcgctccacagcgacggcgactacctgcgcatccaggacaactcgctccgtggcgccgcggccaccgtggacgcggcgacgccggagaacatgcggacgctcgtcgggatcggggagcggatgctggcacagagggtgtccagggtcaacgtggagacagggagcgaggtacgaaccggtgactggcgaaggaagcaatgccgatgccctcggtgggctcgctaggcagctctccgaggagaggagaacaaggctcgcgcgccgcgtgtctgccatcaacccaagaggctctagatgtgcgtcgtacgatatctaagacaagtggctttactgtcagtcacatgcttgtaaataagtagactttattttaataaaacataaaaatatatat

SEQ ID NO: 126 - DNA, 618 bp, Zea mays
atggatcgcagcaacgccggtgcggtgtccgtcgaggtgcgcggcggcggcggcggctcgccgccgggcgcgggaaggaagcgccgcgcggtggcgaggggcgtgcagaagacgctctccaagacgtccatgctggccaacttcctccccacgggcacgctgctaaccttcgagatgctactcccggccgccgcaggcgacggcacctgctcggcggtcagcgccgcgatgctcagggccctgctcgcgctctgcgccgcctcctgcttcctcttccacttcaccgacagcttccgcgccccggacgggaaggtgtactacggcttcgtcacgccgcggggcctgtcgctgttcaggaccgggctcggcgtcgaggtgcccagggaggaaaggtaccggctcgccttcgtcgacgtcgtgcacgctgtcatgtccgtgctggtctttgcggccgtcacgctcgccgactaccgggtctccgggtgcctcgtcgccggccaccgcaaggagatggacgaggtgatggagagcttcccgctcatggtgggcgccgtgtgcagcggcctcttcctcttgttccccaacacccgctacggcatcggttgtttggctccgtaa

SEQ ID NO: 127 - DNA, 21 bp, Artificial Sequence
  Waxy1 probe
ggtttcaggtttggggaaaga

SEQ ID NO: 128 - DNA, 23 bp, Artificial Sequence
  Waxy1 gRNA target sequence
gggaaagaccgaggagaagatct

SEQ ID NO: 129 - DNA, 22 bp, Artificial Sequence
  SM3158 F primer
caaaccaggaaccaagctcact

SEQ ID NO: 130 - DNA, 21 bp, Artificial Sequence
  SM3158 R primer
ggaccacgacatcttgaagga

SEQ ID NO: 131 - DNA, 17 bp, Artificial Sequence
  SM3158 FAM
cggcctacactaggagt

SEQ ID NO: 132 - DNA, 16 bp, Artificial Sequence
  SM3158 TET
cctacactgggagtcc

SEQ ID NO: 133 - DNA, 1001 bp, Zea mays
cgcttctctgccttggaggatgaattagcaagggaagtcgccaatacctttaatttctcaacctgcatcattttagtccattatgttagtatatagctatattttcaccaatgtccatgtatataagcatagttcatctagaatgattactatcagcgtccggggaagaccataccctcggatactgacaatatgcctatcaaagatcacatcaacagagaaaagctcaatcagggctcggcccaaccatgtgtccctaaggccctgtgcccaaaggggtcttgctcggctccagataagtcagacgccctccacatcggagaccaggtcccgaggccctctagccctcgaagctcttccgcctcaagcaccctccataccgggggactagatcacggggccctcaggacctctgaggcccagggccttccagcctctagagccctatttctctcgaggctctccaaaccaggaaccaagctcactgctcggcctacactaggagtccctgtaggctaagatctacaactagatctctcgggcatccttcaagatgtcgtggtcccaacagtcgaagcccctagggtaggacaaccccgaacgaagatgcctcccaaggcagtaccaccctagtgccatgtgaacccctactcaagacgaccataccgggtagttaggattaaggctagctccagcaacggaccctaaagggttctgtaccctaaatatagaggatcaaatggttctctacgctctccagcaacgtcctctaaacggtcctctaaatttagaggacgctgctggattctctatatatagagtttctctaaacggtactctatccatttgaatactttaaataaccggtttagcaaaactaaaatatgtacaatacatttgagagtataacaaatacgtatgtacaaaaaataaaaataaaaaatgtctctaatatagatatttgagtatagaggacgtgatttagaggacgttgctggaga

SEQ ID NO: 134 - DNA, 1001 bp, Zea mays
cgcttctctgccttggaggatgaattagcaagggaagtcgccaatacctttaatttctcaacctgcatcattttagtccattatgttagtatatagctatattttcaccaatgtccatgtatataagcatagttcatctagaatgattactatcagcgtccggggaagaccataccctcggatactgacaatatgcctatcaaagatcacatcaacagagaaaagctcaatcagggctcggcccaaccatgtgtccctaaggccctgtgcccaaaggggtcttgctcggctccagataagtcagacgccctccacatcggagaccaggtcccgaggccctctagccctcgaagctcttccgcctcaagcaccctccataccgggggactagatcacggggccctcaggacctctgaggcccagggccttccagcctctagagccctatttctctcgaggctctccaaaccaggaaccaagctcactgctcggcctacactgggagtccctgtaggctaagatctacaactagatctctcgggcatccttcaagatgtcgtggtcccaacagtcgaagcccctagggtaggacaaccccgaacgaagatgcctcccaaggcagtaccaccctagtgccatgtgaacccctactcaagacgaccataccgggtagttaggattaaggctagctccagcaacggaccctaaagggttctgtaccctaaatatagaggatcaaatggttctctacgctctccagcaacgtcctctaaacggtcctctaaatttagaggacgctgctggattctctatatatagagtttctctaaacggtactctatccatttgaatactttaaataaccggtttagcaaaactaaaatatgtacaatacatttgagagtataacaaatacgtatgtacaaaaaataaaaataaaaaatgtctctaatatagatatttgagtatagaggacgtgatttagaggacgttgctggaga

SEQ ID NO: 135 - DNA, 20 bp, Artificial Sequence
  SM4787 F primer
cgctggcatgaactccactc

SEQ ID NO: 136 - DNA, 20 bp, Artificial Sequence
  SM4787 R primer
tggtgccttgacctggtatg

SEQ ID NO: 137 - DNA, 15 bp, Artificial Sequence
  SM4787 FAM
tgtgcgacgatgtcg

SEQ ID NO: 138 - DNA, 19 bp, Artificial Sequence
  SM4787 TET
tgatttgtgcgatgatgtc

SEQ ID NO: 139 - DNA, 1005 bp, Zea mays
  misc_feature (825)..(825): N = Adenine or Guanine or Cytosine or Thymine
  misc_feature (868)..(868): N = Adenine or Guanine or Cytosine or Thymine
  misc_feature (894)..(894): N = Adenine or Guanine or Cytosine or Thymine
  misc_feature (896)..(896): N = Adenine or Guanine or Cytosine or Thymine
  misc_feature (912)..(912): N = Adenine or Guanine or Cytosine or Thymine
  misc_feature (935)..(937): N = Adenine or Guanine or Cytosine or Thymine
  misc_feature (956)..(956): N = Adenine or Guanine or Cytosine or Thymine
  misc_feature (960)..(960): N = Adenine or Guanine or Cytosine or Thymine
  misc_feature (983)..(983): N = Adenine or Guanine or Cytosine or Thymine
gaaggataaatagggccgtagggccatcgttttgcattgccgtcgcgtggccgtgggttttcatcgacgaacgacgacgactctgccgcgcgcgtttaggcgcgtcgccgccggccacccggccggccacttcgacgcggctgtggtggtttaaaattctccggctcgccgcccacggcctcgctgcacgtagttgcaggtgagtgaggttcttcgattccccccaaacccggcccatctccgacgatccattatttgttcgtccatatgaaaatgcatctctttttgtacccagttgttacctacatgcagcttttactgcttcattcagttatggtcgtgtacatcttcagaaaaccagacatgttcaggcaagaccagtgggtatgcaaaaacaacagcaaagtggctcggcatgtcgacagccagctgctacttctacttctacatataaaccacgcgctggcatgaactccactccagcatggatagacgacatcatcgcacaaatcacataccaggtcaaggcaccacgtttcacaaacctagtgaacacccaagaactattatgcaataccatcattttgctcgatagattttcctagcccagttgtttcatctgagcaagtgtctgtaggaactaaaacacagtagtatgctgtatatactgtcaagcatgaaacgcaaccagatcagctgcaggatttgcagcaccaagctctggccattaggtaggctgcatatcacaacacacaaagggaacatcaaactacatacactagctccgtcccctccagggacactgcaggttggcaggcccaaagntagcacagggtctcgcaacccacaaggtcataatcagtacaancatccatcggcaaggggaagcaagancnaatcccaggtaggtgnacgctggacagatccgtgctcannncgccgttccttgctccggncttntctggccgcttgtactagctccnctgttttgtttcgccaacatct

SEQ ID NO: 140 - DNA, 1005 bp, Zea mays
  misc_feature (825)..(825): N = Adenine or Guanine or Cytosine or Thymine
  misc_feature (868)..(868): N = Adenine or Guanine or Cytosine or Thymine
  misc_feature (894)..(894): N = Adenine or Guanine or Cytosine or Thymine
  misc_feature (896)..(896): N = Adenine or Guanine or Cytosine or Thymine
  misc_feature (912)..(912): N = Adenine or Guanine or Cytosine or Thymine
  misc_feature (935)..(937): N = Adenine or Guanine or Cytosine or Thymine
  misc_feature (956)..(956): N = Adenine or Guanine or Cytosine or Thymine
  misc_feature (960)..(960): N = Adenine or Guanine or Cytosine or Thymine
  misc_feature (983)..(983): N = Adenine or Guanine or Cytosine or Thymine
gaaggataaatagggccgtagggccatcgttttgcattgccgtcgcgtggccgtgggttttcatcgacgaacgacgacgactctgccgcgcgcgtttaggcgcgtcgccgccggccacccggccggccacttcgacgcggctgtggtggtttaaaattctccggctcgccgcccacggcctcgctgcacgtagttgcaggtgagtgaggttcttcgattccccccaaacccggcccatctccgacgatccattatttgttcgtccatatgaaaatgcatctctttttgtacccagttgttacctacatgcagcttttactgcttcattcagttatggtcgtgtacatcttcagaaaaccagacatgttcaggcaagaccagtgggtatgcaaaaacaacagcaaagtggctcggcatgtcgacagccagctgctacttctacttctacatataaaccacgcgctggcatgaactccactccagcatggatagacgacatcgtcgcacaaatcacataccaggtcaaggcaccacgtttcacaaacctagtgaacacccaagaactattatgcaataccatcattttgctcgatagattttcctagcccagttgtttcatctgagcaagtgtctgtaggaactaaaacacagtagtatgctgtatatactgtcaagcatgaaacgcaaccagatcagctgcaggatttgcagcaccaagctctggccattaggtaggctgcatatcacaacacacaaagggaacatcaaactacatacactagctccgtcccctccagggacactgcaggttggcaggcccaaagntagcacagggtctcgcaacccacaaggtcataatcagtacaancatccatcggcaaggggaagcaagancnaatcccaggtaggtgnacgctggacagatccgtgctcannncgccgttccttgctccggncttntctggccgcttgtactagctccnctgttttgtttcgccaacatct

SEQ ID NO: 141 - DNA, 22 bp, Artificial Sequence
  SM3814 F primer
gataccgccgctatgttctttc

SEQ ID NO: 142 - DNA, 25 bp, Artificial Sequence
  SM3814 R primer
cgtgcttcattcacatcacatggat

SEQ ID NO: 143 - DNA, 16 bp, Artificial Sequence
  SM3814 FAM
tgttcttggagcagaa

SEQ ID NO: 144 - DNA, 17 bp, Artificial Sequence
  SM3814 TET
cctgttcttgtagcaga

SEQ ID NO: 145 - DNA, 1001 bp, Zea mays
tcaaagcgtactgggatggaaaagtgtagagtagaccggtacagacgccggagtaccacaatgacgctacagttcctacacgtcgaggtgcagtccagcctccacctgtagaggactagcgctactgccgctgccgcctgccagtgccagagaggcagagcgcagcctgtctgtgagggctccgctccggccgccagctttccaaggtgacaggtcctttctgtttctgactcctctgcgagcgcggggccctgcgcaatcagcgtctcggtccatatttttgattttattttcagactcgagaacacgcaaaggcaaaaggcagcaggagacaccgagatcgccggcaggcggcagccggacatatgcggcggcgccgccgctccatcctgtcgtgctgcttgcccgctcttcttccatttgtatcgtctgcagataccgccgctatgttctttctatagcaaatagagataacgagagatctttccagttcttctgctacaagaacaggaaactgtgtatccatgtgatgtgaatgaagcacgacaatttttttttttctgattctccaacaacggacgaaccagcttagatttccacttgctgttgtaacatgacaccacagggcgacacaggctaattacacacaaaagaaccccggagaagctaccctatccaccactacacaccttcagtccacaagaactctcatagtttactctactactgtaatgtaatctgtggctcacaccatccagatccatgttccctccttcctccgtcaccatctgacgatggtaagccaccagcagccaaaaacaaatgccaaaagatcacagagaacgcgcagaaactacttcgtgctaacctacctagcattacttcaagtttctccgtgttcttcttctgaacctgcttgcttgctattgtaggagggacaccaacccaagaaccgcagccgccattccccgaaccaccaccaggtaccggagcccaccgac

SEQ ID NO: 146 - DNA, 1001 bp, Zea mays
tcaaagcgtactgggatggaaaagtgtagagtagaccggtacagacgccggagtaccacaatgacgctacagttcctacacgtcgaggtgcagtccagcctccacctgtagaggactagcgctactgccgctgccgcctgccagtgccagagaggcagagcgcagcctgtctgtgagggctccgctccggccgccagctttccaaggtgacaggtcctttctgtttctgactcctctgcgagcgcggggccctgcgcaatcagcgtctcggtccatatttttgattttattttcagactcgagaacacgcaaaggcaaaaggcagcaggagacaccgagatcgccggcaggcggcagccggacatatgcggcggcgccgccgctccatcctgtcgtgctgcttgcccgctcttcttccatttgtatcgtctgcagataccgccgctatgttctttctatagcaaatagagataacgagagatctttccagttcttctgctccaagaacaggaaactgtgtatccatgtgatgtgaatgaagcacgacaatttttttttttctgattctccaacaacggacgaaccagcttagatttccacttgctgttgtaacatgacaccacagggcgacacaggctaattacacacaaaagaaccccggagaagctaccctatccaccactacacaccttcagtccacaagaactctcatagtttactctactactgtaatgtaatctgtggctcacaccatccagatccatgttccctccttcctccgtcaccatctgacgatggtaagccaccagcagccaaaaacaaatgccaaaagatcacagagaacgcgcagaaactacttcgtgctaacctacctagcattacttcaagtttctccgtgttcttcttctgaacctgcttgcttgctattgtaggagggacaccaacccaagaaccgcagccgccattccccgaaccaccaccaggtaccggagcccaccgac

SEQ ID NO: 147 - DNA, 22 bp, Artificial Sequence
  SM3362 F primer
ccgcgtctttgttgtagcattc

SEQ ID NO: 148 - DNA, 23 bp, Artificial Sequence
  SM3362 R primer
tcaggaaacatcagtgcccatac

SEQ ID NO: 149 - DNA, 19 bp, Artificial Sequence
  SM3362 FAM
actagttgcctaccctatc

SEQ ID NO: 150 - DNA, 19 bp, Artificial Sequence
  SM3362 TET
tagttgcctactctatcag

SEQ ID NO: 151 - DNA, 1001 bp, Zea mays
gcaagatgacaaatatttgtttttcatggcattgatgtagcctacaagatggaactttgactagaaatatctataaaaaaacattgtttcgaatataaagagttactccatctgtcctggaatgagaagtgtattttgatcaaagaaaagtcatacaaaattgatttgaccccttagtttctgttgcaatatgttatcatttgccaaacagtcaataccatctctcagaggcaccttgaatgcacatacattatggttcttttttcccatccattgcagatatgactaaaatgaagatcgacggcgacaataactttggggagcagaagagccatcaccgctgcaggcgcaagaagcatgatgctagggttttggattccttgagttgaagatttttgttgtgctagaagtggtgaagcgtctaagcatgtgttgtattgtagtgttccttcactagcagcagctgttatccgcgtctttgttgtagcattcactgatagagtaggcaactagttaccagtgtttctgactttttgtatgggcactgatgtttcctgatagtatagttccctagtttctgggctagtttttttttccatgagtttatctgtgaaggcttcaagagtagcgaatgcgctaagaacaaatgtgtcgcctttttagttgttttttacacttgttagcactatataatggttgtttgagttggatgcgtgattgatacagcagcatcagtcagctgatccctaaaataaggttgtttggtttaaggtcaggggttgggacatgactatcctagtgttgtcccagttatcactcaaaattggatcctagtgttgtcccagttatcactcaaaattggagggacgagagaggacgccagggaacgtccctgtcctggttgtcaagttatcccttaaaattaggcagcagtgattaacactgccatcgattacagcgcaggctaatttttacacgaattatatgtgttatatcctgct

SEQ ID NO: 152 - DNA, 1001 bp, Zea mays
gcaagatgacaaatatttgtttttcatggcattgatgtagcctacaagatggaactttgactagaaatatctataaaaaaacattgtttcgaatataaagagttactccatctgtcctggaatgagaagtgtattttgatcaaagaaaagtcatacaaaattgatttgaccccttagtttctgttgcaatatgttatcatttgccaaacagtcaataccatctctcagaggcaccttgaatgcacatacattatggttcttttttcccatccattgcagatatgactaaaatgaagatcgacggcgacaataactttggggagcagaagagccatcaccgctgcaggcgcaagaagcatgatgctagggttttggattccttgagttgaagatttttgttgtgctagaagtggtgaagcgtctaagcatgtgttgtattgtagtgttccttcactagcagcagctgttatccgcgtctttgttgtagcattcactgatagggtaggcaactagttaccagtgtttctgactttttgtatgggcactgatgtttcctgatagtatagttccctagtttctgggctagtttttttttccatgagtttatctgtgaaggcttcaagagtagcgaatgcgctaagaacaaatgtgtcgcctttttagttgttttttacacttgttagcactatataatggttgtttgagttggatgcgtgattgatacagcagcatcagtcagctgatccctaaaataaggttgtttggtttaaggtcaggggttgggacatgactatcctagtgttgtcccagttatcactcaaaattggatcctagtgttgtcccagttatcactcaaaattggagggacgagagaggacgccagggaacgtccctgtcctggttgtcaagttatcccttaaaattaggcagcagtgattaacactgccatcgattacagcgcaggctaatttttacacgaattatatgtgttatatcctgct

SEQ ID NO: 153 - DNA, 23 bp, Artificial Sequence
  SM0634AQ F primer
gcatgatatcctccatggtaggg

SEQ ID NO: 154 - DNA, 24 bp, Artificial Sequence
  SM0634AQ R primer
gttttcgcaagcaatgagaatggt

SEQ ID NO: 155 - DNA, 20 bp, Artificial Sequence
  SM0634AQ FAM
aaagtattccatgtccttac

SEQ ID NO: 156 - DNA, 22 bp, Artificial Sequence
  SM0634AQ TET
agaaagtattccatctccttac

SEQ ID NO: 157 - DNA, 171 bp, Zea mays
ttgcatgatatcctccatggtagggagaaagtattccatctccttacataagctcagtgtttttaccattctcattgcttgcgaaaacatttatctacttccataataagmtactwtttgcagttgctcccatgctwactastatgaayyggttgctgatgcctaattwaa

SEQ ID NO: 158 - DNA, 171 bp, Zea mays
ttgcatgatatcctccatggtagggagaaagtattccatgtccttacataagctcagtgtttttaccattctcattgcttgcgaaaacatttatctacttccataataagmtactwtttgcagttgctcccatgctwactastatgaayyggttgctgatgcctaattwaa

SEQ ID NO: 159 - DNA, 21 bp, Artificial Sequence
  SM4586 F primer
cagagccactcgtcaggagat

SEQ ID NO: 160 - DNA, 19 bp, Artificial Sequence
  SM4586 R primer
ggtgtactcgccggagtta

SEQ ID NO: 161 - DNA, 14 bp, Artificial Sequence
  SM4586 FAM
agatgcggtggcgc

SEQ ID NO: 162 - DNA, 16 bp, Artificial Sequence
  SM4586 TET
taagatgtggtggcgc

SEQ ID NO: 163 - DNA, 1001 bp, Zea mays
tgcatgactgtctgaggtccttaggacaaggacatatgtaggtgggcctaataccttgggaggttcagtgacaccctagatggcggtcataaataccaaagagcctagggtcagtcagacaaaattttagggctaatttgtaacattcttttaatatatttggacaatgggtttatagtaacaaaaccgaggggatcttatgtcaaattgcatgatgaaagggtattggatgattctagtcacccgatctgaaacctgggatccatattagatcacctctagccaaaccggtgcgtgatccaaatcatccgatcgacaatcgatgatgaggatttaaaataccttgctttgcctagagtcgtccaacgagatccgagggcgcagatgcaatagccaaaggggtacgcatggggccgcaccagagccactcgtcaggagatcaacggctgcccaagctcttgctggccacccagcgcagaacatagtggctgtgcgccaccacatcttataactccggcgagtacaccaatcaggtgccctaatgccctacgcgcctacctaattgcatttacacgacgagagggcgacaagaaatcaatggagatggcccatactgtgggaggggtgcgaaggttcgtgttcccggtgaagggtggtgctgcggtggagaactcgcgtcggtgtgaaatttacccagcaggttccacgactctcgtggtcaagccttggcatgcaccagatgtgacggaacctcccaagggattaggcccacctacagttctccttgtcctaaggactttggacaaccctgtagatgcacataatcactcgacaagttcggtaactgtatccttatcatttcgcccaagagcgcttcacccatcacgcagatattacatcacatcggaggaaagaataagcggaagcagattacaataacttaatttacattcatcaaatatataaagagagtattattattataacaataccagggtatt

SEQ ID NO: 164 - DNA, 1001 bp, Zea mays
tgcatgactgtctgaggtccttaggacaaggacatatgtaggtgggcctaataccttgggaggttcagtgacaccctagatggcggtcataaataccaaagagcctagggtcagtcagacaaaattttagggctaatttgtaacattcttttaatatatttggacaatgggtttatagtaacaaaaccgaggggatcttatgtcaaattgcatgatgaaagggtattggatgattctagtcacccgatctgaaacctgggatccatattagatcacctctagccaaaccggtgcgtgatccaaatcatccgatcgacaatcgatgatgaggatttaaaataccttgctttgcctagagtcgtccaacgagatccgagggcgcagatgcaatagccaaaggggtacgcatggggccgcaccagagccactcgtcaggagatcaacggctgcccaagctcttgctggccacccagcgcagaacatagtggctgtgcgccaccgcatcttataactccggcgagtacaccaatcaggtgccctaatgccctacgcgcctacctaattgcatttacacgacgagagggcgacaagaaatcaatggagatggcccatactgtgggaggggtgcgaaggttcgtgttcccggtgaagggtggtgctgcggtggagaactcgcgtcggtgtgaaatttacccagcaggttccacgactctcgtggtcaagccttggcatgcaccagatgtgacggaacctcccaagggattaggcccacctacagttctccttgtcctaaggactttggacaaccctgtagatgcacataatcactcgacaagttcggtaactgtatccttatcatttcgcccaagagcgcttcacccatcacgcagatattacatcacatcggaggaaagaataagcggaagcagattacaataacttaatttacattcatcaaatatataaagagagtattattattataacaataccagggtatt

SEQ ID NO: 165 - DNA, 23 bp, Artificial Sequence
  gRNA 1 (DMP)
ctccctgggcacctcgacgccga

SEQ ID NO: 166 - DNA, 20 bp, Artificial Sequence
  gRNA 2 (DMP)
ggccgggagtagcatctcga

SEQ ID NO: 167 - DNA, 20 bp, Artificial Sequence
  gRNA 3 (DMP)
actacggcttcgtcacgccg

SEQ ID NO: 168 - DNA, 23 bp, Artificial Sequence
  gRNA 4 (MATL)
catgcagaactgcccgcgcatct

SEQ ID NO: 169 - DNA, 20 bp, Artificial Sequence
  gRNA 5 (MATL)
gggtcaacgtggagacaggg

SEQ ID NO: 170 - DNA, 20 bp, Artificial Sequence
  gRNA 6 (MATL)
cttcctggaggccaggctgc

SEQ ID NO: 171 - DNA, 17462 bp, Artificial Sequence
  Vector 26258
  misc_feature (4)..(143): bNRB
  promoter (222)..(2018): prSoUbi4-04
  5′UTR (596)..(660): u5SoUbi4-02
  Intron (661)..(2018): iSoUbi4-02
  gene (2037)..(6318): LbCas12a-27
  misc_signal (2040)..(2060): SV40NLS-06
  Intron (2672)..(3080): iAtBAF60-01
  misc-signal (6250)..(6270): xSV40NLS-04
  misc-signal (6295)..(6315): xSV40NLS-07
  terminator (6327)..(6579): tNOS-05-01
  promoter (6607)..(7122): prOsU6-01
  misc_RNA (7124)..(7144): rLbCrRNA-01
  misc_RNA (7124)..(7167): rLbgRNACas12aZmO2-01
  misc_RNA (7168)..(7188): rLbCrRNA-01
  misc_RNA (7168)..(7211): rLbgRNACas12aZmYellow1-01
  misc_RNA (7212)..(7232): rLbCrRNA-01
  misc_RNA (7212)..(7255): rLbgRNACas12aZmWaxy1-01
  promoter (7271)..(7786): prOsU6-01
  misc_RNA (7788)..(7808): rLbCrRNA-01
  misc_RNA (7788)..(7830): rLbgRNACas12aZmUBL-01
  misc_RNA (7831)..(7851): rLbCrRNA-01
  misc_RNA (7831)..(7873): rLbgRNACas12aZmUPL3-01
  promoter (7889)..(9881): prUbi1-18
  Intron (8872)..(9881): iUbi1-07
  gene (9889)..(11064): cPMI-14
  terminator (11077)..(12111): tUbi1-06
  misc_feature (12194)..(12323): bNLB-05
  misc_feature (12229)..(12253): bNLB-01-01
  gene (12603)..(13391): cSpec-03
  promoter (13486)..(13616): prVirG-02
  gene (14446)..(15519): cRepA-05
  rep_origin (15562)..(15966): oVS1-03
  rep_origin (16644)..(17450): oCOLE-06
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaacccgggggcgcgccgggacccgaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacgagaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtaggagaaattcatcgaagctaagcgaatcctcgcgatcctctcaaggtactgcgagttttcgatccccctctcgacccctcgtatgtttgtgtttgtcgtagcgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatgatggttggaccggttggttcgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaattgtttggtcctttactcatgttgcaattatgcaatttagtttagattgtttgttccactcatctaggctgtaaaagggacactgcttagattgctgtttaatctttttagtagattatattatattggtaacttattacccctattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatgccacaatttttagtcacttaacccatgcagattgaactggtccctgcatgttttgctaaattgttctattctgattagaccatatatcatgtatttttttttggtaatggttctcttattttaaatgctatatagttctggtacttgttagaaagatctgcttcatagtttagttgcctatccctcgaattaggatgctgagcagctgatcctatagctttgtttcatgtatcaattcttttgtgttcaacagtcagtttttgttagattcattgtaacttatggtcgcttactcttctggtcctcaatgcttgcagggatcccctaaatagaccatgccgaagaagaagcgcaaggtcgggggcgggggctcaggcgggggcgggagcggcggcgggggctctgggggcggcggcagcggcgggggcggcagcgggggcggcgggtcgatgagcaagctggagaagttcacgaactgctactccctcagcaagaccctgaggttcaaggcgatcccggtcggcaagacccaggagaacatcgacaacaagcggctgctggtggaggacgagaagagggctgaggactacaagggcgtgaagaagctcctggaccgctactacctgtccttcatcaacgacgtgctccacagcatcaagctcaagaacctgaacaactacatcagcctcttcaggaagaagacgcgcaccgagaaggagaacaaggagctcgagaacctggagatcaacctgaggaaggagatcgccaaggcgttcaagggcaacgagggctacaagtccctcttcaagaaggacatcatcgagacgatcctcccggagttcctggacgacaaggacgagatcgccctggtcaactccttcaacggcttcaccacggcgttcaccggcttcttcgacaaccgcgagaacatgttcagcgaggaggccaagtccacgagcatcgcgttcaggtaccaagctgcgaatcttcgtttttttaaggaattctcgatctttatggtgtataggctctgggttttctgttttttgtatctcttaggattttgtaaattccagatctttctatggccacttagtagtatatttcaaaaattctccaatcgagttcttcattcgcattttcagtcattttctcttcgacgttgtttttaagcctgggtattactcctatttagttgaactctgcagcaatcttagaaaattagggttttgaggtttcgatttctctaggtaaccgatctattgcattcatctgaatttctgcatatatgtcttagatttctgataagcttacgatacgttaggtgtaattgaagtttatttttcaagagtgttattttttgtttctgaatttttcaggtgcatcaacgagaacctcacccgctacatctccaacatggacatcttcgagaaggtcgacgcgatcttcgacaagcacgaggtgcaggagatcaaggagaagatcctgaacagcgactacgacgtcgaggacttcttcgagggcgagttcttcaacttcgtcctcacgcaggagggcatcgacgtgtacaacgccatcatcggtggcttcgtgaccgagtccggcgagaagatcaagggcctgaacgagtacatcaacctctacaaccagaagaccaagcagaagctgccgaagttcaagcccctgtacaagcaggtgctctccgacagggagtccctcagcttctacggcgagggctacacgagcgacgaggaggtcctggaggtgttccgcaacaccctcaacaagaacagcgagatcttctccagcatcaagaagctcgagaagctgttcaagaacttcgacgagtactccagcgccggcatcttcgtcaagaacggcccggcgatctccacgatcagcaaggacatcttcggcgagtggaacgtgatccgcgacaagtggaacgccgagtacgacgacatccacctcaagaagaaggcggtggtcaccgagaagtacgaggacgacaggcgcaagtccttcaagaagatcggctccttcagcctcgagcagctgcaggagtacgccgacgcggacctgagcgtggtcgagaagctcaaggagatcatcatccagaaggtcgacgagatctacaaggtgtacggctccagcgagaagctcttcgacgcggacttcgtcctcgagaagtccctgaagaagaacgacgccgtggtcgcgatcatgaaggacctcctggactccgtgaagagcttcgagaattacatcaaggccttcttcggcgagggcaaggagacgaacagggacgagtccttctacggcgacttcgtcctggcctacgacatcctcctgaaggtggaccacatctacgacgcgatccgcaactacgtgacccagaagccgtacagcaaggacaagttcaagctctacttccagaacccccagttcatgggcggctgggacaaggacaaggagacggactacagggcgaccatcctgcgctacggcagcaagtactacctcgccatcatggacaagaagtacgcgaagtgcctgcagaagatcgacaaggacgacgtcaacggcaactacgagaagatcaactacaagctcctgccgggccccaacaagatgctcccgaaggtgttcttctccaagaagtggatggcctactacaaccccagcgaggacatccagaagatctacaagaacggcacgttcaagaagggcgacatgttcaacctgaacgactgccacaagctcatcgacttcttcaaggactccatcagccgctacccgaagtggtccaacgcctacgacttcaacttcagcgagaccgagaagtacaaggacatcgcgggcttctaccgcgaggtcgaggagcagggctacaaggtgtccttcgagtccgccagcaagaaggaggtcgacaagctggtggaggagggcaagctctacatgttccagatctacaacaaggacttctccgacaagagccacggcacgcccaacctgcacaccatgtacttcaagctcctgttcgacgagaacaaccacggccagatcaggctgtccggcggcgccgagctcttcatgaggagggcgagcctgaagaaggaggagctggtggtccaccccgctaacagcccaatcgcgaacaagaacccggacaaccccaagaagaccacgaccctgtcctacgacgtgtacaaggacaagaggttcagcgaggaccagtacgagctccacatcccgatcgcgatcaacaagtgccccaagaacatcttcaagatcaacaccgaggtccgcgtgctcctgaagcacgacgacaacccctacgtgatcggcatcgacaggggcgagaggaacctcctgtacatcgtggtcgtggacggcaagggcaacatcgtggagcagtactccctcaacgagatcatcaacaacttcaacggcatcaggatcaagacggactaccacagcctcctggacaagaaggagaaggagaggttcgaggcccgccagaactggacctccatcgagaacatcaaggagctgaaggcgggctacatcagccaggtcgtgcacaagatctgcgagctcgtcgagaagtacgacgccgtgatcgccctcgaggacctgaactccggcttcaagaacagccgcgtcaaggtggagaagcaggtctaccagaagttcgagaagatgctcatcgacaagctgaactacatggtggacaagaagtccaacccctgcgctacgggcggcgcgctgaagggctaccagatcaccaacaagttcgagagcttcaagtccatgagcactcagaacggcttcatcttctacatcccggcgtggctcacgtccaagatcgaccccagcaccggcttcgtcaacctcctgaagacgaagtacacctccatcgccgacagcaagaagttcatctccagcttcgaccgcatcatgtatgtgccggaggaggacctgttcgagttcgccctcgactacaagaacttctcccgcacggacgcggactacatcaagaagtggaagctgtacagctacggcaaccgcatccgcatcttcaggaaccccaagaagaacaacgtcttcgactgggaggaggtgtgcctgacctccgcgtacaaggagctcttcaacaagtacggcatcaactaccagcagggcgacatcagggctctcctgtgcgagcagagcgacaaggccttctactccagcttcatggcgctgatgtccctcatgctgcagatgaggaactcgatcaccggcaggacggacgtggacttcctcatctccccggtgaagaacagcgacggcatcttctacgactccaggaactacgaggcccaggagaacgcgatcctcccaaagaacgcggacgccaacggcgcctacaacatcgccaggaaggtcctctgggctatcggccagttcaagaaggcggaggacgagaagctggacaaggtgaagatcgccatcagcaacaaggagtggctcgagtacgcccagacctcggtcaagcacggcagcccgaagaagaagcgcaaggtgtccggcggcagctccggcggcagcccgaagaagaagcgcaaagtgtgattaattaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcttcgaacgcgttaactagctaggaatttttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgtaatttctactaagtgtagatctgtatctcgagcgtctggctgataatttctactaagtgtagatctatcttatcctaaagatggtggtaatttctactaagtgtagatgggaaagaccgaggagaagatcttttttttttcctaggtttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgtaatttctactaagtgtagatggaaggaaaaggtatctgaaggtaatttctactaagtgtagatggagggaaaaggtgtctgaggcttttttttcggaccgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagctaaaccatgcagaagctgatcaacagcgtgcagaactacgcctggggcagcaagaccgccctgaccgagctgtacggcatggagaaccccagcagccagccaatggccgagctgtggatgggcgcccaccccaaaagctcaagccgcgtgcagaacgccgccggcgatatcgttagcctgcgcgacgtgatcgagagcgacaagagcaccctgctgggcgaggccgtggccaagcgcttcggcgagctgcccttcctgttcaaggtgctgtgcgccgctcagcccctgagcatccaggtgcaccctaacaagcacaacagcgagatcggcttcgccaaggagaacgccgccggcatcccaatggacgccgccgagcgcaactacaaggaccccaaccacaagcccgagctggtgttcgccctgacccccttcctggccatgaacgccttccgcgagttcagcgagatcgttagcctgctgcagcccgtggccggcgcccaccccgctatcgcccacttccttcagcagcccgacgccgagcgcctgagcgagctgttcgccagcctgctgaacatgcagggtgaggagaagtcacgcgccctggccatcctgaagagcgccctggacagccagcagggcgagccctggcagacaatccgcctgatcagcgagttctaccccgaggatagcggcctgttcagccccctgctgctgaacgtggtgaagctgaaccccggcgaggccatgttcctgttcgccgagactccccacgcctacctgcagggcgtggccctggaggtgatggccaacagcgacaacgtgctgcgcgccggcctgacccccaagtacatcgacatccccgagctggtggccaacgtgaagttcgaggctaagcccgccaaccagctgctgacccagcccgtgaagcagggcgccgagctggacttccctatccccgttgacgacttcgccttcagcctgcacgacctgagcgacaaggagactactatcagccagcagagcgccgcgatcctgttctgcgtggagggcgacgccaccctgtggaagggcagccagcagctgcagctgaagcccggcgagagcgcctttatcgccgccaacgagagccccgtgaccgtgaagggccacggccgcctggcccgcgtgtacaacaagctgtgataggattacctagtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctgctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcctgcaggcccgggggcgcgccctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgtgaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgccgatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggaaacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcgggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgatgagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 172 - DNA, 23 bp, Artificial Sequence
  Opaque2 gRNA target sequence
ctgtatctcgagcgtctggctga

SEQ ID NO: 173 - DNA, 23 bp, Artificial Sequence
  Waxy1 gRNA target sequence
gggaaagaccgaggagaagatct

SEQ ID NO: 174 - DNA, 23 bp, Artificial Sequence
  Yellow Endosperm1 gRNA target sequence
ctatcttatcctaaagatggtgg

SEQ ID NO: 175 - DNA, 22 bp, Artificial Sequence
  E3 ubiquitin ligase2 gRNA target sequence
ggagggaaaaggtgtctgaggc

SEQ ID NO: 176 - DNA, 22 bp, Artificial Sequence
  putatitive ubiquitin-protein ligase gRNA target sequence
ggaaggaaaaggtatctgaagg

SEQ ID NO: 177 - DNA, 19587 bp, Artificial Sequence
  Vector 24288
  misc_feature (4)..(143): bNRB-04
  misc_feature (101)..(125): bNRB-01-01
  promoter (205)..(601): prCMP-04
  5′UTR (205)..(601)
  gene (638)..(1417): cDsRed2Nu-03
  misc_signal (1325)..(1414): xSV40NLS-02
  terminator (1418)..(1687): t35S-12
  misc_signal (1659)..(1661): Poly\A\site
  promoter (1694)..(2296): prRab17-05
  gene (2306)..(3337): cCRE-01
  terminator (3343)..(3660): tPI-15
  promoter (3675)..(3863): prNOS-05-01
  gene (3872)..(4528): cSbWUS-01
  terminator (4538)..(4790): tNOS-05-01
  promoter (4810)..(6802): prUbi1-18
  Intron (5793)..(6802): iUbi1-07
  gene (6820)..(8559): cBnBBM1-02
  terminator (8569)..(9603): tUbi1-04
  gene (9654)..(10343): cAmCyan-03
  terminator (10351)..(10603): tNOS-05-01
  promoter (10611)..(12795): prAct1-09
  Intron (12313)..(12774): iAct1-03
  gene (12807)..(13982): cPMI-01
  terminator (13993)..(14245): tNOS-05-01
  misc_feature (14319)..(14448): bNLB-05
  misc_feature (14354)..(14378): bNLB-01-01
  gene (14728)..(15516): cSpec-03
  promoter (15611)..(15741): prVirG-02
  gene (15816)..(16541): cVirG-06
  gene (16571)..(17644): cRepA-10
  rep_origin (17687)..(18091): oVS1-02
  rep_origin (18769)..(19575): oCOLE-06
attcctgtggttggcatgcacatacaaatggacgaacggataaaccttttcacgcccttttaaatatccgattattctaataaacgctcttttctcttaggtttacccgccaatatatcctgtcaaacactgatagtttaaactggcactagcctaacggtgttgactaactaggccgcttccctaattagctaaatttaaatctggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagataacttcgtatagcatacattatacgaagttatatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacaatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgagatctcgagctgatccaaaaaagaagagaaaggtagatccaaaaaagaagagaaaggtagatccaaaaaagaagagaaaggtaggctccaccggatctagataatccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcacgtgttgagcatataagaaacccttagtatgtatttgtatttgtaaaatacttctatcaataaaatttctaattcctaaaaccaaaatccagtactaaaatccacctaggctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcatctgatcagcgatggccaatcttcttactgttcaccagaatttgccagctctgcctgtcgacgccacttcggacgaggttcgcaagaatctcatggacatgttccgcgacaggcaggccttcagcgagcacacctggaagatgctgctgtcggtgtgcaggtcctgggctgcgtggtgcaagctgaataataggaagtggttccccgccgagccggaggacgtgcgcgactacctcctgtacctgcaggctcgcggcctcgccgtcaagacgatccagcagcacttgggccagctgaacatgctgcacaggcgcagcggcctgccgaggccaagcgacagcaacgccgtgtccttggtcatgcgcaggattcgcaaggagaacgtggacgcgggcgagcgcgccaagcaggccctggccttcgagcgcaccgacttcgaccaggtcaggagcctgatggagaacagcgacaggtgccaggacatcaggaacctggccttcctcggcatcgcgtacaatacactcctgaggatcgcggagatcgcccgcatcagggtcaaggacatctcacgcacggacggtggcaggatgctgatacatatcggcaggacgaagaccctcgtgagcacggcgggcgtggagaaggcgctctccctgggcgtgacgaagctggtcgagcggtggatctccgtgagcggcgtggcggacgacccgaataattacctcttctgccgcgtgaggaagaacggcgtggcggccccatcggccaccagccagctgtcgaccagggctctggagggcatcttcgaggcgacccacaggctgatctacggcgctaaggacgactcgggccagcgctacctcgcttggtcgggccactcggccagggtgggcgcggcccgcgacatggccagggcgggcgtctccatcccggagatcatgcaggctggcggctggacgaacgtgaacatcgtcatgaactacattaggaatctggactcggagactggggcgatggttcggctgctggaggacggcgattaattcgaagacttgtccatcttctggattggccaacttaattaatgtatgaaataaaaggatgcacacatagtgacatgctaatcactataatgtgggcatcaaagttgtgtgttatgtgtaattgctagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggcctcgagaccggtacctttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctgatcaaaaatggaggcgctgagcgggcgggtaggcgtcaagtgcgggcggtggaaccctacggcggagcaggtgaaggtcctgacggagctgttccgcgcggggctgcgcacgcccagcacggagcagatccagcgcatctccacccacctcagcgccttcggcaaggtggagagcaagaacgtcttctactggttccagaaccacaaggcccgcgagcgccaccaccacaagaagcgccgccgcggcgcgtcctcccccgacagtggcagcggctccggcagcggcagcaacgaggaagacggcggccgtgctgctgccgcctcgcacgacgccgaggccgacgtcgacctcgtgctgcagccgccagagagcaagcgggaggccagaagctacgcccaccatcatcaccggctggccgtgacatgctacgtcagggacgtggtggagcagcaggaggccacgtgggagcggccgacgcgcgaggtggagacgctggagctgttccctctcaagtcgtacgtggacctggaggctgcggagaaggtccggtatgtcaggggcagcgccgccagcgagcagtgcagggagttctccttcttcgacgtctccggcggccgcgatccgccacttgagctgaggctctgcagcttcggtccctactaagaattctaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtacctacgtacggacccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgccgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatagttagggcccggtagttctacttctgttcatgtttgtgttagatccgtgtttgtgttagatccgtgctgttagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatctaactagttaaaatggctaataattggcttggtttctctctttcaccatacgagcagaatcatcatcggaaggacgtttactcatccaccactacgaccgtggtcgacgtggcgggcgagtactgctacgacccgacggcggcctcggacgagtccagcgcgatccagacgtccttccccagcccattcggcgtggtcgtggacgctttcaccagggacaacaactcccacagcagggactgggacatcaacggctgcgcctgcaacaacatccataacgacgagcaggacggcccgaagctcgagaacttcctgggccgcaccacgaccatctacaatactaacgagaacgtgggcgacggcagcggcagcgggtgctacggtggtggcgacggcagcggcggctccctcggcctgagcatgatcaagacgtggctgcgcaaccagcccgtggacaacgtcgacaaccaggagaacggcaacgcggccaagggcctctccctgagcatgaactccagcacctcctgcgacaacaacaacgattccaacaacaacgtggtcgcccagggcaagacgatcgacgactccgtggaggcgaccccgaagaagacgatcgagtccttcggccagcgcacttctatctacaggggcgtcacccgccacaggtggaccggccgctacgaggcgcacctctgggataacagctgcaagagggagggccagacccgcaagggcaggcaggtgtacctgggcggctacgacaaggaggagaaggcggccagggcttacgacctcgcggccctgaagtactggggcacgaccacgaccacgaacttcccgatgtccgagtacgagaaggaggtggaggagatgaagcacatgacccgccaggagtatgtggctagcctcaggaggaagtccagcggcttctccaggggcgcgagcatctaccgcggcgtgacgaggcaccaccagcacggcaggtggcaggctcgcatcggcagggtcgcgggcaacaaggacctctacctgggcaccttcggcacgcaggaggaggctgccgaggcttacgacatcgctgcgatcaagttccgcggcctcaccgcggtgacgaacttcgacatgaacaggtacaacgtcaaggccatcctcgagagccccagcctgccaatcggctccgctgccaagaggctgaaggaggccaacaggccggtgccctccatgatgatgatcagcaacaacgtctccgagtcggagaactcggcttcgggctggcagaacgcggccgtgcagcaccaccagggcgtcgacctctccctcctccaccagcaccaggagaggtacaacggctactactacaacggcggcaacctgtccagcgagtcggctagggcctgcttcaagcaggaggacgaccagcaccacttcctctccaatacacagagcctgatgacgaacatcgaccaccagtccagcgtgtccgacgacagcgtgacggtctgcggcaacgtggtgggctacggcggctaccagggcttcgcggccccagtcaactgcgacgcctacgccgcgagcgagttcgactacaacgcccgcaaccactactacttcgcgcagcagcagcagacccagcagtccccgggcggcgacttcccggctgcgatgacgaacaacgtgggcagcaacatgtactaccacggcgagggcggcggcgaggttgctccgactttcactgtttggaacgataattaaacgcgttaagtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagctacgtatttataacttcgtatagcatacattatacgaagttattaaaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgattatatagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaccctagcttgcatgcctgcagcccatccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgcgctcctcccccctccccctccgccgccctagcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtcgatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaagctggctgacgccggatccctagatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaatcaagagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccggaccgcgatcgctaattagctaacggccaggatcgccgcgtgagcctttagcaactagctagattaattaacgcaatctgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgtgaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgccgatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggaaacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcgggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 178 - DNA, 657 bp, Sorghum bicolor
atggaggcgctgagcgggcgggtaggcgtcaagtgcgggcggtggaaccctacggcggagcaggtgaaggtcctgacggagctgttccgcgcggggctgcgcacgcccagcacggagcagatccagcgcatctccacccacctcagcgccttcggcaaggtggagagcaagaacgtcttctactggttccagaaccacaaggcccgcgagcgccaccaccacaagaagcgccgccgcggcgcgtcctcccccgacagtggcagcggctccggcagcggcagcaacgaggaagacggcggccgtgctgctgccgcctcgcacgacgccgaggccgacgtcgacctcgtgctgcagccgccagagagcaagcgggaggccagaagctacgcccaccatcatcaccggctggccgtgacatgctacgtcagggacgtggtggagcagcaggaggccacgtgggagcggccgacgcgcgaggtggagacgctggagctgttccctctcaagtcgtacgtggacctggaggctgcggagaaggtccggtatgtcaggggcagcgccgccagcgagcagtgcagggagttctccttcttcgacgtctccggcggccgcgatccgccacttgagctgaggctctgcagcttcggtccctactaa

SEQ ID NO: 179 - DNA, 1740 bp, Brassica napus
atggctaataattggcttggtttctctctttcaccatacgagcagaatcatcatcggaaggacgtttactcatccaccactacgaccgtggtcgacgtggcgggcgagtactgctacgacccgacggcggcctcggacgagtccagcgcgatccagacgtccttccccagcccattcggcgtggtcgtggacgctttcaccagggacaacaactcccacagcagggactgggacatcaacggctgcgcctgcaacaacatccataacgacgagcaggacggcccgaagctcgagaacttcctgggccgcaccacgaccatctacaatactaacgagaacgtgggcgacggcagcggcagcgggtgctacggtggtggcgacggcagcggcggctccctcggcctgagcatgatcaagacgtggctgcgcaaccagcccgtggacaacgtcgacaaccaggagaacggcaacgcggccaagggcctctccctgagcatgaactccagcacctcctgcgacaacaacaacgattccaacaacaacgtggtcgcccagggcaagacgatcgacgactccgtggaggcgaccccgaagaagacgatcgagtccttcggccagcgcacttctatctacaggggcgtcacccgccacaggtggaccggccgctacgaggcgcacctctgggataacagctgcaagagggagggccagacccgcaagggcaggcaggtgtacctgggcggctacgacaaggaggagaaggcggccagggcttacgacctcgcggccctgaagtactggggcacgaccacgaccacgaacttcccgatgtccgagtacgagaaggaggtggaggagatgaagcacatgacccgccaggagtatgtggctagcctcaggaggaagtccagcggcttctccaggggcgcgagcatctaccgcggcgtgacgaggcaccaccagcacggcaggtggcaggctcgcatcggcagggtcgcgggcaacaaggacctctacctgggcaccttcggcacgcaggaggaggctgccgaggcttacgacatcgctgcgatcaagttccgcggcctcaccgcggtgacgaacttcgacatgaacaggtacaacgtcaaggccatcctcgagagccccagcctgccaatcggctccgctgccaagaggctgaaggaggccaacaggccggtgccctccatgatgatgatcagcaacaacgtctccgagtcggagaactcggcttcgggctggcagaacgcggccgtgcagcaccaccagggcgtcgacctctccctcctccaccagcaccaggagaggtacaacggctactactacaacggcggcaacctgtccagcgagtcggctagggcctgcttcaagcaggaggacgaccagcaccacttcctctccaatacacagagcctgatgacgaacatcgaccaccagtccagcgtgtccgacgacagcgtgacggtctgcggcaacgtggtgggctacggcggctaccagggcttcgcggccccagtcaactgcgacgcctacgccgcgagcgagttcgactacaacgcccgcaaccactactacttcgcgcagcagcagcagacccagcagtccccgggcggcgacttcccggctgcgatgacgaacaacgtgggcagcaacatgtactaccacggcgagggcggcggcgaggttgctccgactttcactgtttggaacgataattaa

SEQ ID NO: 180 - DNA, 13283 bp, Artificial Sequence
  Vector 25072
  misc_feature (4)..(259): bNRB-05
  misc_feature (101)..(125): bNRB-01-01
  promoter (349)..(2342): prUbi1-44
  Intron (1333)..(2342): iUbi1-30
  gene (2367)..(3002): cBdWOX5/7-v1
  terminator (3011)..(4045): tUbi1-04
  promoter (4070)..(6062): prUbi1-10
  Intron (5053)..(6062): iUbi1-02-01
  gene (6071)..(7684): cPPO-04
  terminator (7710)..(7962): tNOS-05-01
  misc_feature (8015)..(8144): bNLB-03
  misc_feature (8050)..(8074): bNLB-01-01
  gene (8424)..(9212): cSpec-03
  gene (9512)..(10237): cVirG-01
  gene (10267)..(11340): cRepA-01
  rep_origin (11383)..(11787): oVS1-02
  rep_origin (12465)..(13271): oCOLE-06
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcagggaccccggaccctaattagctaaggtacctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcagggatcttcgaataacctagcagcgatggaggtgctgagcgggagggtaggggtgaagtgcgggcggtggaacccgacggcggagcaggtgaaggtgctgacggagcttttccgggcggggctgcggacgccgagcacggagcagatccagcggatctccacccacctcggcgccttcggcaaggtggagagcaagaacgtcttctactggttccagaaccacaaggcccgcgagcgccaccaccacaagaagcgccgccgcgtcgcctcctcctcctccgacaacagcagcgccagcaacaacaacgacgaagcagcagatcacggccgcagtagtgcccgcgaggacctcctgctgcagcctcccgagagcaagcgcgaggccagaagctacaaccaccaccgccggccgatcatgacatatgtaggctatgtgagggacgaggtggagcaggaggtggtgatgtgggagcggccgacgagggaggtggagacgctcgagctgttcccgctcaaggcagcctacgacctcgaggcggcggacaggctccggtacgtgaggggcgccggcgagcagcagtgcagggagatctccttcttcgacgtcgccaacggacgggatccgccgctggagctcaggctctgcagcttcgatatctagttaattaagtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctccaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaacgtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacttagtttggtttaatggaccggttctcctagcttgttctactcaaaactgttgttgatgcgaataagttgtgatggttgatctctggattttgttttgctctcaatagtggacgagattagatagcggaccgggtaccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatcccgatggaactctccctcctccgcccgaccacccagtccctcctcccgtccttctccaagccgaacctccgcctcaacgtgtacaagccgctccgcctccgctgctccgtggccggcggcccgaccgtgggctcctccaagatcgagggcggcggcggcaccaccatcaccaccgactgcgtgatcgtgggcggcggcatctccggcctctgcatcgcccaggccctcgccaccaagcacccggacgccgccccgaacctcatcgtgaccgaggccaaggaccgcgtgggcggcaacatcatcacccgcgaggagaacggcttcctctgggaggagggcccgaactccttccagccgtccgacccgatgctcaccatggtggtggactccggcctcaaggacgacctcgtgctcggcgacccgaccgccccgcgcttcgtgctctggaacggcaagctccgcccggtgccgtccaagctcaccgacctcccgttcttcgacctcatgtccatcggcggcaagatccgcgccggcttcggcgccctcggcatccgcccgtccccgccgggccgcgaggagtccgtggaggagttcgtgcgccgcaacctcggcgacgaggtgttcgagcgcctcatcgagccgttctgctccggcgtgtacgccggcgacccgtccaagctctccatgaaggccgccttcggcaaggtgtggaagctcgagcagaacggcggctccatcatcggcggcaccttcaaggccatccaggagcgcaagaacgccccgaaggccgagcgcgacccgcgcctcccgaagccgcagggccagaccgtgggctccttccgcaagggcctccgcatgctcccggaggccatctccgcccgcctcggctccaaggtgaagctctcctggaagctcctcggcatcaccaagctcgagtccggcggctacaacctcacctacgagaccccggacggcctcgtgtccgtgcagtccaagtccgtggtgatgaccgtgccgtcccacgtggcctccggcctcctccgcccgctctccgagtccgccgccaacgccctctccaagctctactacccgccggtggccgccgtgtccatctcctacccgaaggaggccatccgcaccgagtgcctcatcgacggcgaactcaagggcttcggccagctccacccgcgcacccagggcgtggagaccctcggcaccatctactcctcctccctcttcccgaaccgcgccccgccgggccgcatcctcctcctcaacatgatcggcggctccaccaacaccggcatcctctccaagtccgagggcgaactcgtggaggccgtggaccgcgacctccgcaagatgctcatcaagccgaactccaccgacccgctcaagctcggcgtgcgcgtgtggccgcaggccatcccgcagttcctcgtgggccacttcgacatcctcgacaccgccaagtcctccctcacctcctccggctacgagggcctcttcctcggcggcaactacgtggccggcgtggccctcggccgctgcgtggagggcgcctacgagaccgccatcgaggtgaacaacttcatgtcccgctacgcctacaagtaagcagagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgtagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 181 - DNA, 636 bp, Brachybacterium distachyon
atggaggtgctgagcgggagggtaggggtgaagtgcgggcggtggaacccgacggcggagcaggtgaaggtgctgacggagcttttccgggcggggctgcggacgccgagcacggagcagatccagcggatctccacccacctcggcgccttcggcaaggtggagagcaagaacgtcttctactggttccagaaccacaaggcccgcgagcgccaccaccacaagaagcgccgccgcgtcgcctcctcctcctccgacaacagcagcgccagcaacaacaacgacgaagcagcagatcacggccgcagtagtgcccgcgaggacctcctgctgcagcctcccgagagcaagcgcgaggccagaagctacaaccaccaccgccggccgatcatgacatatgtaggctatgtgagggacgaggtggagcaggaggtggtgatgtgggagcggccgacgagggaggtggagacgctcgagctgttcccgctcaaggcagcctacgacctcgaggcggcggacaggctccggtacgtgaggggcgccggcgagcagcagtgcagggagatctccttcttcgacgtcgccaacggacgggatccgccgctggagctcaggctctgcagcttcgatatctag

SEQ ID NO: 182 - DNA, 1994 bp, Zea mays
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcctaggtacgccgctcgtcctccccccccccccctctctaccttctctagatcggcgttccggtccatagttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatccagcagctatatgtggatttttttagccctgccttcatacgctatttatttgcttggtactgtttcttttgtcgatgctcaccctgttgtttggtgttacttctgcag

SEQ ID NO: 183 - DNA, 29 bp, Artificial Sequence
  FE12949 forward primer
atcgatctgtcacttgattttaattagaa

SEQ ID NO: 184 - DNA, 21 bp, Artificial Sequence
  FE12950 reverse primer
gtgagcggctttcctgtatct

SEQ ID NO: 185 - DNA, 18 bp, Artificial Sequence
  FE12951 probe
tctggctgattctctatt

SEQ ID NO: 186 - DNA, 20 bp, Artificial Sequence
  FE12703 forward primer
tgtagtccgttccagcgaca

SEQ ID NO: 187 - DNA, 21 bp, Artificial Sequence
  FE12704 reverse primer
ggtttcaggtttggggaaaga

SEQ ID NO: 188 - DNA, 17 bp, Artificial Sequence
  FE12705 probe
ccgtagatcttctcctc

SEQ ID NO: 189 - DNA, 23 bp, Artificial Sequence
  FE12958 forward primer
gctgactggtctcaccatctcat

SEQ ID NO: 190 - DNA, 29 bp, Artificial Sequence
  FE12959 reverse primer
agtatgatggccatatatttgctatctta

SEQ ID NO: 191 - DNA, 18 bp, Artificial Sequence
  FE12960 probe
ctaaagatggtggtggtg

SEQ ID NO: 192 - DNA, 20 bp, Artificial Sequence
  FE12952 forward primer
tgggacatttgggaaggaaa

SEQ ID NO: 193 - DNA, 20 bp, Artificial Sequence
  FE12953 reverse primer
gtctaagggcctgctgacga

SEQ ID NO: 194 - DNA, 18 bp, Artificial Sequence
  FE12954 probe
ctgaaggaaacctatctc

SEQ ID NO: 195 - DNA, 20 bp, Artificial Sequence
  FE12952 forward primer
tgggacatttgggaaggaaa

SEQ ID NO: 196 - DNA, 19 bp, Artificial Sequence
  FE12956 reverse primer
ctgactgcctgatgacgca

SEQ ID NO: 197 - DNA, 19 bp, Artificial Sequence
  FE12957 probe
ttggtatgtttgcctcaga